(19) United States
(12) Patent Application Publication
(10) Pub. No.: US 2023/0278997 A1
(43) Pub. Date: Sep. 7, 2023

(54) BICYCLO[1.1.1]PENTANE COMPOUNDS FOR THE TREATMENT AND PROPHYLAXIS OF HEPATITIS B VIRUS INFECTION

(71) Applicant: Hoffmann-La Roche Inc., Little Falls, NJ (US)

(72) Inventors: Jianping WANG, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/318,144

(22) Filed: May 16, 2023

Related U.S. Application Data
(63) Continuation of application No. PCT/EP2021/081756, filed on Nov. 16, 2021.

(30) Foreign Application Priority Data
Nov. 19, 2020 (WO) PCT/CN2020/130142

(51) Int. Cl. C07D 417/12; C07D 417/14; C07D 413/12; C07D 405/12; C07D 409/12

(52) U.S. Cl. CPC C07D 417/12; C07D 417/14; C07D 413/12; C07D 405/12; C07D 409/12

(57) ABSTRACT

The present invention provides novel compounds having the general formula (I):

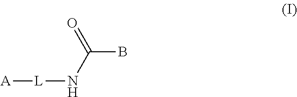

wherein A, L, and B are as described herein, compositions including the compounds and methods of using the compounds.

### CROSS-REFERENCE TO PRIOR APPLICATIONS

This application is a continuation of International Application No. PCT/EP2021/081756 having an International Filing Date of Nov. 16, 2021 and which claims benefit under 35 U.S.C. § 119 to International Application No. PCT/CN2020/130142 having an International Filing Date of Nov. 19, 2020. The entire contents of both are incorporated herein by reference.

### FIELD OF THE INVENTION

The present invention relates to organic compounds useful for therapy and/or prophylaxis of HBV infection in a mammal, and in particular to HBsAg (HBV Surface antigen) and HBeAg (HBV e antigen) inhibitors useful for treating an HBV infection. In particular, the present invention relates to bicyclo[1.1.1]pentane compounds and their corresponding derivatives that have anti-virus activity, as well as their manufacture and pharmaceutical compositions containing them.

### BACKGROUND OF THE INVENTION

Hepatitis B virus (HBV) is one of the most dangerous human pathogens. A safe and effective vaccine has been available for longer than two decades; however, WHO estimated that approximately 257 million people are chronically infected with HBV. Chronic Hepatitis B (CHB) infection predisposes its host to severe liver disease, including liver cirrhosis and hepatocellular carcinoma, if left untreated. HBV infection is ranked among the top unmet medical need worldwide. The currently approved drugs have contributed to substantial progress in CHB treatment; however, the cure rate remains less than 10%.

The control of viral infection needs an effective immune surveillance. Upon recognition of viral infection, the host innate immune system could respond within minutes to impede viral replication and limits the development of a chronic and persistent infection. The secretion of antiviral cytokines from infected hepatocytes and intra-hepatic immune cells is critically important for the clearance of viral infection. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may contribute to immune tolerant state observed in CHB patients. The persistent exposure to HBsAg and other viral antigens can lead to HIBV-specific T-cell functional impairment and depletion (Kondo et al. Journal of Immunology (1993), 150, 4659-4671; Kondo et al. Journal of Medical Virology (2004), 74, 425-433; Fisicaro et al. Gastroenterology, (2010), 138, 682-693). Moreover, HBsAg has been reported to suppress immune cell functions, including monocytes, dendritic cells (DCs) and natural killer (NK) cells (Op den Brouw et al. Immunology, (2009b), 126, 280-289; Woltman et al. PLoS One, (2011), 6, e15324; Shi et al. J Viral Hepat. (2012), 19, e26-33; Kondo et al. ISRN Gasteroenterology, (2013), Article ID 935295).

HBsAg is an important biomarker for prognosis and treatment response in CUB. However, the achievement of HBsAg loss and seroconversion is rarely achieved in CHB patients. HBsAg loss with or without anti-HBsAg seroconversion remains the ideal clinical treatment endpoints. Current therapies, such as nucleos(t)ide analogues, are effective in supressing HBV DNA, but are not effective in reducing HBsAg level. Nucleos(t)ide analogues, even with prolonged therapy, have demonstrated HBsAg clearance rates comparable to those observed naturally (Janssen et al. Lancet, (2005), 365, 123-129; Marcellin et al. N. Engl. J. Med., (2004), 351, 1206-1217; Buster et al. Hepatology, (2007), 46, 388-394). Therefore, there is an urgent need for the development of novel therapeutic agents that could efficiently reduce HBsAg (Wieland, S. F. & F. V. Chisari. J Virol, (2005), 79, 9369-9380; Kumar et al. J Virol, (2011), 85, 987-995; Woltman et al. PLoS One, (2011), 6, e15324; Op den Brouw et al. Immunology, (2009b), 126, 280-289).

### SUMMARY OF THE INVENTION

Objects of the present invention are novel compounds of formula (I), their manufacture, medicaments based on a compound in accordance with the invention and their production as well as the use of compounds of formula (I) as HBV inhibitors and for the treatment or prophylaxis of HBV infection. The compounds of formula (I) show superior anti-HBV activity. In addition, the compounds of formula (I) also show good safety and good PK profiles.

The present invention relates to a compound of formula (I),

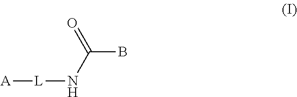

wherein
A is a 5 or 6 membered heteroaryl containing 1, 2, 3, or 4 heteroatom selected from N, O, and S; wherein A is substituted with R¹, or substituted with both R¹ and R², and wherein
   R¹ is hydrogen, halogen, C₁₋₆alkyl, haloC₁₋₆alkyl, C₃₋₇cycloalkyl, phenyl, pyridinyl, or pyrimidinyl, wherein each of said phenyl, pyridinyl, and pyrimidinyl is unsubstituted or substituted with one or two substituents independently selected from halogen, C₁₋₆alkyl, and C₁₋₆alkoxy;
   R² is hydrogen, halogen, or C₁₋₆alkyl;
L is a C₅₋₁₂cycloalkyl, wherein L is a monocyclic ring or a bicyclic ring, and wherein the bicyclic ring is a bridged, spiro or fused ring;
B is a phenyl, dioxothiolanyl, or a 5 or 6 membered heteroaryl containing 1, 2, 3, or 4 heteroatoms independently selected from N, O, and S; wherein B is substituted with R³, and wherein
   R³ is hydrogen, C₁₋₆alkylsulfonylC₃₋₇cycloalkyl-, C₁₋₆alkylsulfonylC₁₋₆alkyl-, or carboxamide;
      or a pharmaceutically acceptable salt thereof.

### DETAILED DESCRIPTION OF THE INVENTION

### Definitions

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

The nomenclature used in this application is based on IUPAC systematic nomenclature, unless indicated otherwise.

The term “chiral” denotes the ability of non-superimposability with the mirror image, while the term “achiral” refers to embodiments which are superimposable with their mirror image. Chiral molecules are optically active, i.e., they have the ability to rotate the plane of plane-polarized light. Whenever a chiral center is present in a chemical structure, it is intended that all stereoisomers associated with that chiral center are encompassed by the present invention.

The term “compound(s) of this invention” and “compound(s) of the present invention” refers to compounds of formula (I) and stereoisomers, solvates or salts thereof (e.g., pharmaceutically acceptable salts).

The term “substituent” denotes an atom or a group of atoms replacing a hydrogen atom on the parent molecule.

As used herein, the term “C₁₋₆alkyl” alone or in combination signifies a saturated, linear- or branched chain alkyl group containing 1 to 6, particularly 2 to 6 or 1 to 4 carbon atoms, for example methyl, ethyl, propyl, isopropyl, butyl, isobutyl, tert-butyl and the like. Particular “C₁₋₆alkyl” groups are methyl and ethyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy and propoxy.

The term “C₃₋₇cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” group is cyclopropyl. The term “C₅₋₁₂cycloalkyl” denotes to a saturated carbon ring containing from 5 to 12 carbon atoms, for example, bicyclo[1.1.1]pentanyl.

The term “halogen” denotes fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl.

The term “carbonyl” alone or in combination refers to the group —C(O)—.

The term “heteroaryl” denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl moieties include, but not limited to, pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl or quinoxalinyl. Heteroaryl can be further substituted by halogen, C₁₋₆alkyl, haloC₁₋₆alkyl, cyano, C₃₋₇cycloalkyl, (C₁₋₆ alkyl)₂amino or C₁₋₆alkoxy.

The term “sulfonyl” alone or in combination refers to the group —S(O)₂—.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### HBV Inhibitors

The present invention relates to (i) a novel compound of formula (I),

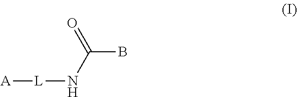

wherein
A is a 5 or 6 membered heteroaryl containing 1, 2, 3, or 4 heteroatom selected from N, O, and S; wherein A is substituted with R¹, or substituted with both R¹ and R², and wherein
   R¹ is hydrogen, halogen, C₁₋₆alkyl, haloC₁₋₆alkyl, C₃₋₇cycloalkyl, phenyl, pyridinyl, or pyrimidinyl, wherein each of said phenyl, pyridinyl, and pyrimidinyl is unsubstituted or substituted with one or two substituents independently selected from halogen, C₁₋₆ alkyl, or C₁₋₆alkoxy;
   R² is hydrogen, halogen, or C₁₋₆alkyl;
L is a C₅₋₁₂cycloalkyl, wherein L is a monocyclic ring or a bicyclic ring, and wherein the bicyclic ring is a bridged, spiro or fused ring;
B is a phenyl, dioxothiolanyl, or a 5 or 6 membered heteroaryl containing 1, 2, 3, or 4 heteroatoms independently selected from N, O, and S; wherein B is substituted with R³, and wherein
   R³ is hydrogen, C₁₋₆alkylsulfonylC₃₋₇cycloalkyl-, C₁₋₆alkylsulfonylC₁₋₆alkyl-, or carboxamide;
or a pharmaceutically acceptable salt thereof.

A further embodiment of present invention is (ii) a compound of formula (I), or a pharmaceutically acceptable salt thereof, according to (i), wherein
A is thiazolyl, thiadiazolyl, oxadizolyl, pyrazolyl, trizolyl, tetrazolyl, or pyrimidinyl; wherein A is substituted with R¹, or substituted with both R¹ and R², and wherein
   R¹ is hydrogen, halogen, C₁₋₆alkyl, haloC₁₋₆alkyl, C₃₋₇cycloalkyl, phenyl, pyridinyl, or pyrimidinyl, wherein each of said phenyl, pyridinyl, and pyrimidinyl is unsubstituted or substituted with one or two substituents independently selected from halogen, C₁₋₆alkyl or C₁₋₆alkoxy;
   R² is hydrogen, halogen, or C₁₋₆alkyl.

A further embodiment of present invention is (iii) a compound of formula (I), or a pharmaceutically acceptable salt thereof, according to (ii), wherein
A is thiazolyl, thiadiazolyl, oxadizolyl, pyrazolyl, trizolyl, tetrazolyl, or pyrimidinyl; wherein A is substituted with R¹, and wherein
   R¹ is phenyl or pyridinyl, wherein each of said phenyl and pyridinyl is substituted once or twice with halogen.

A further embodiment of present invention is (iv) a compound of formula (I), or a pharmaceutically acceptable salt thereof, according to (iii), wherein
A is 4-(4-chlorophenyl)thiazol-2-yl, 4-(3-chlorophenyl)thiazol-2-yl, 4-(3,4-dichlorophenyl)thiazol-2-yl, 4-(5-chloro-2-pyridyl)thiazol-2-yl, 3-(4-chlorophenyl)-1,2,4-thiadiazol-5-yl, 5-(4-chlorophenyl)thiazol-2-yl, 5-(3,4-dichlorophenyl)thiazol-2-yl, 2-(4-chlorophenyl)thiazol-4-yl, 2-(4-chlorophenyl)triazol-4-yl, 3-(4-chlorophenyl)-1,2,4-oxadiazol-5-yl, 3-(4-fluorophenyl)-1,2,4-oxadiazol-5-yl, 5-(4-chlorophenyl)-1,2,4-oxadiazol-3-yl, 1-(4-chlorophenyl)pyrazol-3-yl, 2-(4-chlorophenyl)tetrazol-5-yl, or 2-(4-chlorophenyl)pyrimidin-4-yl.

A further embodiment of present invention is (v) a compound of formula (I), or a pharmaceutically acceptable salt thereof, according to (ii), wherein
A is thiazolyl, thiadiazolyl, oxadizolyl, pyrazolyl, or trizolyl; wherein A is substituted with R¹, and wherein
   R¹ is phenyl or pyridinyl, wherein each of said phenyl and pyridinyl is substituted once with halogen.

A further embodiment of present invention is (vi) A compound of formula (I), or a pharmaceutically acceptable salt thereof, according to (v), wherein
A is 4-(4-chlorophenyl)thiazol-2-yl, 4-(3-chlorophenyl)thiazol-2-yl, 4-(5-chloro-2-pyridyl)thiazol-2-yl, 3-(4-chlorophenyl)-1,2,4-thiadiazol-5-yl, 5-(4-chlorophenyl)thiazol-2-yl, 2-(4-chlorophenyl)triazol-4-yl, 5-(4-chlorophenyl)-1,2,4-oxadiazol-3-yl, or 1-(4-chlorophenyl)pyrazol-3-yl.

A further embodiment of present invention is (vii) a compound of formula (I), or a pharmaceutically acceptable salt thereof, according to any one of (i) to (vi), wherein
L is

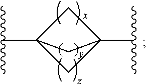

each of x, y, and z is independently an integer of 1, 2, or 3.

A further embodiment of present invention is (viii) a compound of formula (I), or a pharmaceutically acceptable salt thereof, according to (vii), wherein
L is

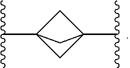 [SMILES: *C12CC(*)(C1)C2]

A further embodiment of present invention is (ix) a compound of formula (I), or a pharmaceutically acceptable salt thereof, according to any one of (i) to (viii), wherein
B is furanyl, oxazolyl, oxadizolyl, thiadiazolyl, pyrazolyl, pyridinyl, phenyl, pyridinyl, or dioxothiolanyl; wherein B is substituted with R³, and wherein
   R³ is hydrogen, C₁₋₆alkylsulfonylC₃₋₇cycloalkyl-, C₁₋₆alkylsulfonylC₁₋₆alkyl-, or carboxamide.

A further embodiment of present invention is (x) a compound of formula (I), or a pharmaceutically acceptable salt thereof, according to (ix), wherein
B is furanyl, oxazolyl, thiadiazolyl, pyrazolyl, or pyridinyl; wherein B is substituted with R³, and wherein
   R³ is C₁₋₆alkylsulfonylC₃₋₇cycloalkyl- or C₁₋₆alkylsulfonylC₁₋₆alkyl-.

A further embodiment of present invention is (xi) a compound of formula (I), or a pharmaceutically acceptable salt thereof, according to (x), wherein
B is 5-(1-methylsulfonylcyclopropyl)furan-2-yl, 3-(1-methylsulfonylcyclopropyl)-1,2,4-thiadiazole-5-yl, 2-(1-methylsulfonylcyclopropyl)oxazole-5-yl, 3-(methylsulfonylmethyl)-1,2,4-thiadiazole-5-yl, 3-(1-methyl-1-methylsulfonyl-ethyl)phenyl, 3-(1-methylsulfonylcyclopropyl)pyrazol-1-yl, or 2-(1-methylsulfonylcyclopropyl)oxazol-5-yl.

A further embodiment of present invention is (xii) a compound of formula (I), or a pharmaceutically acceptable salt thereof, according to (xi), wherein
B is furanyl or thiadiazolyl; wherein B is substituted with R³, and wherein R³ is C₁₋₆alkylsulfonylC₃₋₇cycloalkyl-.

A further embodiment of present invention is (xiii) a compound of formula (I), or a pharmaceutically acceptable salt thereof, according to (xii), wherein
B is 5-(1-methylsulfonylcyclopropyl)furan-2-yl or 3-(1-methylsulfonylcyclopropyl)-1,2,4-thiadiazole-5-yl.

A further embodiment of present invention is (xiv) a compound of formula (I), or a pharmaceutically acceptable salt thereof, according to any one of (i) to (xiii), wherein
A is thiazolyl, thiadiazolyl, oxadizolyl, pyrazolyl, or trizolyl; wherein A is substituted with R¹, and wherein
   R¹ is phenyl or pyridinyl, wherein each of said phenyl and pyridinyl is substituted once with halogen;
L is

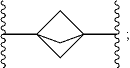 [SMILES: *C12CC(*)(C1)C2]

and
B is furanyl or thiadiazolyl; wherein B is substituted with R³, and wherein
   R³ is C₁₋₆alkylsulfonylC₃₋₇cycloalkyl-.

A further embodiment of present invention is (xv) a compound of formula (I), or a pharmaceutically acceptable salt thereof, according to (xiv), wherein
A is 4-(4-chlorophenyl)thiazol-2-yl, 4-(3-chlorophenyl)thiazol-2-yl, 4-(5-chloro-2-pyridyl)thiazol-2-yl, 3-(4-chlorophenyl)-1,2,4-thiadiazol-5-yl, 5-(4-chlorophenyl)thiazol-2-yl, 2-(4-chlorophenyl)triazol-4-yl, 5-(4-chlorophenyl)-1,2,4-oxadiazol-3-yl, or 1-(4-chlorophenyl)pyrazol-3-yl;
L is

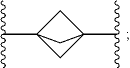 [SMILES: *C12CC(*)(C1)C2]

and
B is 5-(1-methylsulfonylcyclopropyl)furan-2-yl or 3-(1-methylsulfonylcyclopropyl)-1,2,4-thiadiazole-5-yl.

A further embodiment of present invention is (xvi) a compound selected from:
N-[3-[4-(4-chlorophenyl)thiazol-2-yl]-1-bicyclo[1.1.1]pentanyl]-5-(1-methylsulfonylcyclopropyl)furan-2-carboxamide;
N-[3-[4-(3-chlorophenyl)thiazol-2-yl]-1-bicyclo[1.1.1]pentanyl]-5-(1-methylsulfonylcyclopropyl)furan-2-carboxamide;
N-[3-[4-(2-chlorophenyl)thiazol-2-yl]-1-bicyclo[1.1.1]pentanyl]-5-(1-methylsulfonylcyclopropyl)furan-2-carboxamide;
N-[3-[4-(3-chlorophenyl)thiazol-2-yl]-1-bicyclo[1.1.1]pentanyl]-3-(1-methylsulfonylcyclopropyl)-1,2,4-thiadiazole-5-carboxamide;
N-[3-[4-(4-chlorophenyl)thiazol-2-yl]-1-bicyclo[1.1.1]pentanyl]-2-(methylsulfonylmethyl)oxazole-5-carboxamide;
N-[3-[4-(4-chlorophenyl)thiazol-2-yl]-1-bicyclo[1.1.1]pentanyl]-2-(1-methylsulfonylcyclopropyl)oxazole-5-carboxamide;
N-[3-[4-(4-chlorophenyl)thiazol-2-yl]-1-bicyclo[1.1.1]pentanyl]-5-(1-methylsulfonylcyclopropyl)-1,3,4-oxadiazole-2-carboxamide;
N-[3-[4-(3,4-dichlorophenyl)thiazol-2-yl]-1-bicyclo[1.1.1]pentanyl]-5-(1-methylsulfonylcyclopropyl)furan-2-carboxamide;
N-[3-[4-(6-chloro-3-pyridyl)thiazol-2-yl]-1-bicyclo[1.1.1]pentanyl]-5-(1-methylsulfonylcyclopropyl)furan-2-carboxamide;
N-[3-[4-(5-chloro-2-pyridyl)thiazol-2-yl]-1-bicyclo[1.1.1]pentanyl]-5-(1-methylsulfonylcyclopropyl)furan-2-carboxamide;
5-(1-methylsulfonylcyclopropyl)-N-[3-[4-(2-pyridyl)thiazol-2-yl]-1-bicyclo[1.1.1]pentanyl]furan-2-carboxamide;
5-(1-methylsulfonylcyclopropyl)-N-[3-(4-pyrimidin-5-ylthiazol-2-yl)-1-bicyclo[1.1.1]pentanyl]furan-2-carboxamide;
5-(1-methylsulfonylcyclopropyl)-N-[3-(4-phenylthiazol-2-yl)-1-bicyclo[1.1.1]pentanyl]furan-2-carboxamide;
N-[3-(4-cyclopropylthiazol-2-yl)-1-bicyclo[1.1.1]pentanyl]-5-(1-methylsulfonylcyclopropyl)furan-2-carboxamide;
N-[3-(4-bromothiazol-2-yl)-1-bicyclo[1.1.1]pentanyl]-5-(1-methylsulfonylcyclopropyl)furan-2-carboxamide;
5-(1-methylsulfonylcyclopropyl)-N-[3-[4-(trifluoromethyl)thiazol-2-yl]-1-bicyclo[1.1.1]pentanyl]furan-2-carboxamide;
5-(1-methylsulfonylcyclopropyl)-N-[3-(3-phenyl-1,2,4-thiadiazol-5-yl)-1-bicyclo[1.1.1]pentanyl]furan-2-carboxamide;
N-[3-[3-(4-chlorophenyl)-1,2,4-thiadiazol-5-yl]-1-bicyclo[1.1.1]pentanyl]-5-(1-methylsulfonylcyclopropyl)furan-2-carboxamide;
N-[3-[5-(4-chlorophenyl)thiazol-2-yl]-1-bicyclo[1.1.1]pentanyl]-5-(1-methylsulfonylcyclopropyl)furan-2-carboxamide;
N-[3-[5-(4-chlorophenyl)thiazol-2-yl]-1-bicyclo[1.1.1]pentanyl]-5-(1-methylsulfonylcyclopropyl)-1,3,4-oxadiazole-2-carboxamide;
N-[3-[5-(3,4-dichlorophenyl)thiazol-2-yl]-1-bicyclo[1.1.1]pentanyl]-5-(1-methylsulfonylcyclopropyl)furan-2-carboxamide;
5-(1-methylsulfonylcyclopropyl)-N-[3-(5-phenylthiazol-2-yl)-1-bicyclo[1.1.1]pentanyl]furan-2-carboxamide;
N-[3-(5-cyclopropylthiazol-2-yl)-1-bicyclo[1.1.1]pentanyl]-5-(1-methylsulfonylcyclopropyl)furan-2-carboxamide;
N-[3-(5-bromothiazol-2-yl)-1-bicyclo[1.1.1]pentanyl]-5-(1-methylsulfonylcyclopropyl)furan-2-carboxamide;
5-(1-methylsulfonylcyclopropyl)-N-[3-(5-phenyl-1,2,4-thiadiazol-3-yl)-1-bicyclo[1.1.1]pentanyl]furan-2-carboxamide;
5-(1-methylsulfonylcyclopropyl)-N-[3-(2-phenylthiazol-4-yl)-1-bicyclo[1.1.1]pentanyl]furan-2-carboxamide;
N-[3-[2-(4-chlorophenyl)thiazol-4-yl]-1-bicyclo[1.1.1]pentanyl]-5-(1-methylsulfonylcyclopropyl)furan-2-carboxamide;
N-[3-[5-(4-chlorophenyl)-1,3,4-oxadiazol-2-yl]-1-bicyclo[1.1.1]pentanyl]-5-(1-methylsulfonylcyclopropyl)furan-2-carboxamide;
N-[3-[1-(4-chlorophenyl)triazol-4-yl]-1-bicyclo[1.1.1]pentanyl]-5-(1-methylsulfonylcyclopropyl)furan-2-carboxamide;
N-[3-[2-(4-chlorophenyl)triazol-4-yl]-1-bicyclo[1.1.1]pentanyl]-5-(1-methylsulfonylcyclopropyl)furan-2-carboxamide;
N-[3-[2-(4-chlorophenyl)triazol-4-yl]-1-bicyclo[1.1.1]pentanyl]-1,1-dioxo-thiolane-3-carboxamide;
N-[3-[3-(4-chlorophenyl)-1,2,4-oxadiazol-5-yl]-1-bicyclo[1.1.1]pentanyl]-5-(1-methylsulfonylcyclopropyl)furan-2-carboxamide;
N-[3-[3-(4-chlorophenyl)-1,2,4-oxadiazol-5-yl]-1-bicyclo[1.1.1]pentanyl]-3-(methylsulfonylmethyl)-1,2,4-thiadiazole-5-carboxamide;
N-[3-[3-(4-chlorophenyl)-1,2,4-oxadiazol-5-yl]-1-bicyclo[1.1.1]pentanyl]-3-(1-methylsulfonylcyclopropyl)-1,2,4-thiadiazole-5-carboxamide;
N-[3-[3-(4-fluorophenyl)-1,2,4-oxadiazol-5-yl]-1-bicyclo[1.1.1]pentanyl]-3-(1-methylsulfonylcyclopropyl)-1,2,4-thiadiazole-5-carboxamide;
N4-[3-[3-(4-chlorophenyl)-1,2,4-oxadiazol-5-yl]-1-bicyclo[1.1.1]pentanyl]pyridine-2,4-dicarboxamide;
N-[3-[3-(4-chlorophenyl)-1,2,4-oxadiazol-5-yl]-1-bicyclo[1.1.1]pentanyl]-3-(1-methyl-1-methylsulfonyl-ethyl)benzamide;
N-[3-[3-(4-chlorophenyl)-1,2,4-oxadiazol-5-yl]-1-bicyclo[1.1.1]pentanyl]-6-(1-methyl-1-methylsulfonyl-propyl)pyridine-2-carboxamide;
N-[3-[3-(4-chlorophenyl)-1,2,4-oxadiazol-5-yl]-1-bicyclo[1.1.1]pentanyl]-2-(1-methyl-1-methylsulfonyl-propyl)pyridine-4-carboxamide;
N-[3-[5-(4-chlorophenyl)-1,2,4-oxadiazol-3-yl]-1-bicyclo[1.1.1]pentanyl]-5-(1-methylsulfonylcyclopropyl)furan-2-carboxamide;
N-[3-[5-(4-chlorophenyl)-1,2,4-oxadiazol-3-yl]-1-bicyclo[1.1.1]pentanyl]-3-(1-methylsulfonylcyclopropyl)-1,2,4-thiadiazole-5-carboxamide;
N-[3-[1-(4-chlorophenyl)pyrazol-3-yl]-1-bicyclo[1.1.1]pentanyl]-5-(1-methylsulfonylcyclopropyl)furan-2-carboxamide;
N-[3-[1-(4-chlorophenyl)pyrazol-3-yl]-1-bicyclo[1.1.1]pentanyl]-3-(1-methylsulfonylcyclopropyl)-1,2,4-thiadiazole-5-carboxamide;
N-[3-[2-(4-chlorophenyl)tetrazol-5-yl]-1-bicyclo[1.1.1]pentanyl]-5-(1-methylsulfonylcyclopropyl)furan-2-carboxamide;
N-[3-[4-(4-chlorophenyl)pyrimidin-2-yl]-1-bicyclo[1.1.1]pentanyl]-5-(1-methylsulfonylcyclopropyl)furan-2-carboxamide;
N-[3-[4-(4-chlorophenyl)pyrimidin-2-yl]-1-bicyclo[1.1.1]pentanyl]-3-(1-methylsulfonylcyclopropyl)-1,2,4-thiadiazole-5-carboxamide;
N-[3-[2-(4-chlorophenyl)pyrimidin-4-yl]-1-bicyclo[1.1.1]pentanyl]-5-(1-methylsulfonylcyclopropyl)furan-2-carboxamide;
N-[3-[2-(4-chlorophenyl)pyrimidin-4-yl]-1-bicyclo[1.1.1]pentanyl]-3-(1-methylsulfonylcyclopropyl)-1,2,4-thiadiazole-5-carboxamide;
N-[3-[5-bromo-2-(4-chlorophenyl)triazol-4-yl]-1-bicyclo[1.1.1]pentanyl]-5-(1-methylsulfonylcyclopropyl)furan-2-carboxamide;
N-[3-[2-(4-chlorophenyl)-5-methyl-triazol-4-yl]-1-bicyclo[1.1.1]pentanyl]-5-(1-methylsulfonylcyclopropyl)furan-2-carboxamide;
3-(1-methylsulfonylcyclopropyl)-N-[3-[2-(p-tolyl)triazol-4-yl]-1-bicyclo[1.1.1]pentanyl]-1,2,4-thiadiazole-5-carboxamide;
N-[3-[2-(4-methoxyphenyl)triazol-4-yl]-1-bicyclo[1.1.1]pentanyl]-3-(1-methylsulfonylcyclopropyl)-1,2,4-thiadiazole-5-carboxamide;
N-[3-[4-(4-chlorophenyl)thiazol-2-yl]-1-bicyclo[1.1.1]pentanyl]-3-(1-methylsulfonylcyclopropyl)pyrazole-1-carboxamide;
N-[3-[5-(4-chlorophenyl)thiazol-2-yl]-1-bicyclo[1.1.1]pentanyl]-3-(1-methylsulfonylcyclopropyl)pyrazole-1-carboxamide;
N-[3-[5-(4-chlorophenyl)thiazol-2-yl]-1-bicyclo[1.1.1]pentanyl]-2-(1-methylsulfonylcyclopropyl)oxazole-5-carboxamide;
N-[3-[5-(6-chloro-3-pyridyl)thiazol-2-yl]-1-bicyclo[1.1.1]pentanyl]-5-(1-methylsulfonylcyclopropyl)furan-2-carboxamide;
N-[3-[5-(5-chloro-2-pyridyl)thiazol-2-yl]-1-bicyclo[1.1.1]pentanyl]-5-(1-methylsulfonylcyclopropyl)furan-2-carboxamide;
N-[3-[5-(5-chloropyrimidin-2-yl)thiazol-2-yl]-1-bicyclo[1.1.1]pentanyl]-5-(1-methylsulfonylcyclopropyl)furan-2-carboxamide;
N-[3-[5-(5-chloropyrazin-2-yl)thiazol-2-yl]-1-bicyclo[1.1.1]pentanyl]-5-(1-methylsulfonylcyclopropyl)furan-2-carboxamide;
N-[3-[2-(4-chlorophenyl)triazol-4-yl]-1-bicyclo[1.1.1]pentanyl]-3-(1-methylsulfonylcyclopropyl)-1,2,4-thiadiazole-5-carboxamide;
   or a pharmaceutically acceptable salt thereof.

### Pharmaceutical Compositions and Administration

The invention also relates to a compound of formula (I) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduction of HBsAg and HBeAg in HBV patients. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a pharmaceutically acceptable salt thereof.

In a further embodiment includes a pharmaceutical composition comprising a compound of formula (I), or a pharmaceutically acceptable salt thereof, together with a pharmaceutically acceptable carrier or excipient.

Another embodiment includes a pharmaceutical composition comprising a compound of formula (I), or a pharmaceutically acceptable salt thereof for use in the treatment of HBV infection.

The following embodiments illustrate typical compositions of the present invention, but serve merely as representative thereof.

Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention have anti-HBV activity. Accordingly, the compounds of the invention are useful for the treatment or prophylaxis of HBV infection.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for use in the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of formula (I), or a pharmaceutically acceptable salt thereof.

The invention relates in particular to a compound of formula (I) for use in the treatment or prophylaxis of HBV infection.

### Synthesis

The compounds of the present invention can be prepared by any conventional means. Suitable processes for synthesizing these compounds as well as their starting materials are provided in the schemes below and in the examples. All substituents, in particular, A, L, and B are as defined above unless otherwise indicated. Furthermore, and unless explicitly otherwise stated, all reactions, reaction conditions, abbreviations and symbols have the meanings well known to a person of ordinary skill in organic chemistry.

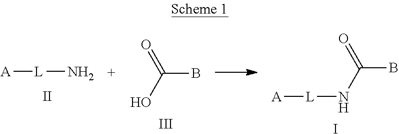

A compound of formula (I) can be prepared according to Scheme 1. Coupling of amine II and acid III with a suitable coupling reagent, such as O-(7-azabenzotriazol-1-yl)-N,N,N′,N′-tetramethyluronium hexafluorophosphate, N-ethyl-N′-(3-dimethylaminopropyl)carbodiimide hydrochloride, 1-ethyl-3-(3-dimethylaminopropyl)carbodiimide and a suitable base, such as triethylamine, N,N-diisopropylethylamine, 1,8-diazabicyclo[5.4.0]undec-7-ene in a suitable solvent, such as dichloromethane, N,N-dimethylformamide, dimethyl sulfoxide or 1-methyl-pyrrolidin-2-one to give the compound of formula (I).

A compound of formula (I) when manufactured according to the above process is also an object of the invention.

### EXAMPLES

The invention will be more fully understood by reference to the following examples. They should not, however, be construed as limiting the scope of the invention.

### Abbreviations

Abbreviations used herein are as follows:
ACN acetonitrile
BPO benzoyl peroxide
CDCl₃ deuterated chloroform
CD₃OD deuterated methanol
DIEA N,N-diisopropylethylamine
DMF dimethylformamide
DMSO-d₆ deuterated dimethylsulfoxide
EtOAc ethyl acetate
HATU O-(7-aza-1H-benzotriazole-1-yl)-N,N,N′,N′-tetramethyluronium hexafluorophosphate
HPLC high performance liquid chromatography
h hour
IC₅₀ the half maximal inhibitory concentration
LC-MS liquid chromatography-mass spectrometry
LDA lithium diisopropylamide
M molarity
MHz megahertz
mL milliliter
mmol millimole
MS (ESI) mass spectroscopy (electron spray ionization)
NA not available
NMR nuclear magnetic resonance spectroscopy
obsd. observed
PE petroleum ether
SFC supercritical fluid chromatography
TEA triethylamine
TFA trifluoroacetic acid
THF tetrahydrofuran
TI therapeutic index
T₃P 2,4,6-tripropyl-1,3,5,2,4,6-trioxatriphosphinane 2,4,6-trioxide
δ chemical shift

### GENERAL EXPERIMENTAL CONDITIONS

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Alternatively, intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC-MS spectra were obtained using an Acquity Ultra Performance LC—3100 Mass Detector or Acquity Ultra Performance LC—SQ Detector. Standard LC-MS conditions were as follows (running time 3 minutes):

Acidic condition: A: 0.1% formic acid in H₂O; B: 0.1% formic acid in acetonitrile;

Basic condition: A: 0.05% NH₃·H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (M+H)⁺.

The microwave assisted reactions were carried out in a Biotage Initiator Sixty or CEM Discover.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

### Intermediate Int-1

### 3-[4-(4-chlorophenyl)thiazol-2-yl]bicyclo[1.1.1]pentan-1-amine

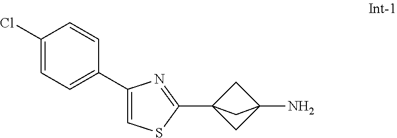 [SMILES: NC12CC(c3nc(-c4ccc(Cl)cc4)cs3)(C1)C2]

The title compound was prepared according to the following scheme:

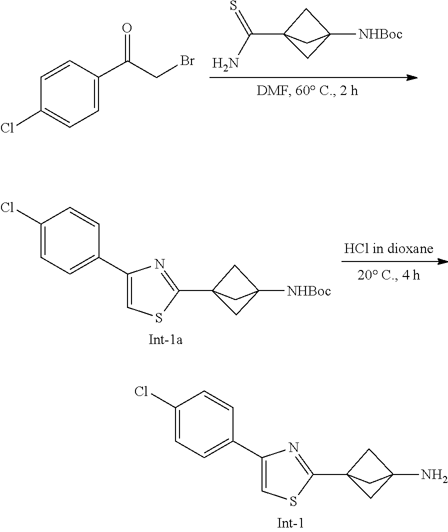 [SMILES: O=C(CBr)c1ccc(Cl)cc1 ; CC(C)(C)OC(=O)NC12CC(C(N)=S)(C1)C2 ; CC(C)(C)OC(=O)NC12CC(c3nc(-c4ccc(Cl)cc4)cs3)(C1)C2 ; NC12CC(c3nc(-c4ccc(Cl)cc4)cs3)(C1)C2]

### Step 1: Preparation tert-butyl N-[3-[4-(4-chlorophenyl)thiazol-2-yl]-1-bicyclo[1.1.1]pentanyl]carbamate (Int-1a)

To a solution of tert-butyl N-(3-carbamothioyl-1-bicyclo[1.1.1]pentanyl)carbamate (207.58 mg, 0.86 mmol) in DMF (10 mL) was added 2-bromo-1-(4-chlorophenyl)ethanone (200 mg, 0.86 mmol). The reaction was stirred at 60° C. for 2 h. After being cooled to room temperature, the resulting solution was diluted with water (30 mL) and extracted with ethyl acetate (2×20 mL). The combined organic layer was dried over Na₂SO₄ and concentrated under reduced pressure. The residue was purified by flash column (eluting with EtOAc/PE=20/80) to give tert-butyl N-[3-[4-(4-chlorophenyl)thiazol-2-yl]-1-bicyclo[1.1.1]pentanyl]carbamate (Int-1a, 300 mg, 92.93%) as a yellow solid. MS obsd. (ESI⁺) [(M+H)⁺]: 377.1.

### Step 2: Preparation of 3-[4-(4-chlorophenyl)thiazol-2-yl]bicyclo[1.1.1]pentan-1-amine (Int-1)

The solution of tert-butyl N-[3-[4-(4-chlorophenyl)thiazol-2-yl]-1-bicyclo[1.1.1]pentanyl]carbamate (Int-1a, 300 mg, 0.80 mmol) in a solution of HCl in dioxane (4 M, 5.0 mL) was stirred at 20° C. for 4 h. The mixture was concentrated under reduced pressure to give 3-[4-(4-chlorophenyl)thiazol-2-yl]bicyclo[1.1.1]pentan-1-amine (Int-1, 200 mg, HCl salt) as a white solid, which was used in the next step without further purification. MS obsd. (ESI⁺) [(M+H)⁺]: 277.1.

The following Int-2 to Int-8 were prepared in analogy to the procedure described for the preparation of Int-1, replacing 2-bromo-1-(4-chlorophenyl)ethanone with the corresponding “Ethanone” as indicated in Table 1.

TABLE 1
Compounds synthesis and characterization

| Intermediate | Compound Name and Structure | Ethanone | MS (ESI⁺) [(M + H)⁺] |
|---|---|---|---|
| Int-2 | 3-[4-(3-chlorophenyl)thiazol-2- | 2-bromo-1-(3- | 277.0 |
| | yl]bicyclo[1.1.1]pentan-1-amine | chlorophenyl)ethanone | |
| | 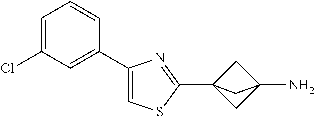 [SMILES: NC12CC(c3nc(-c4cccc(Cl)c4)cs3)(C1)C2] | | |
| Int-3 | 3-[4-(2-chlorophenyl)thiazol-2- | 2-bromo-1-(2- | 277.1 |
| | yl]bicyclo[1.1.1]pentan-1-amine | chlorophenyl)ethanone | |
| | 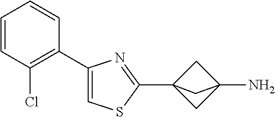 [SMILES: NC12CC(c3nc(-c4ccccc4Cl)cs3)(C1)C2] | | |
| Int-4 | 3-[4-(3,4- | 2-bromo-1-(3,4- | 311.0 |
| | dichlorophenyl)thiazol-2- | dichlorophenyl)ethanone | |
| | yl]bicyclo[1.1.1]pentan-1-amine | | |
| | 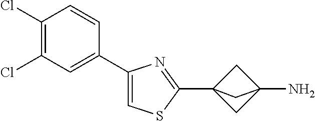 [SMILES: NC12CC(c3nc(-c4ccc(Cl)c(Cl)c4)cs3)(C1)C2] | | |
| Int-5 | 3-[4-(6-chloro-3-pyridyl)thiazol- | 2-bromo-1-(6-chloro-3- | 278.1 |
| | 2-yl]bicyclo[1.1.1]pentan-1- | pyridyl)ethanone | |
| | amine | | |
| | 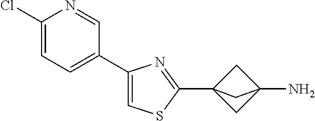 [SMILES: NC12CC(c3nc(-c4ccc(Cl)nc4)cs3)(C1)C2] | | |
| Int-6 | 3-[4-(5-chloro-2-pyridyl)thiazol- | 2-bromo-1-(5-chloro-2- | 278.2 |
| | 2-yl]bicyclo[1.1.1]pentan-1- | pyridyl)ethanone | |
| | amine | | |
| | 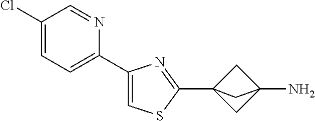 [SMILES: NC12CC(c3nc(-c4ccc(Cl)cn4)cs3)(C1)C2] | | |
| Int-7 | 3-[4-(2-pyridyl)thiazol-2- | 2-bromo-1-(2- | 244.1 |
| | yl]bicyclo[1.1.1]pentan-1-amine | pyridyl)ethanone | |
| | 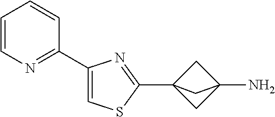 [SMILES: NC12CC(c3nc(-c4ccccn4)cs3)(C1)C2] | | |
| Int-8 | 3-(4-pyrimidin-5-ylthiazol-2- | 2-bromo-1-pyrimidin-5- | 245.1 |
| | yl)bicyclo[1.1.1]pentan-1-amine | yl-ethanone | |
| | 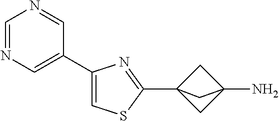 [SMILES: NC12CC(c3nc(-c4cncnc4)cs3)(C1)C2] | | |

### Intermediate Int-9

### 3-(4-phenylthiazol-2-yl)bicyclo[1.1.1]pentan-1-amine

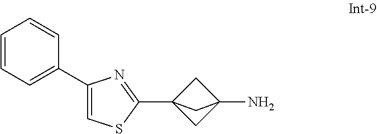 [SMILES: NC12CC(c3nc(-c4ccccc4)cs3)(C1)C2]

The title compound was prepared according to the following scheme:

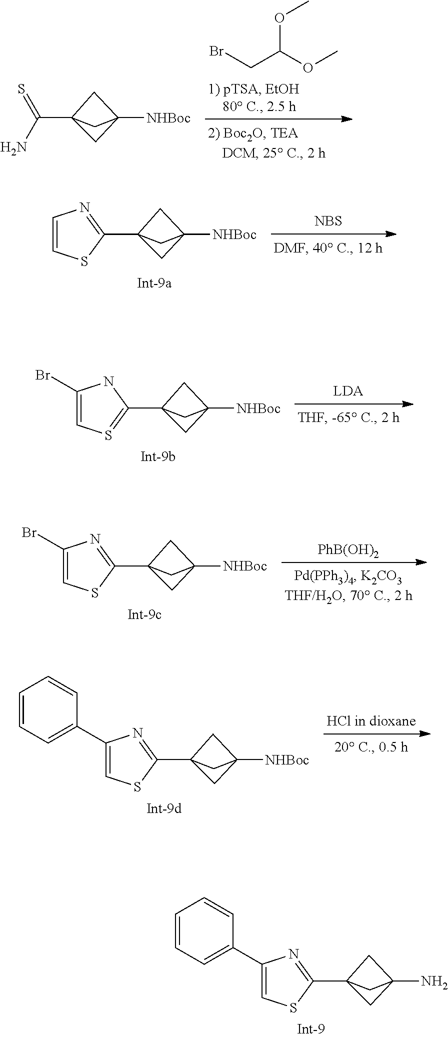

### Step 1: Preparation of tert-butyl N-(3-thiazol-2-yl-1-bicyclo[1.1.1]pentanyl)carbamate (Int-9a)

To a solution of tert-butyl N-(1-carbamothioyl-3-bicyclo[1.1.1]pentanyl)carbamate (3.0 g, 12.38 mmol) in ethanol (30 mL) was added p-toluenesulfonic acid (7.46 g, 43.33 mmol) and bromoacetaldehyde dimethyl acetal (2.93 mL, 24.76 mmol) at 25° C. The mixture was stirred at 80 10° C. for 2.5 h and then concentrated under reduced pressure. The residue was dissolved in DCM (30 mL), to which di-tert-butyl dicarbonate (3.5 g, 16.09 mmol) and TEA (2.5 g, 24.76 mmol) were added. After being stirred at 25° C. for 2 h, the resulting solution was diluted with DCM (200 mL) and washed with brine (100 mL). The organic layer was dried over anhydrous sodium sulfate and concentrated under reduced pressure. The residue was purified by flash column (eluting with EtOAc/PE=20/80) to give tert-butyl N-(1-thiazol-2-yl-3-bicyclo[1.1.1]pentanyl)carbamate (Int-9a, 887 mg, 26.9%) as a yellow solid. MS obsd. (ESI⁺) [(M+H)⁺]: 267.0.

### Step 2: Preparation of tert-butyl N-[3-(5-bromothiazol-2-yl)-1-bicyclo[1.1.1]pentanyl]carbamate (Int-9b)

To a solution of tert-butyl N-(1-thiazol-2-yl-3-bicyclo[1.1.1]pentanyl)carbamate (Int-9a, 1.0 g, 3.75 mmol) in DMF (10 mL) was added NBS (0.8 g, 4.51 mmol) at 25° C. The mixture was stirred at 40° C. for 2 h under nitrogen atmosphere. The resulting solution was concentrated under reduced pressure. The residue was purified by flash column (eluting with EtOAc/PE=30/70) to give tert-butyl N-[3-(5-bromothiazol-2-yl)-1-bicyclo[1.1.1]pentanyl]carbamate (Int-9b, 856 mg, 66.04%) as a white solid. MS obsd. (ESI⁺) [(M+H)⁺]: 345.0.

### Step 3: Preparation of tert-butyl N-[3-(4-bromothiazol-2-yl)-1-bicyclo[1.1.1]pentanyl]carbamate (Int-9c)

To a mixture of tert-butyl N-[3-(5-bromothiazol-2-yl)-1-bicyclo[1.1.1]pentanyl]carbamate (Int-9b, 30 mg, 0.09 mmol) in THF (0.5 mL) was added LDA in THF (0.1 mL, 0.20 mmol) dropwise at −65° C. under N₂, then the mixture was stirred at −65° C. under N₂ for further 2 h. The solution was poured into water (10 mL). The mixture was extracted with EtOAc (10×2 mL). The organic layer was dried over Na₂SO₄, filtered and concentrated to give tert-butyl N-[3-(4-bromothiazol-2-yl)-1-bicyclo[1.1.1]pentanyl]carbamate (Int-9c, 20 mg, 66.67%) as a yellow solid. MS obsd. (ESI⁺) [M+H]+): 347.1. ¹H NMR (400 MHz, DMSO-d₆) δ ppm: 7.12 (s, 1H), 2.46 (s, 6H), 1.47 (s, 1H).

### Step 4: Preparation of tert-butyl N-[3-(4-phenylthiazol-2-yl)-1-bicyclo[1.1.1]pentanyl]carbamate (Int-9d)

A mixture of tert-butyl N-[3-(4-bromothiazol-2-yl)-1-bicyclo[1.1.1]pentanyl]carbamate (Int-9c, 100 mg, 0.29 mmol), phenylboronic acid (50 mg, 0.41 mmol), tetrakis(triphenylphosphine)palladium(0) (30 mg, 0.03 mmol), potassium carbonate (100 mg, 0.72 mmol) in THF (2 mL) and water (0.2 mL) was heated with stirring at 70° C. under N₂ for 2 h. The mixture was concentrated to give the crude product, which was purified by flash-chromatography (eluting with EtOAc in PE from 5% to 15%) to give tert-butyl N-[3-(4-phenylthiazol-2-yl)-1-bicyclo[1.1.1]pentanyl]carbamate (Int-9d, 80 mg, 80.65%) as a white solid. MS obsd. (ESI⁺) [M+H]⁺): 343.2.

### Step 5: Preparation of 3-(4-phenylthiazol-2-yl)bicyclo[1.1.1]pentan-1-amine (Int-9)

To a mixture of tert-butyl N-[3-(4-phenylthiazol-2-yl)-1-bicyclo[1.1.1]pentanyl]carbamate (Int-9d, 80.0 mg, 0.23 mmol) in EtOAc (5 mL) was added a solution of HCl in dioxane (2.0 mL, 8 mmol). After being stirred at 15° C. for 1 h, the mixture was concentrated to give 3-(4-phenylthiazol-2-yl)bicyclo[1.1.1]pentan-1-amine (Int-9, 65.13 mg, HCl salt) as a white solid, which was used in the next step without further purification. MS obsd. (ESI⁺) [M+H]⁺: 243.0.

### Intermediate Int-10

### 3-(4-cyclopropylthiazol-2-yl)bicyclo[1.1.1]pentan-1-amine

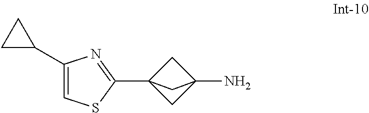 [SMILES: NC12CC(c3nc(C4CC4)cs3)(C1)C2]

The title compound was prepared in analogy to the procedure described for the preparation of 3-(4-phenylthiazol-2-yl)bicyclo[1.1.1]pentan-1-amine (Int-9), by using cyclopropylboronic acid instead of phenylboronic acid. MS obsd. (ESI⁺) [(M+H)⁺]: 207.1.

### Intermediate Int-11

### 3-(4-bromothiazol-2-yl)bicyclo[11.1.1]pentan-1-amine

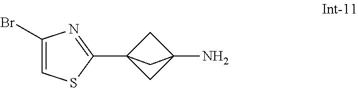 [SMILES: NC12CC(c3nc(Br)cs3)(C1)C2]

To a solution of tert-butyl N-[3-(4-bromothiazol-2-yl)-1-bicyclo[1.1.1]pentanyl]carbamate (Int-9c, 75.0 mg, 0.22 mmol) in EtOAc (3.05 mL) was added a solution of HCl in dioxane (1.02 mL, 4.06 mmol), then the solution was stirred at 20° C. for 2 h. The reaction mixture was concentrated to give crude 3-(4-bromothiazol-2-yl)bicyclo[1.1.1]pentan-1-amine (Int-11, 60 mg, HCl salt) as a yellow solid, which was used in the next step without further purification. MS obsd. (ESI⁺) [M+H⁺]: 247.3.

### Intermediate Int-12

### 3-[4-(trifluoromethyl)thiazol-2-yl]bicyclo[1.1.1]pentan-1-amine

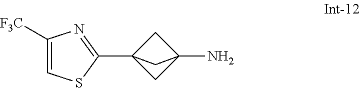 [SMILES: NC12CC(c3nc(C(F)(F)F)cs3)(C1)C2]

The title compound was prepared according to the following scheme:

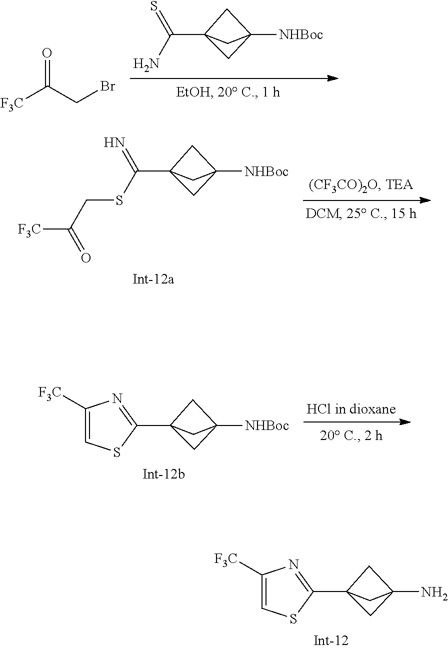 [SMILES: O=C(CBr)C(F)(F)F ; CC(C)(C)OC(=O)NC12CC(C(N)=S)(C1)C2 ; CC(C)(C)OC(=O)NC12CC(C(=N)SCC(=O)C(F)(F)F)(C1)C2 ; CC(C)(C)OC(=O)NC12CC(c3nc(C(F)(F)F)cs3)(C1)C2 ; NC12CC(c3nc(C(F)(F)F)cs3)(C1)C2]

### Step 1: Preparation of (3,3,3-trifluoro-2-oxo-propyl) 3-(tert-butoxycarbonylamino)bicyclo[1.1.1]pentane-1-carboximidothioate (Int-12a)

To a solution of tert-butyl N-(3-carbamothioyl-1-bicyclo[1.1.1]pentanyl)carbamate (130.0 mg, 0.54 mmol) in ethanol (6.5 mL) was added 3-bromo-1,1,1-trifluoro-propan-2-one (0.09 mL, 0.68 mmol). After being stirred at 20° C. for 1 h, the reaction mixture was concentrated and the residue was purified by silica gel chromatography (eluting with EtOAc in petroleum ether from 5% to 30%) to give (3,3,3-trifluoro-2-oxo-propyl) 3-(tert-butoxycarbonylamino)bicyclo[1.1.1]pentane-1-carboximidothioate (Int-12a, 140 mg, 74.06%) as a white solid. MS obsd. (ESI⁺) [(M+H)⁺]: 353.1.

### Step 2: Preparation of tert-butyl N-[3-[4-(trifluoromethyl)thiazol-2-yl]-1-bicyclo[1.1.1]pentanyl]carbamate (Int-12b)

To a solution of (3,3,3-trifluoro-2-oxo-propyl) 3-(tert-butoxycarbonylamino)bicyclo[1.1.1]pentane-1-carboximidothioate (Int-12a, 140.0 mg, 0.40 mmol) and triethylamine (100.0 mg, 0.99 mmol) in DCM (10 mL) was added trifluoroacetic anhydride (100.0 mg, 0.48 mmol) at 0° C. After being stirred at 20° C. for 2 h, the reaction was concentrated and the residue was purified by silica gel chromatography (eluting with EtOAc in petroleum ether from 5% to 25%) to give tert-butyl N-[3-[4-(trifluoromethyl)thiazol-2-yl]-1-bicyclo[1.1.1]pentanyl]carbamate (Int-12b, 60 mg, 45.17%) as colorless gum. MS obsd. (ESI⁺) [(M+H)⁺]: 335.5.

### Step 3: Preparation of 3-[4-(trifluoromethyl)thiazol-2-yl]bicyclo[1.1.1]pentan-1-amine (Int-12)

To a solution of tert-butyl N-[3-[4-(trifluoromethyl)thiazol-2-yl]-1-bicyclo[1.1.1]pentanyl]carbamate (Int-12b, 60.0 mg, 0.18 mmol) in EtOAc (3 mL) was added HCl in dioxane (1.0 mL, 4 mmol). After being stirred at 20° C. for 2 h, the reaction mixture was concentrated to give 3-[4-(trifluoromethyl)thiazol-2-yl]bicyclo[1.1.1]pentan-1-amine (Int-12, 50 mg, HCl salt) as a yellow solid, which was used in the next step without further purification. MS obsd. (ESI⁺) [(M-NH₂)⁺]: 218.6.

### Intermediate Int-13

### 3-(3-phenyl-1,2,4-thiadiazol-5-yl)bicyclo[1.1.1]pentan-1-amine

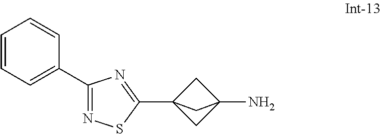 [SMILES: NC12CC(c3nc(-c4ccccc4)ns3)(C1)C2]

The title compound was prepared according to the following scheme:

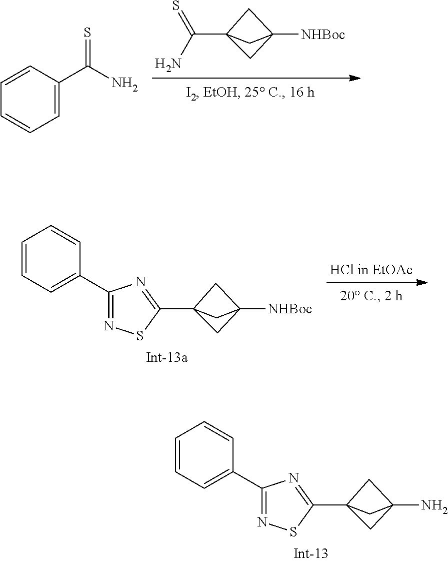 [SMILES: NC(=S)c1ccccc1 ; CC(C)(C)OC(=O)NC12CC(C(N)=S)(C1)C2 ; CC(C)(C)OC(=O)NC12CC(c3nc(-c4ccccc4)ns3)(C1)C2 ; NC12CC(c3nc(-c4ccccc4)ns3)(C1)C2]

### Step 1: Preparation of tert-butyl N-[3-(3-phenyl-1,2,4-thiadiazol-5-yl)-1-bicyclo[1.1.1]pentanyl]carbamate (Int-13a)

To a solution of tert-butyl N-(3-carbamothioyl-1-bicyclo[1.1.1]pentanyl)carbamate (200.0 mg, 0.83 mmol) in ethanol (10 mL) was added benzenecarbothioamide (566.15 mg, 4.13 mmol) and iodine (1675.73 mg, 6.6 mmol). After being stirred at 25° C. for 16 h, the reaction was quenched with saturated aqueous sodium thiosulfate solution (30 mL), followed by extraction with ethyl acetate (3×30 mL). The combined organic layer was dried over anhydrous Na₂SO₄ and concentrated under reduced pressure. The residue was purified by flash column (eluting with EtOAc/PE=35/65) to afford tert-butyl N-[3-(3-phenyl-1,2,4-thiadiazol-5-yl)-1-bicyclo[1.1.1]pentanyl]carbamate (Int-13a, 150 mg, 52.92%) as a white solid. MS obsd. (ESI⁺) [(M+H)⁺]: 344.1.

### Step 2: Preparation of 3-(3-phenyl-1,2,4-thiadiazol-5-yl)bicyclo[11.1.1]pentan-1-amine (Int-13)

To a solution of tert-butyl N-[3-(3-phenyl-1,2,4-thiadiazol-5-yl)-1-bicyclo[1.1.1]pentanyl]carbamate (Int-13a, 160.0 mg, 0.47 mmol) in EtOAc (5 mL) was added HCl in EtOAc (3 M, 7 mL). After being stirred at 20° C. for 2 h, the resulting solution was concentrated under reduced pressure to afford 3-(3-phenyl-1,2,4-thiadiazol-5-yl)bicyclo[1.1.1]pentan-1-amine (Int-13, 100 mg, HCl salt) as a white solid, which was used in the next step without further purification. MS obsd. (ESI⁺) [(M+H)⁺]: 489.1.

### Intermediate Int-14

### 3-[3-(4-chlorophenyl)-1,2,4-thiadiazol-5-yl]bicyclo[1.1.1]pentan-1-amine

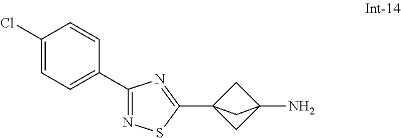 [SMILES: NC12CC(c3nc(-c4ccc(Cl)cc4)ns3)(C1)C2]

The title compound was prepared according to the following scheme:

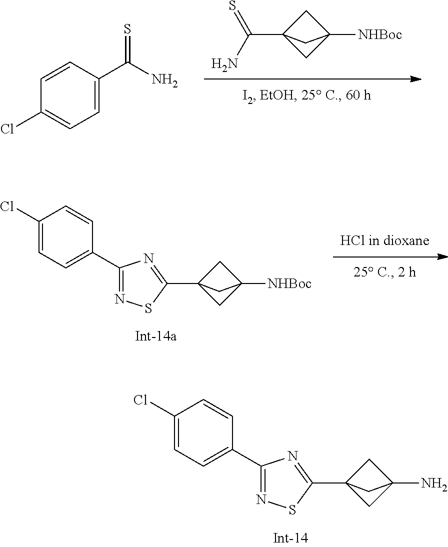 [SMILES: NC(=S)c1ccc(Cl)cc1 ; CC(C)(C)OC(=O)NC12CC(C(N)=S)(C1)C2 ; CC(C)(C)OC(=O)NC12CC(c3nc(-c4ccc(Cl)cc4)ns3)(C1)C2 ; NC12CC(c3nc(-c4ccc(Cl)cc4)ns3)(C1)C2]

### Step 1: Preparation of tert-butyl N-[3-[3-(4-chlorophenyl)-1,2,4-thiadiazol-5-yl]-1-bicyclo[1.1.1]pentanyl]carbamate (Int-14a)

To a solution of 4-chlorothiobenzamide (500.0 mg, 2.91 mmol) in ethanol (20 mL) were added tert-butyl N-(1-carbamothioyl-3-bicyclo[1.1.1]pentanyl)carbamate (850.61 mg, 3.51 mmol) and Iodine (738.59 mg, 2.91 mmol) at 25° C. After being stirring at 25° C. for 60 h, ethanol was removed under vacuum, and the residue was diluted with EtOAc (40 mL) and washed with water (2×20 mL) and brine (20 mL). The organic layer was dried over Na₂SO₄ and concentrated under reduced pressure to give tert-butyl N-[3-[3-(4-chlorophenyl)-1,2,4-thiadiazol-5-yl]-1-bicyclo[1.1.1]pentanyl]carbamate (Int-14a, 1.25 g, 91.16%) as a white solid. MS obsd. (ESI⁺) [(M+H-100)⁺]: 278.1.

### Step 2: Preparation of 3-[3-(4-chlorophenyl)-1,2,4-thiadiazol-5-yl]bicyclo[1.1.1]pentan-1-amine (Int-14)

A solution of tert-butyl N-[3-[3-(4-chlorophenyl)-1,2,4-thiadiazol-5-yl]-1-bicyclo[1.1.1]pentanyl]carbamate (Int-14a, 1.25 g, 3.31 mmol) in a solution of HCl in 1,4-dioxane (4 M, 20 mL) was stirred at 25° C. for 2 h. The solution was concentrated under reduced pressure to give 3-[3-(4-chlorophenyl)-1,2,4-thiadiazol-5-yl]bicyclo[1.1.1]pentan-1-amine (Int-14, 1 g, HCl salt) as a white solid, which was used in the next step without further purification. MS obsd. (ESI⁺) [(M+H)⁺]: 278.0.

### Intermediate Int-15

### 3-[5-(4-chlorophenyl)thiazol-2-yl]bicyclo[1.1.1]pentan-1-amine

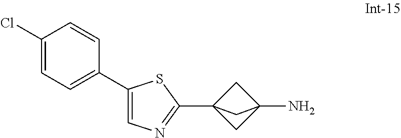 [SMILES: NC12CC(c3ncc(-c4ccc(Cl)cc4)s3)(C1)C2]

The title compound was prepared according to the following scheme:

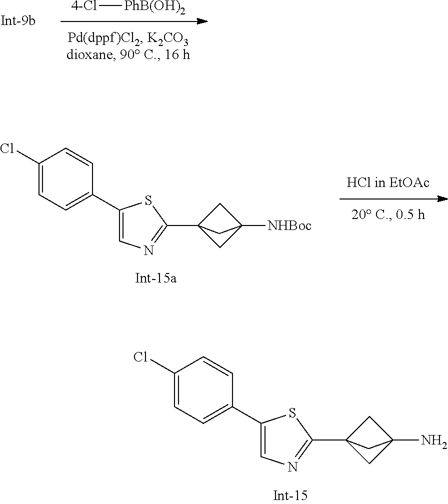

### Step 1: Preparation of tert-butyl N-[3-[5-(4-chlorophenyl)thiazol-2-yl]-1-bicyclo[1.1.1]pentanyl]carbamate (Int-15a)

To a solution of 4-chlorophenylboronic acid (45.29 mg, 0.29 mmol) and tert-butyl N-[3-(5-bromothiazol-2-yl)-1-bicyclo[1.1.1]pentanyl]carbamate (Int-9b, 100.0 mg, 0.29 mmol) in 1,4-dioxane (5 mL) were added potassium carbonate (120.09 mg, 0.87 mmol) and [1,1′-bis(diphenylphosphino)ferrocene]dichloropalladium (II) (21.19 mg, 0.03 mmol) at 25° C. under N₂ atmosphere. The reaction mixture was stirred at 90° C. for 16 h under nitrogen atmosphere. After being cooled to room temperature, the resulting solution was diluted with ethyl acetate (30 mL) and washed with brine (20 mL). The organic layer was dried over anhydrous sodium sulfate and concentrated under reduced pressure. The residue was purified by flash column (eluting with EtOAc/PE=30/70) to give tert-butyl N-[3-[5-(4-chlorophenyl)thiazol-2-yl]-1-bicyclo[1.1.1]pentanyl]carbamate (Int-15a, 62 mg, 56.8%) as a light brown solid. MS obsd. (ESI⁺) [(M+H)⁺]: 377.1.

### Step 4: Preparation of 3-[5-(4-chlorophenyl)thiazol-2-yl]bicyclo[1.1.1]pentan-1-amine (Int-15)

A solution of tert-butyl N-[3-[5-(4-chlorophenyl)thiazol-2-yl]-1-bicyclo[1.1.1]pentanyl]carbamate (Int-15a, 58.0 mg, 0.15 mmol) in a solution of HCl in EtOAc (4 M, 5 mL) was stirred at 25° C. for 30 min. The resulting solution was concentrated under reduced pressure to give 3-[5-(4-chlorophenyl)thiazol-2-yl]bicyclo[1.1.1]pentan-1-amine (Int-15, 40 mg, HCl salt) as a yellow solid, which was used in the next step without further purification. MS obsd. (ESI⁺) [(M+H)⁺]: 277.0.

### Intermediate Int-16

### 3-[5-(3,4-dichlorophenyl)thiazol-2-yl]bicyclo[1.1.1]pentan-1-amine

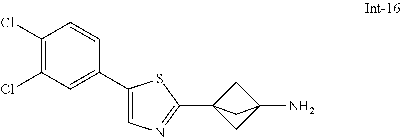 [SMILES: NC12CC(c3ncc(-c4ccc(Cl)c(Cl)c4)s3)(C1)C2]

The title compound was prepared in analogy to the procedure described for the preparation of 3-[5-(4-chlorophenyl)thiazol-2-yl]bicyclo[1.1.1]pentan-1-amine (Int-15), by using (3,4-dichlorophenyl)boronic acid instead of 4-chlorophenylboronic acid. MS obsd. (ESI⁺) [(M+H)⁺]: 311.1.

### Intermediate Int-17

### 3-(5-phenylthiazol-2-yl)bicyclo[11.1.1]pentan-1-amine

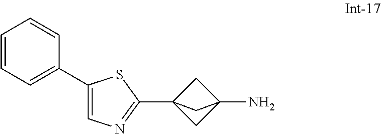 [SMILES: NC12CC(c3ncc(-c4ccccc4)s3)(C1)C2]

The title compound was prepared in analogy to the procedure described for the preparation of 3-(4-phenylthiazol-2-yl)bicyclo[1.1.1]pentan-1-amine (Int-9), by using tert-butyl N-[3-(5-bromothiazol-2-yl)-1-bicyclo[1.1.1]pentanyl]carbamate (Int-9b) to react with phenylboronic acid. MS obsd. (ESI⁺) [(M+H)⁺]: 243.0.

### Intermediate Int-18

### 3-(5-cyclopropylthiazol-2-yl)bicyclo[11.1.1]pentan-1-amine

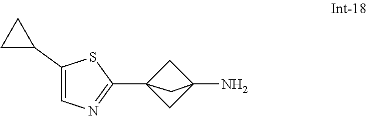 [SMILES: NC12CC(c3ncc(C4CC4)s3)(C1)C2]

The title compound was prepared in analogy to the procedure described for the preparation of 3-(4-phenylthiazol-2-yl)bicyclo[1.1.1]pentan-1-amine (Int-9), by using tert-butyl N-[3-(5-bromothiazol-2-yl)-1-bicyclo[1.1.1]pentanyl]carbamate (Int-9b) to react with cyclopropylboronic acid. MS obsd. (ESI⁺) [(M+H)⁺]: 207.1.

### Intermediate Int-19

### 3-(5-bromothiazol-2-yl)bicyclo[11.1.1]pentan-1-amine

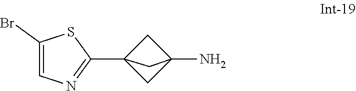 [SMILES: NC12CC(c3ncc(Br)s3)(C1)C2]

The title compound was prepared in analogy to the procedure described for the preparation of 3-(4-bromothiazol-2-yl)bicyclo[1.1.1]pentan-1-amine (Int-11), by using tert-butyl N-[3-(5-bromothiazol-2-yl)-1-bicyclo[1.1.1]pentanyl]carbamate (Int-9b) instead of tert-butyl N-[3-(4-bromothiazol-2-yl)-1-bicyclo[1.1.1]pentanyl]carbamate (Int-9c). MS obsd. (ESI⁺) [(M+H)⁺]: 247.3.

### Intermediate Int-20

### 3-(5-phenyl-1,2,4-thiadiazol-3-yl)bicyclo[1.1.1]pentan-1-amine

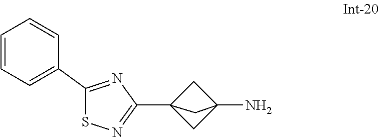 [SMILES: NC12CC(c3nsc(-c4ccccc4)n3)(C1)C2]

The title compound was prepared according to the following scheme:

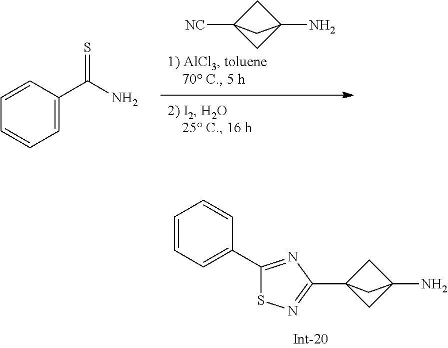 [SMILES: NC(=S)c1ccccc1 ; N#CC12CC(N)(C1)C2 ; NC12CC(c3nsc(-c4ccccc4)n3)(C1)C2]

To a solution of thiobenzamide (304.49 mg, 2.22 mmol) and 3-aminobicyclo[1.1.1]pentane-1-carbonitrile (240 mg, 2.22 mmol) in toluene (4.2 mL) was added aluminium chloride (295.93 mg, 2.22 mmol) at 25° C. After being stirred at 70° C. for 5 h under N₂ atmosphere, water (4.2 mL) and iodine (378.88 mg, 1.48 mmol) were added. The reaction mixture was stirred at 25° C. for 16 h under N₂ atmosphere. The resulting solution was adjusted to pH=9 with aqueous NaHCO₃ (5 mL, 15% w/w) solution and filtered by Celite. The filtrate was extracted with ethyl acetate (2×40 mL). The combined organic layer was washed with brine (30 mL), dried over Na₂SO₄ and concentrated under reduced pressure to give 3-(5-phenyl-1,2,4-thiadiazol-3-yl)bicyclo[1.1.1]pentan-1-amine (Int-20, 100 mg, 22.22%) as a brown solid. MS obsd. (ESI⁺) [(M+H)⁺]: 244.1.

### Intermediate Int-21

### 3-(2-phenylthiazol-4-yl)bicyclo[1.1.1]pentan-1-amine

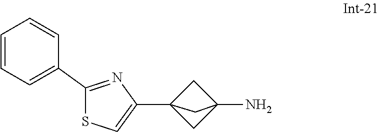 [SMILES: NC12CC(c3csc(-c4ccccc4)n3)(C1)C2]

The title compound was prepared according to the following scheme:

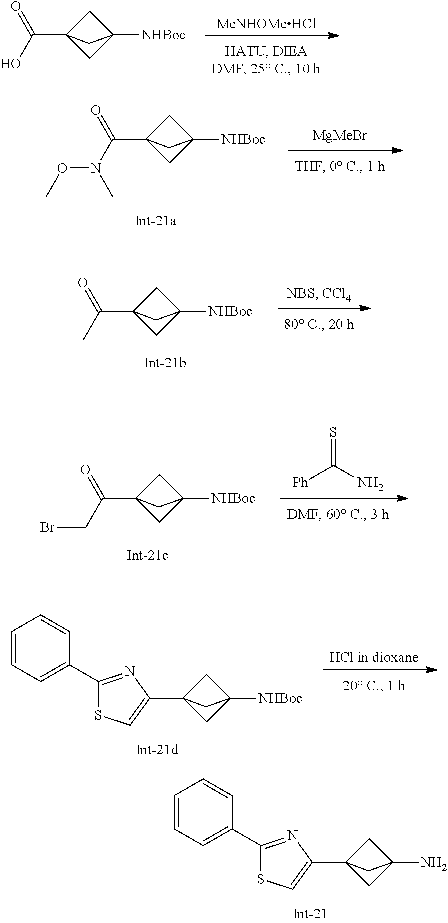

### Step 1: Preparation of tert-butyl N-[3-[methoxy(methyl)carbamoyl]-1-bicyclo[1.1.1]pentanyl]carbamate (Int-21a)

To a mixture of 3-(tert-butoxycarbonylamino)bicyclo[1.1.1]pentane-1-carboxylic acid (3.0 g, 13.2 mmol) in DMF (20 mL) were added HATU (5.5 g, 14.52 mmol), DIEA (5.1 g, 39.6 mmol) and N,O-dimethylhydroxylamine hydrochloride (1.55 g, 15.84 mmol). After being stirred at room temperature for 10 h, the resulting solution was diluted with H₂O (100 mL) and extracted with ethyl acetate (3×40 mL). The combined organic layer was dried over anhydrous sodium sulfate and concentrated under reduced pressure. The residue was purified by flash column (eluting with PE/EtOAc=50/50) to give tert-butyl N-[3-[methoxy(methyl)carbamoyl]-1-bicyclo[1.1.1]pentanyl]carbamate (Int-21a, 2.4 g, 67.5%) as a white solid. MS obsd. (ESI⁺) [(M+H)⁺]: 271.2.

### Step 2: Preparation of tert-butyl N-(3-acetyl-1-bicyclo[1.1.1]pentanyl)carbamate (Int-21b)

To a solution of tert-butyl N-[3-[methoxy(methyl)carbamoyl]-1-bicyclo[1.1.1]pentanyl]carbamate (Int-21a, 1.7 g, 6.29 mmol) in THF (17 mL) was added MeMgBr (6.29 mL, 18.87 mmol, 3 M in THF) dropwise at 0° C. under N₂ atmosphere. After being stirred at 0° C. for 1 h, the reaction was quenched with saturated aqueous NH₄Cl (20 mL). The aqueous layer was extracted with EtOAc (2×20 mL). The combined organic layer was washed with brine (20 mL), dried over Na₂SO₄, filtered and concentrated on a rotary evaporator. The residue was purified by flash column (eluting with PE/EtOAc=75/25) to give tert-butyl N-(3-acetyl-1-bicyclo[1.1.1]pentanyl)carbamate (Int-21b, 1.5 g, 90%) as a white solid. MS obsd. (ESI⁺) [(M+H-56)⁺]: 170.1. ¹H NMR (400 MHz, CDCl₃) δ ppm: 2.26 (s, 6H), 2.14 (s, 3H), 1.45 (s, 9H).

### Step 3: Preparation of tert-butyl N-[3-(2-bromoacetyl)-1-bicyclo[1.1.1]pentanyl]carbamate (Int-21c)

A solution of tert-butyl N-(3-acetyl-1-bicyclo[1.1.1]pentanyl)carbamate (Int-21b, 250.0 mg, 1.11 mmol), BPO (50.0 mg, 0.210 mmol) and NBS (237.5 mg, 1.33 mmol) in CCl₄ (10 mL) was heated to 80° C. and stirred for 20 h under N₂ temperature. After being cooled to room temperature, CCl₄ was removed under vacuum. The residue was dissolved into EtOAc (20 mL), washed with water (20 mL) and brine (20 mL), dried over Na₂SO₄ and concentrated under reduced pressure to give crude tert-butyl N-[3-(2-bromoacetyl)-1-bicyclo[1.1.1]pentanyl]carbamate (Int-21c, 200 mg, 58.9%) as a white solid, which was used for the next step without further purification. MS obsd. (ESI⁺) [(M+H-56)⁺]: 250.1.

### Step 4: Preparation of tert-butyl N-[3-(2-phenylthiazol-4-yl)-1-bicyclo[1.1.1]pentanyl]carbamate (Int-21d)

To a solution of tert-butyl N-[3-(2-bromoacetyl)-1-bicyclo[1.1.1]pentanyl]carbamate (Int-21c, 200 mg, 0.65 mmol) in DMF (10 mL) was added thiobenzamide (164.0 mg, 1.2 mmol). The reaction mixture was heated to 60° C. and stirred for 3 h. After being cooled to room temperature, the resulting solution was diluted with water (50 mL) and extracted with EtOAc (3×20 mL). The combined organic phase was washed with brine (20 mL), dried over anhydrous Na₂SO₄ and concentrated under reduced pressure. The residue was purified by flash column (eluting with PE/EtOAc=70/30) to give tert-butyl N-[3-(2-phenylthiazol-4-yl)-1-bicyclo[1.1.1]pentanyl]carbamate (Int-21d, 200 mg, 89.7%) as yellow solid. MS obsd. (ESI⁺) [(M+H)⁺]: 343.1.

### Step 5: Preparation of 3-(2-phenylthiazol-4-yl)bicyclo[1.1.1]pentan-1-amine (Int-21)

A solution of tert-butyl N-[3-(2-phenylthiazol-4-yl)-1-bicyclo[1.1.1]pentanyl]carbamate (Int-21d, 100.0 mg, 0.29 mmol) in a solution of HCl in 1,4-dioxane (4.0 M, 1.5 mL) was stirred at room temperature for 1 h. The resulting solution was concentrated under reduced pressure. The residue was purified by reversed flash column (eluting with H₂O/ACN=1/3) to afford 3-(2-phenylthiazol-4-yl)bicyclo[1.1.1]pentan-1-amine (Int-21, 65 mg, HCl salt, 80%) as a white solid. MS obsd. (ESI⁺) [(M+H)⁺]: 243.1.

### Intermediate Int-22

### 3-(2-phenylthiazol-4-yl)bicyclo[1.1.1]pentan-1-amine

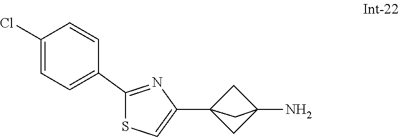 [SMILES: NC12CC(c3csc(-c4ccc(Cl)cc4)n3)(C1)C2]

The title compound was prepared in analogy to the procedure described for the preparation of 3-(2-phenylthiazol-4-yl)bicyclo[1.1.1]pentan-1-amine (Int-21), by using 4-chlorothiobenzamide instead of thiobenzamide. MS obsd. (ESI⁺) [(M+H)⁺]: 277.1.

### Intermediate Int-23

### 3-[5-(4-chlorophenyl)-1,3,4-oxadiazol-2-yl]bicyclo[1.1.1]pentan-1-amine

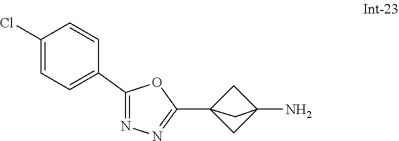 [SMILES: NC12CC(c3nnc(-c4ccc(Cl)cc4)o3)(C1)C2]

The title compound was prepared according to the following scheme:

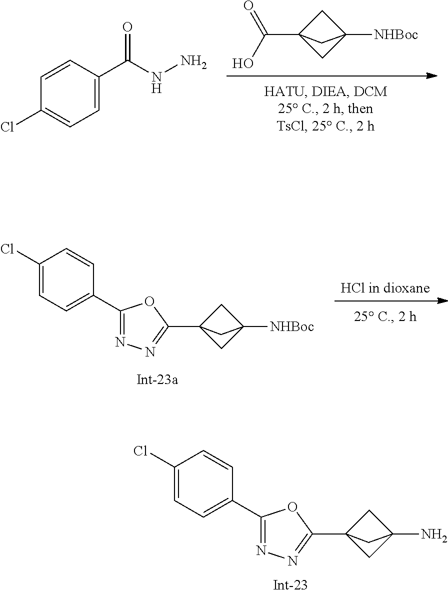 [SMILES: NNC(=O)c1ccc(Cl)cc1 ; CC(C)(C)OC(=O)NC12CC(C(=O)O)(C1)C2 ; CC(C)(C)OC(=O)NC12CC(c3nnc(-c4ccc(Cl)cc4)o3)(C1)C2 ; "HCl in dioxane" ; NC12CC(c3nnc(-c4ccc(Cl)cc4)o3)(C1)C2]

### Step 1: Preparation of tert-butyl N-[3-[5-(4-chlorophenyl)-1,3,4-oxadiazol-2-yl]-1-bicyclo[1.1.1]pentanyl]carbamate (Int-23a)

To a solution of HATU (245.17 mg, 0.64 mmol) in DCM (5 mL) were added 3-(tert-butoxycarbonylamino)bicyclo[1.1.1]pentane-1-carboxylic acid (133.21 mg, 0.59 mmol), 4-chlorobenzhydrazide (100.0 mg, 0.59 mmol) and DIEA (0.29 mL, 1.76 mmol). After being stirred at 25° C. for 2 h, TsCl (335.26 mg, 1.76 mmol) was added. The reaction mixture was stirred at 25° C. for 2 h. The resulting solution was diluted with water (60 mL) and extracted with DCM (2×20 mL). The combined organic phase was dried over anhydrous sodium sulfate and concentrated under reduced pressure. The residue was purified by flash column (eluting with EtOAc/PE=50/50) to give tert-butyl N-[3-[5-(4-chlorophenyl)-1,3,4-oxadiazol-2-yl]-1-bicyclo[1.1.1]pentanyl]carbamate (Int-23a, 190 mg, 85.34%) as a brown solid. MS obsd. (ESI⁺) [(M+H)⁺]: 380.0.

### Step 2: Preparation of 3-[5-(4-chlorophenyl)-1,3,4-oxadiazol-2-yl]bicyclo[1.1.1]pentan-1-amine (Int-23)

A solution of tert-butyl N-[3-[5-(4-chlorophenyl)-1,3,4-oxadiazol-2-yl]-1-bicyclo[1.1.1]pentanyl]carbamate (Int-23a, 190.0 mg, 0.53 mmol) in a solution of HCl in dioxane (7 M, 5.0 mL) was stirred at 25° C. for 2 h. The reaction was concentrated under reduced pressure to give 3-[5-(4-chlorophenyl)-1,3,4-oxadiazol-2-yl]bicyclo[1.1.1]pentan-1-amine (Int-23, 100 mg, HCl salt) as a white solid, which was used for the next step without further purification. MS obsd. (ESI⁺) [(M+H)⁺]: 262.0.

### Intermediate Int-24

### 3-[1-(4-chlorophenyl)triazol-4-yl]bicyclo[1.1.1]pentan-1-amine

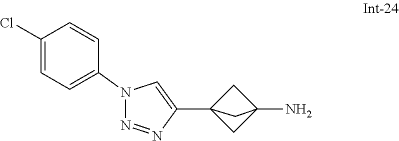 [SMILES: NC12CC(c3cn(-c4ccc(Cl)cc4)nn3)(C1)C2]

The title compound was prepared according to the following scheme:

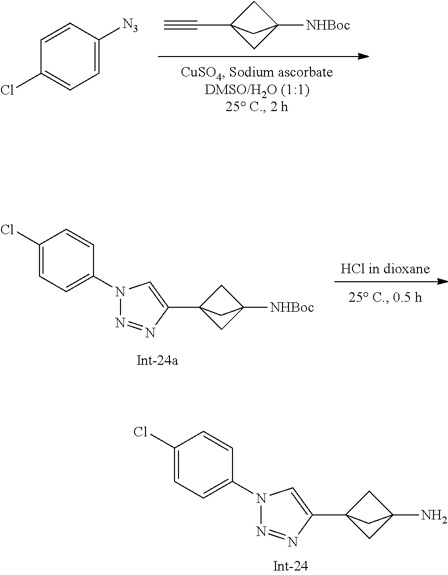

### p 1: Preparation of tert-butyl N-[3-[1-(4-chlorophenyl)triazol-4-yl]-1-bicyclo[1.1.1]pentanyl]carbamate (Int-24a)

A solution of 1-azido-4-chlorobenzene (460.5 mg, 3.0 mmol), tert-butyl N-(3-ethynyl-1-bicyclo[1.1.1]pentanyl)carbamate (207 mg, 1.0 mmol), sodium ascorbate (1188.7 mg, 6.0 mmol) and CuSO₄ (1248.4 mg, 5.0 mmol) in DMSO/H₂O (10 mL, V/V=50/50) was stirred at 25° C. for 2 h.

The resulting solution was diluted with ethyl acetate (180 mL) and washed with water (50 mL) and brine (50 mL), dried over Na₂SO₄ and concentrated under reduced pressure to give tert-butyl N-[3-[1-(4-chlorophenyl)triazol-4-yl]-1-bicyclo[1.1.1]pentanyl]carbamate (Int-24a, 180 mg, 50%) as a white solid, which was used for the next step without further purification. MS obsd. (ESI⁺) [(M+H)⁺]: m/z, 361.1.

### Step 2: Preparation of 3-[1-(4-chlorophenyl)triazol-4-yl]bicyclo[1.1.1]pentan-1-amine (Int-24)

A solution of tert-butyl N-[3-[1-(4-chlorophenyl)triazol-4-yl]-1-bicyclo[1.1.1]pentanyl]carbamate (Int-24a, 180 mg, 0.5 mmol) in a solution of HCl in 1,4-dioxane (4.0 M, 5 mL) was stirred at 25° C. for 30 min. The resulting solution was concentrated under reduced pressure to give 3-[1-(4-chlorophenyl)triazol-4-yl]bicyclo[1.1.1]pentan-1-amine (Int-24, HCl salt, 100 mg) as a white solid, which was used for the next step without further purification. MS obsd. (ESI⁺) [(M+H)⁺]: 261.1.

### Intermediate Int-25

### 3-[2-(4-chlorophenyl)triazol-4-yl]bicyclo[1.1.1]pentan-1-amine

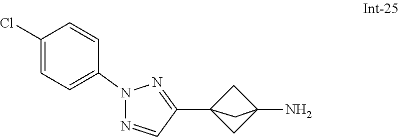 [SMILES: NC12CC(c3cnn(-c4ccc(Cl)cc4)n3)(C1)C2]

The title compound was prepared according to the following scheme:

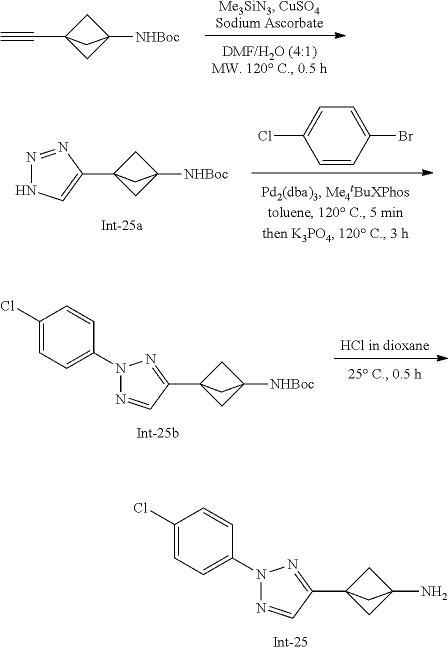

### Step 1: Preparation of tert-butyl N-[3-(1H-triazol-4-yl)-1-bicyclo[1.1.1]pentanyl]carbamate (Int-25a)

A solution of tert-butyl N-(3-ethynyl-1-bicyclo[1.1.1]pentanyl)carbamate (414 mg, 2 mmol), azidotrimethylsilane (276 mg, 2.4 mmol), sodium ascorbate (158.4 mg, 0.8 mmol) and CuSO₄ (25.0 mg, 0.1 mmol) in DMF/H₂O (8 mL, V/V=50/50) was stirred at 120° C. for 30 min under microwave irradiation. After being cooled to room temperature, the resulting solution was diluted with ethyl acetate (80 mL) and washed with water (20 mL) and brine (20 mL), dried over Na₂SO₄ and concentrated under reduced pressure. The residue was purified by flash column (eluting with PE/EtOAc=75/25) to give tert-butyl N-[3-(1H-triazol-4-yl)-1-bicyclo[1.1.1]pentanyl]carbamate (Int-25a, 400 mg, 80%) as a white solid. MS obsd. (ESI⁺) [(M+H)⁺]: 251.1.

### Step 2: Preparation of tert-butyl N-[3-[2-(4-chlorophenyl)triazol-4-yl]-1-bicyclo[1.1.1]pentanyl]carbamate (Int-25b)

A flame-dried flask was equipped with a magnetic stir bar and charged with Pd₂(dba)₃ (18.3 mg, 0.02 mmol) and 2-di-tert-butylphosphino-3,4,5,6-tetramethyl-2′,4′,6′-triisopropyl-1,1′-biphenyl (19.2 mg, 0.04 mmol, Sigma-Aldrich, #675938). The flask was evacuated and backfilled with N₂. Toluene (5 mL) was added, and the resulting mixture was stirred for 3 min at 120° C. until the color turned from dark-purple to dark-brown. A second, previously dried flask was equipped with a stir bar and charged with K₃PO₄ (678.4 mg, 3.2 mmol) and tert-butyl N-[3-(1H-triazol-4-yl)-1-bicyclo[1.1.1]pentanyl]carbamate (Int-25a, 400 mg, 1.6 mmol). The flask was evacuated and backfilled with N₂. The 1-bromo-4-chlorobenzene (305.6 mg, 1.6 mmol) was then added as well as the premixed catalyst solution and additional toluene (5 mL). After being heated at 120° C. for 3 h. The reaction was cooled down to 25° C. and diluted with EtOAc (10 mL). The organic layer was washed with brine, dried over Mg₂SO₄, filtered, and evaporated. The residue was purified by flash column (eluting with PE/EtOAc=80/20) to give tert-butyl N-[3-[2-(4-chlorophenyl)triazol-4-yl]-1-bicyclo[1.1.1]pentanyl]carbamate (Int-25b, 115.2 mg, 20%) as a white solid. MS obsd. (ESI⁺) [(M+H)⁺]: 361.1.

### Step 3: Preparation of 3-[2-(4-chlorophenyl)triazol-4-yl]bicyclo[1.1.1]pentan-1-amine (Int-25)

A solution of tert-butyl N-[3-[2-(4-chlorophenyl)triazol-4-yl]-1-bicyclo[1.1.1]pentanyl]carbamate (Int-25b, 100 mg, 0.28 mmol) in a solution of HCl in 1,4-dioxane (4.0 M, 5 mL) was stirred at 25° C. for 30 min. The resulting solution was concentrated under reduced pressure to give 3-[2-(4-chlorophenyl)triazol-4-yl]bicyclo[1.1.1]pentan-1-amine (Int-25, 70 mg, HCl salt) as a white solid, which was used for the next step without further purification. MS obsd. (ESI⁺) [(M+H)⁺]: 261.1.

### Intermediate Int-26

### 3-[3-(4-chlorophenyl)-1,2,4-oxadiazol-5-yl]bicyclo[1.1.1]pentan-1-amine

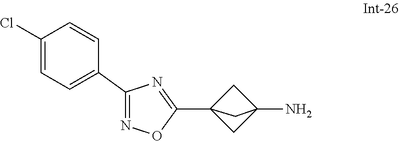 [SMILES: NC12CC(c3nc(-c4ccc(Cl)cc4)no3)(C1)C2]

The title compound was prepared according to the following scheme:

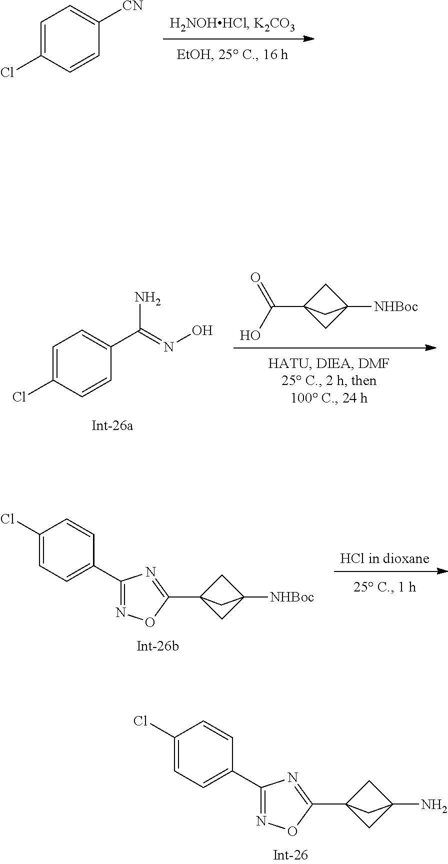

### Step 1: Preparation of 4-chloro-N′-hydroxy-benzamidine (Int-26a)

To a solution of 4-chlorobenzonitrile (10.0 g, 72.69 mmol) in ethanol (100 mL) was added hydroxylamine hydrochloride (10.1 g, 145.38 mmol) and K₂CO₃ (20.09 g, 145.38 mmol) at 25° C. The reaction was stirred at 25° C. for 16 h. After filtration, the filtrate was concentrated under reduced pressure to give 4-chloro-N-hydroxy-benzamidine (Int-26a, 15.25 g, 100%) as a white solid, which was used in next step without purification. MS obsd. (ESI⁺) [(M+H)⁺]: 171.0.

### Step 2: Preparation of tert-butyl N-[3-[3-(4-chlorophenyl)-1,2,4-oxadiazol-5-yl]-1-bicyclo[1.1.1]pentanyl]carbamate (Int-26b)

To a solution of 3-(tert-butoxycarbonylamino)bicyclo[1.1.1]pentane-1-carboxylic acid (1.20 g, 5.28 mmol) in DMF (13 mL) were added HATU (2.0 g, 5.28 mmol), DIEA (2.18 mL, 13.19 mmol) and 4-chloro-N-hydroxy-benzamidine (Int-26a, 0.75 g, 4.40 mmol). The reaction was stirred at 25° C. for 2 h and then stirred at 100° C. for 24 h. After being cooled to room temperature, the mixture was diluted with EtOAc (40 mL) and washed with water (2×20 mL) and brine (20 mL). The organic layer was dried over Na₂SO₄ and concentrated under reduced pressure. The residue was purified by flash column (eluting with PE/EA=50/50) to give tert-butyl N-[3-[3-(4-chlorophenyl)-1,2,4-oxadiazol-5-yl]-1-bicyclo[1.1.1]pentanyl]carbamate (Int-26b, 1.0 g, 63%), which was used for the next step without further purification. MS obsd. (ESI⁺) [(M+H)⁺]: 262.1.

### Step 3: Preparation of 3-[3-(4-chlorophenyl)-1,2,4-oxadiazol-5-yl]bicyclo[1.1.1]pentan-1-amine (Int-26)

A solution of tert-butyl N-[3-[3-(4-chlorophenyl)-1,2,4-oxadiazol-5-yl]-1-bicyclo[1.1.1]pentanyl]carbamate (Int-26b, 100.0 mg, 0.28 mmol) in a solution of HCl in 1,4-dioxane (7 M, 2.0 mL) was stirred at ambient temperature for 1 h. The reaction was concentrated under reduced pressure to give 3-[3-(4-chlorophenyl)-1,2,4-oxadiazol-5-yl]bicyclo[1.1.1]pentan-1-amine (Int-26, 73 mg, HCl salt) as a white solid which was used in next step directly without further purification. MS obsd. (ESI⁺) [(M+H)⁺]: 262.0.

### Intermediate Int-27

### 3-[5-(4-chlorophenyl)-1,2,4-oxadiazol-3-yl]bicyclo[1.1.1]pentan-1-amine

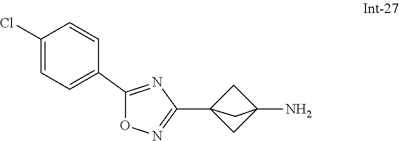 [SMILES: NC12CC(c3noc(-c4ccc(Cl)cc4)n3)(C1)C2]

The title compound was prepared according to the following scheme:

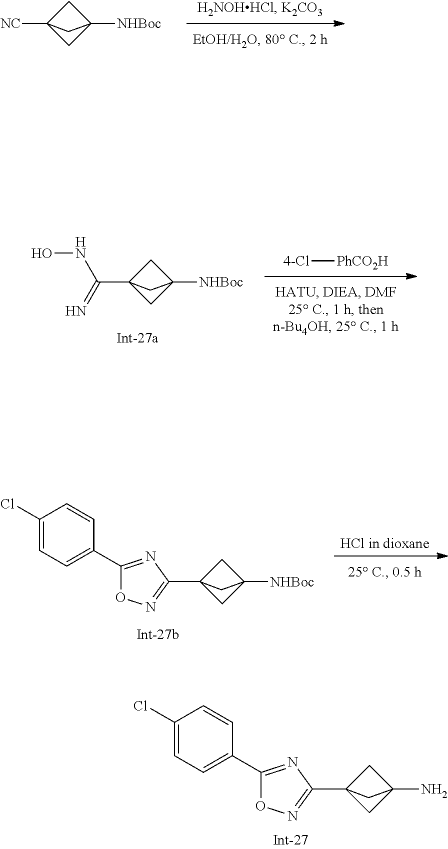

### Step 1: Preparation of tert-butyl N-[3-(N-hydroxycarbamimidoyl)-1-bicyclo[1.1.1]pentanyl]carbamate (Int-27a)

To a 50 mL of round-bottom flask was charged with tert-butyl N-(3-cyano-1-bicyclo[1.1.1]pentanyl)carbamate (750.0 mg, 3.6 mmol), sodium bicarbonate (605.08 mg, 7.2 mmol) and hydroxylamine hydrochloride (500 mg, 7.2 mmol) in ethanol (15 mL) and water (4 mL). The reaction was stirred at 80° C. for 1 h. After removing the solvent under vacuum, the residue was diluted with water (10 mL) and extracted with EtOAc (3×20 mL). The combined organic layer was washed with brine (40 mL), dried over Na₂SO₄, filtered and concentrated on a rotary evaporator to give tert-butyl N-[3-(N-hydroxycarbamimidoyl)-1-bicyclo[1.1.1]pentanyl]carbamate (Int-27a, 800 mg, 87.5%) as a light yellow solid, which was used directly for the next step. MS obsd. (ESI⁺) [(M+H)⁺]: 242.2.

### Step 2: Preparation of tert-butyl N-[3-[5-(4-chlorophenyl)-1,2,4-oxadiazol-3-yl]-1-bicyclo[1.1.1]pentanyl]carbamate (Int-27b)

To a solution of 4-chlorobenzoic acid (486.65 mg, 3.11 mmol) in DMF (15 mL) were added HATU (1300 mg, 3.42 mmol), TEA (1.3 mL, 9.32 mmol) and tert-butyl N-[3-(N-hydroxycarbamimidoyl)-1-bicyclo[1.1.1]pentanyl]carbamate (Int-27a, 750.0 mg, 3.11 mmol) at room temperature. The reaction was stirred for 1 h, followed by the addition of tetrabutylammonium hydroxide (1.2 mL, 4.6 mmol). After being stirred for another 1 h, the resulting solution was diluted with water (150 mL) and extracted with EtOAc (3×40 mL). The combined organic layer was washed with brine (40 mL), dried over Na₂SO₄, filtered and concentrated on a rotary evaporator. The residue was purified by flash column (eluting with PE/EtOAc=70/30) to give tert-butyl N-[3-[5-(4-chlorophenyl)-1,2,4-oxadiazol-3-yl]-1-bicyclo[1.1.1]pentanyl]carbamate (Int-27b, 510 mg, 45%) as a white solid. MS obsd. (ESI⁺) [(M+H)⁺]: 362.2. ¹H NMR (400 MHz, CDCl₃) δ ppm: 7.99 (d, J=8.6 Hz, 2H), 7.43 (d, J=8.6 Hz, 2H), 2.42 (s, 6H), 1.40 (s, 9H).

### Step 3: Preparation of 3-[5-(4-chlorophenyl)-1,2,4-oxadiazol-3-yl]bicyclo[1.1.1]pentan-1-amine (Int-27)

A solution of tert-butyl N-[3-[5-(4-chlorophenyl)-1,2,4-oxadiazol-3-yl]-1-bicyclo[1.1.1]pentanyl]carbamate (Int-27b, 510 mg, 1.41 mmol) in a solution of HCl in dioxane (4.0 M, 10.0 mL) was stirred at ambient temperature for 30 min. The resulting solution was concentrated under reduced pressure to afford 3-[5-(4-chlorophenyl)-1,2,4-oxadiazol-3-yl]bicyclo[1.1.1]pentan-1-amine (Int-27, 368 mg, HCl salt) as a white solid, which was used directly for the next step. MS obsd. (ESI⁺) [(M+H)⁺]: 262.1.

### Intermediate Int-28

### 3-[1-(4-chlorophenyl)pyrazol-3-yl]bicyclo[1.1.1]pentan-1-amine

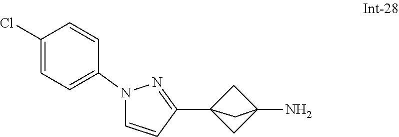 [SMILES: NC12CC(c3ccn(-c4ccc(Cl)cc4)n3)(C1)C2]

The title compound was prepared according to the following scheme:

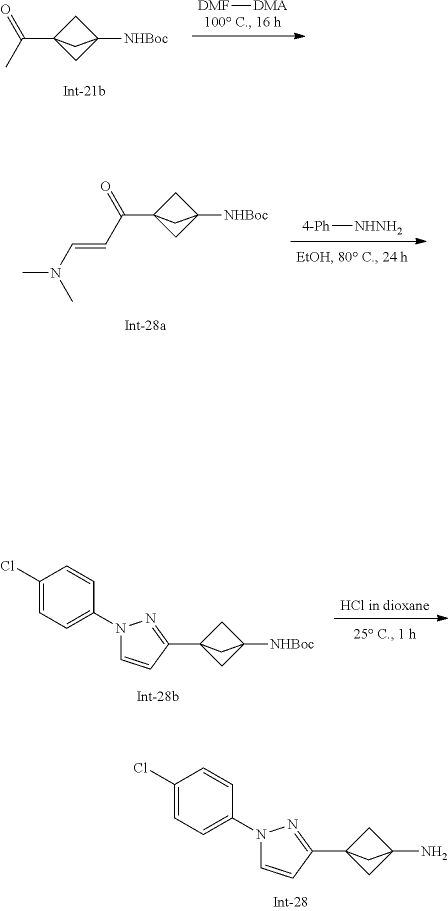

### Step 1: Preparation of tert-butyl N-[3-[(E)-3-(dimethylamino)prop-2-enoyl]-1-bicyclo[1.1.1]pentanyl]carbamate (Int-28a)

A solution of tert-butyl N-(3-acetyl-1-bicyclo[1.1.1]pentanyl)carbamate (Int-21b, 1.0 g, 4.44 mmol) in DMF-DMA (15.0 mL, 4.44 mmol) was heated with stirring at 100° C. for 16 h. After being cooled down to room temperature, the mixture was diluted with EtOAc (40 mL), washed with water (2×20 mL) and brine (20 mL). The organic layer was dried over Na₂SO₄, concentrated in vacuum to give crude tert-butyl N-[3-[(E)-3-(dimethylamino)prop-2-enoyl]-1-bicyclo[1.1.1]pentanyl]carbamate (Int-28a, 1.23 g, 91.92%), which was used for the next step without further purification. MS obsd. (ESI⁺) [(M+H)⁺]: 281.1.

### Step 2: Preparation of tert-butyl N-[3-[1-(4-chlorophenyl)pyrazol-3-yl]-1-bicyclo[1.1.1]pentanyl]carbamate (Int-28b)

To a solution of tert-butyl N-[3-[(E)-3-(dimethylamino)prop-2-enoyl]-1-bicyclo[1.1.1]pentanyl]carbamate (Int-28a, 1.23 g, 4.39 mmol) in ethanol (30 mL) was added 4-chlorophenylhydrazine hydrochloride (1.59 g, 8.91 mmol) at 25° C. The resulting mixture was stirred at 80° C. for 24 h. The solvent was removed in vacuum, the residue was diluted with EtOAc (40 mL) and washed with water (2×20 mL) and brine (20 mL). The organic layer was dried over Na₂SO₄ and concentrated under reduced pressure. The residue was purified by flash column (eluting with PE/EA=50/50) to give tert-butyl N-[3-[1-(4-chlorophenyl)pyrazol-3-yl]-1-bicyclo[1.1.1]pentanyl]carbamate (Int-28b, 1.16 g, 73.48%). MS obsd. (ESI⁺) [(M+H)⁺]: 360.14.

### Step 3: Preparation of 3-[1-(4-chlorophenyl)pyrazol-3-yl]bicyclo[1.1.1]pentan-1-amine (Int-28)

A solution of tert-butyl N-[3-[1-(4-chlorophenyl)pyrazol-3-yl]-1-bicyclo[1.1.1]pentanyl]carbamate (Int-28b, 88.16 mg, 0.25 mmol) in a solution of HCl in 1,4-dioxane (4 M, 4.0 mL, 16 mmol) was stirred at ambient temperature for 1 h. The resulting solution was concentrated under reduced pressure to give 3-[1-(4-chlorophenyl)pyrazol-3-yl]bicyclo[1.1.1]pentan-1-amine (Int-28, 63.5 mg, HCl salt) as a white solid, which was used for the next step without further purification. MS obsd. (ESI⁺) [(M+H)⁺]: 260.0.

### Intermediate Int-29

### 3-[2-(4-chlorophenyl)tetrazol-5-yl]bicyclo[1.1.1]pentan-1-amine

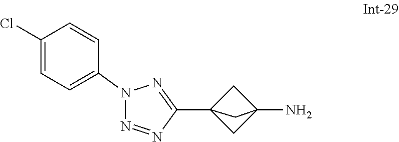 [SMILES: NC12CC(c3nnn(-c4ccc(Cl)cc4)n3)(C1)C2]

The title compound was prepared according to the following scheme:

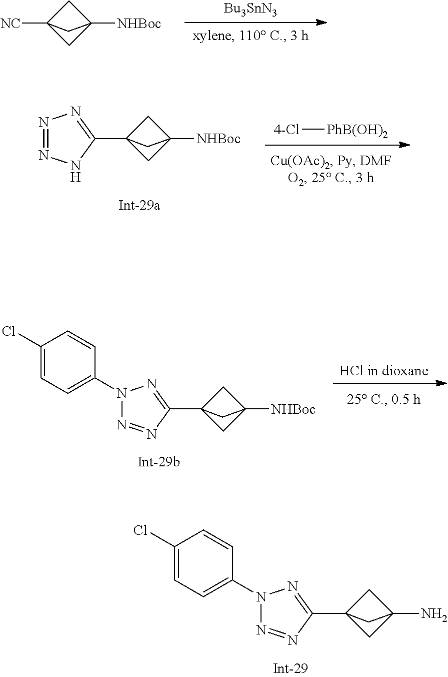

### Step 1: Preparation of tert-butyl N-[3-(1H-tetrazol-5-yl)-1-bicyclo[1.1.1]pentanyl]carbamate (Int-29a)

A solution of tert-butyl N-(3-cyano-1-bicyclo[1.1.1]pentanyl)carbamate (1.0 g, 4.8 mmol) and Bu₃SnN₃ (3.18 g, 9.6 mmol) in xylene (15 mL) was heated to 110° C. and stirred for 3 h under N₂. After being cooled to room temperature, the mixture was diluted with EtOAc (20 mL) and washed with water (20 mL) and brine (20 mL), dried over Na₂SO₄ and concentrated under reduced pressure to give tert-butyl N-[3-(1H-tetrazol-5-yl)-1-bicyclo[1.1.1]pentanyl]carbamate (Int-29a, 900 mg, 94.7%) as yellow liquid, which was used for the next step without further purification. MS obsd. (ESI⁺) [(M+H-56)⁺]: 196.1.

### Step 2: Preparation of tert-butyl N-[3-[2-(4-chlorophenyl)tetrazol-5-yl]-1-bicyclo[1.1.1]pentanyl]carbamate (Int-29b)

A solution of tert-butyl N-[3-(1H-tetrazol-5-yl)-1-bicyclo[1.1.1]pentanyl]carbamate (Int-29a, 500 mg, 2 mmol), Cu(OAc)₂ (726.4 mg, 4 mmol), pyridine (448.0 mg, 4 mmol) and (4-chlorophenyl)boronic acid (624.0 mg, 4 mmol) in DMF (5 mL) was stirred at 25° C. for 3 h under 02 atmosphere. The reaction mixture was diluted with EtOAc (20 mL) and washed with water (30 mL) and brine (20 mL), dried over Na₂SO₄ and concentrated under reduced pressure. The residue was purified by flash column (eluting with PE/EtOAc=80/20) to give tert-butyl N-[3-[2-(4-chlorophenyl)tetrazol-5-yl]-1-bicyclo[1.1.1]pentanyl]carbamate (Int-29b, 360.5 mg, 50%) as a white solid. MS obsd. (ESI⁺) [(M+H)⁺]: 362.1.

### Step 3: Preparation of 3-[2-(4-chlorophenyl)tetrazol-5-yl]bicyclo[1.1.1]pentan-1-amine (Int-29)

A solution of tert-butyl N-[3-[2-(4-chlorophenyl)tetrazol-5-yl]-1-bicyclo[1.1.1]pentanyl]carbamate (Int-29b, 110 mg, 0.28 mmol) in a solution of HCl in 1,4-dioxane (4.0 M, 5 mL) was stirred at 25° C. for 30 min. The reaction mixture was concentrated under reduced pressure to give 3-[2-(4-chlorophenyl)tetrazol-5-yl]bicyclo[1.1.1]pentan-1-amine (Int-29, 78 mg, HCl salt) as a white solid, which was used for the next step without further purification. MS obsd. (ESI⁺) [(M+H)⁺]: m/z, 262.1.

### Intermediate Int-30

### 3-[4-(4-chlorophenyl)pyrimidin-2-yl]bicyclo[1.1.1]pentan-1-amine

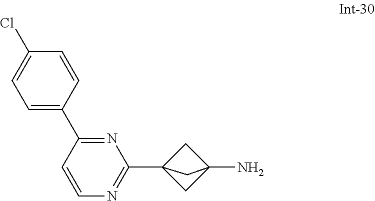 [SMILES: NC12CC(c3nccc(-c4ccc(Cl)cc4)n3)(C1)C2]

The title compound was prepared according to the following scheme:

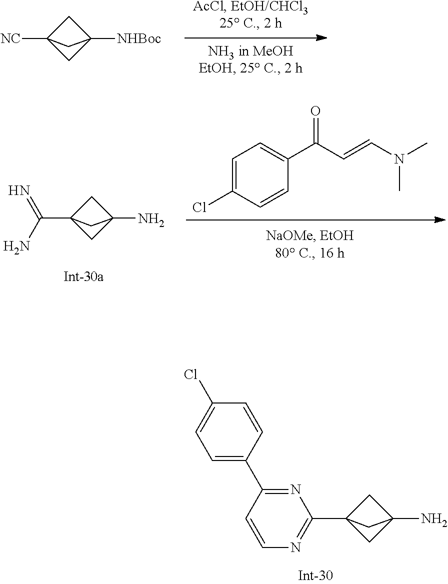 [SMILES: "AcCl, EtOH/CHCl325° C., 2 h" ; CC(C)(C)OC(=O)NC12CC(C#N)(C1)C2 ; "NH3 in MeOHEtOH, 25° C., 2 h" ; N=C(N)C12CC(N)(C1)C2 ; CN(C)/C=C/C(=O)c1ccc(Cl)cc1 ; NC12CC(c3nccc(-c4ccc(Cl)cc4)n3)(C1)C2]

### Step 1: Preparation of 3-aminobicyclo[1.1.1]pentane-1-carboxamidine (Int-30a)

To a solution of tert-butyl N-(3-cyano-1-bicyclo[1.1.1]pentanyl)carbamate (500.0 mg, 2.4 mmol) in a mixture of chloroform (15 mL) and ethanol (20 mL) was added acetyl chloride (3.41 mL, 48.02 mmol). After being stirred at 25° C. for 2 h, the solvent was removed under vacuum, the residue was dissolved in a solution of NH₃ in MeOH (10 mL, 4 M) and stirred at 25° C. for further 2 h. The resulting solution was concentrated under reduced pressure to afford 3-aminobicyclo[1.1.1]pentane-1-carboxamidine (Int-30a, 250 mg, 83.19%) as a white solid, which was used for next step without further purification. MS obsd. (ESI⁺) [(M+H)⁺]: 126.2.

### Step 1: Preparation of 3-[4-(4-chlorophenyl)pyrimidin-2-yl]bicyclo[1.1.1]pentan-1-amine (Int-30)

To a solution of (E)-1-(4-chlorophenyl)-3-(dimethylamino)prop-2-en-1-one (335.02 mg, 1.6 mmol) in ethanol (10 mL) were added 3-aminobicyclo[1.1.1]pentane-1-carboxamidine (Int-30a, 200.0 mg, 1.6 mmol) and sodium methanolate (172.63 mg, 3.2 mmol). The mixture was stirred at 80° C. for 16 h. After being cooled to room temperature, the resulting solution was concentrated under reduced pressure. The residue was purified by reversed flash column (eluting with ACN/H₂O=50/50) to afford 3-[4-(4-chlorophenyl)pyrimidin-2-yl]bicyclo[1.1.1]pentan-1-amine (Int-30, 150 mg, 34.55%) as a red solid. MS obsd. (ESI⁺) [(M+H)⁺]: 272.1.

### Intermediate Int-31

### 3-[2-(4-chlorophenyl)pyrimidin-4-yl]bicyclo[1.1.1]pentan-1-amine

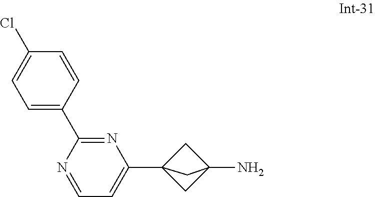 [SMILES: NC12CC(c3ccnc(-c4ccc(Cl)cc4)n3)(C1)C2]

The title compound was prepared according to the following scheme:

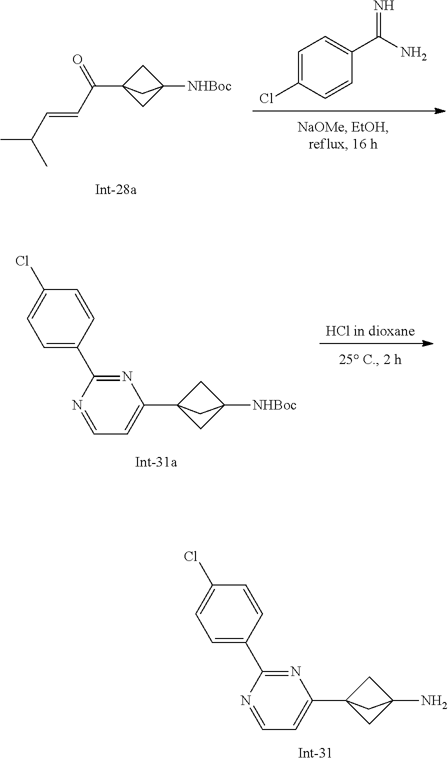 [SMILES: CC(C)/C=C/C(=O)C12CC(NC(=O)OC(C)(C)C)(C1)C2 ; N=C(N)c1ccc(Cl)cc1 ; CC(C)(C)OC(=O)NC12CC(c3ccnc(-c4ccc(Cl)cc4)n3)(C1)C2 ; "HCl in dioxane" ; NC12CC(c3ccnc(-c4ccc(Cl)cc4)n3)(C1)C2]

### ep 1: Preparation of tert-butyl N-[3-[2-(4-chlorophenyl)pyrimidin-4-yl]-1-bicyclo[1.1.1]pentanyl]carbamate (Int-31a)

To a solution of tert-butyl N-[3-[(E)-3-(dimethylamino)prop-2-enoyl]-1-bicyclo[1.1.1]pentanyl]carbamate (Int-28a, 1.1 g, 3.33 mmol) and 4-chlorobenzene-1-carboximidamide hydrochloride (1911.55 mg, 10 mmol) in ethanol (30 mL) was added NaOMe (540.27 mg, 10 mmol) under nitrogen atmosphere. The reaction mixture was refluxed for 16 h and then concentrated under reduced pressure. The residue was purified by flash column (eluting with PE/EtOAc=70/30) to give tert-butyl N-[3-[2-(4-chlorophenyl)pyrimidin-4-yl]-1-bicyclo[1.1.1]pentanyl]carbamate (Int-31a, 780 mg, 56.61%) as a white solid. MS obsd. (ESI⁺) [(M+H)⁺]: 372.2.

### Step 1: Preparation of 3-[2-(4-chlorophenyl)pyrimidin-4-yl]bicyclo[1.1.1]pentan-1-amine (Int-31)

A solution of tert-butyl N-[3-[2-(4-chlorophenyl)pyrimidin-4-yl]-1-bicyclo[1.1.1]pentanyl]carbamate (Int-31a, 780.0 mg, 1.89 mmol) in a solution of HCl in dioxane (4.0 M, 15.0 mL, 60 mmol) was stirred at room temperature for 2 h. The resulting solution was concentrated under reduced pressure to give 3-[2-(4-chlorophenyl)pyrimidin-4-yl]bicyclo[1.1.1]pentan-1-amine (Int-31, 600 mg, HCl salt) as a white solid. MS obsd. (ESI⁺) [(M+H)⁺]: 272.1.

### Intermediate Int-32

### 3-[3-(4-fluorophenyl)-1,2,4-oxadiazol-5-yl]bicyclo[1.1.1]pentan-1-amine

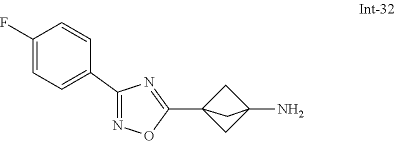 [SMILES: NC12CC(c3nc(-c4ccc(F)cc4)no3)(C1)C2]

The title compound was prepared in analogy to the procedure described for the preparation of 3-[3-(4-chlorophenyl)-1,2,4-oxadiazol-5-yl]bicyclo[1.1.1]pentan-1-amine (Int-26), by using 4-fluorobenzonitrile instead of 4-chlorobenzonitrile. MS obsd. (ESI⁺) [(M+H)⁺]: 246.1.

### Intermediate Int-33

### 3-[5-bromo-2-(4-chlorophenyl)triazol-4-yl]bicyclo[1.1.1]pentan-1-amine

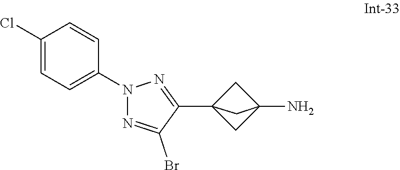 [SMILES: NC12CC(c3nn(-c4ccc(Cl)cc4)nc3Br)(C1)C2]

The title compound was prepared according to the following scheme:

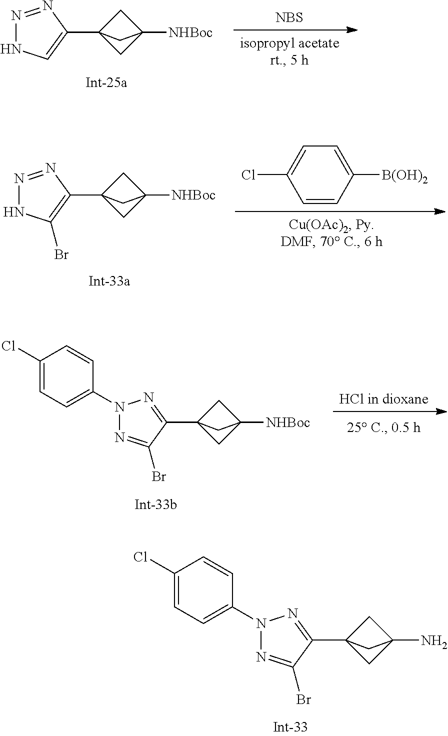

### Step 1: Preparation of tert-butyl N-[3-(5-bromo-1H-triazol-4-yl)-1-bicyclo[1.1.1]pentanyl]carbamate (Int-33a)

To a mixture of tert-butyl N-[3-(1H-triazol-4-yl)-1-bicyclo[1.1.1]pentanyl]carbamate (Int-25a, 500 mg, 2.0 mmol) in isopropyl acetate (30 ml) was added NBS (1068 mg, 6.0 mmol). After being stirred at rt for 5 h, the reaction was extracted with ethyl acetate (3×20 mL) and washed with water (10 mL) and brine (10 mL), dry over Na₂SO₄ and concentrated under reduced pressure. The residue was purified by flash column (eluting with EtOAc/PE=20/80) to give tert-butyl N-[3-(5-bromo-1H-triazol-4-yl)-1-bicyclo[1.1.1]pentanyl]carbamate (Int-33a, 480 mg, 73.17%) as a white solid, which was used for the next step without further purification. MS obsd. (ESI⁺) [(M+H)⁺]: 329.1.

### Step 2: Preparation of tert-butyl N-[3-[5-bromo-2-(4-chlorophenyl)triazol-4-yl]-1-bicyclo[1.1.1]pentanyl]carbamate (Int-33b)

To a mixture of tert-butyl N-[3-(5-bromo-1H-triazol-4-yl)-1-bicyclo[1.1.1]pentanyl]carbamate (Int-33a, 400 mg, 1.22 mmol) in DMF (20 ml) was added (4-chlorophenyl)boronic acid (380.6 mg, 2.44 mmol), Cu(OAc)₂ (443.2 mg, 2.44 mmol) and pyridine (289.5 mg, 3.66 mmol). After being stirred at 70° C. for 6 h, the reaction was extracted with ethyl acetate (3×20 mL) and washed with water (10 mL) and brine (10 mL). The organic layer was dried over Na₂SO₄ and concentrated under reduced pressure. The residue was purified by flash column (eluting with EtOAc/PE=20/80) to afford tert-butyl N-[3-[5-bromo-2-(4-chlorophenyl)triazol-4-yl]-1-bicyclo[1.1.1]pentanyl]carbamate (Int-33b, 107 mg, 20%) as a white solid. MS obsd. (ESI⁺) [(M+H-56)*]: 383.1. ¹H NMR (400 MHz, CDCl₃) δ ppm: 7.93 (d, J=8.8 Hz, 2H), 7.42 (d, J=8.8 Hz, 2H), 5.06 (s, 1H), 2.51 (s, 6H), 1.48 (s, 9H).

### Step 3: Preparation of 3-[5-bromo-2-(4-chlorophenyl)triazol-4-yl]bicyclo[1.1.1]pentan-1-amine (Int-33)

tert-Butyl N-[3-[5-bromo-2-(4-chlorophenyl)triazol-4-yl]-1-bicyclo[1.1.1]pentanyl]carbamate (Int-33b, 100 mg, 0.23 mmol) was placed in a round-bottomed flask, followed by the addition of a solution of HCl in 1,4-dioxane (4.0 M, 5 mL). After being stirred at rt for 0.5 h, the reaction mixture was concentrated under reduced pressure to give 3-[5-bromo-2-(4-chlorophenyl)triazol-4-yl]bicyclo[1.1.1]pentan-1-amine (Int-33, 50 mg, HCl salt) as yellow oil, which was used for the next step without further purification. MS obsd. (ESI⁺) [(M+H)⁺]: 339.1.

### Intermediate Int-34

### 3-[2-(4-chlorophenyl)-5-methyl-triazol-4-yl]bicyclo[1.1.1]pentan-1-amine

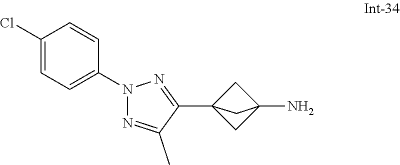 [SMILES: Cc1nn(-c2ccc(Cl)cc2)nc1C12CC(N)(C1)C2]

The title compound was prepared according to the following scheme:

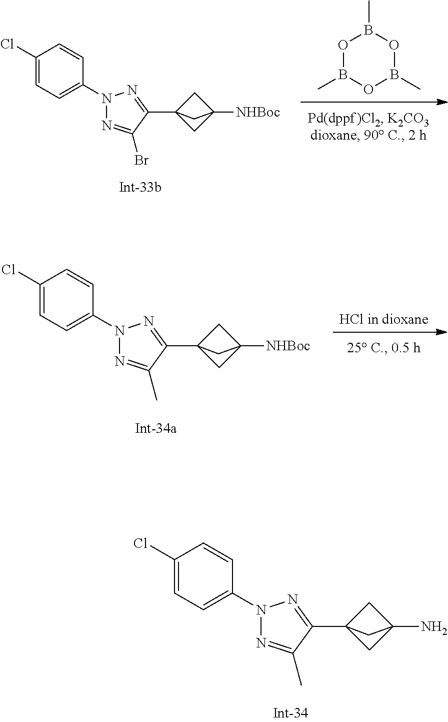

### Step 1: Preparation of tert-butyl N-[3-[2-(4-chlorophenyl)-5-methyl-triazol-4-yl]-1-bicyclo[1.1.1]pentanyl]carbamate (Int-34a)

To a mixture of tert-butyl N-[3-[5-bromo-2-(4-chlorophenyl)triazol-4-yl]-1-bicyclo[1.1.1]pentanyl]carbamate (Int-33b, 80 mg, 0.18 mmol) in dioxane (5 ml) was added 2,4,6-trimethyl-1,3,5,2,4,6-trioxatriborinane (113.4 mg, 0.9 mmol), Pd(dppf)Cl₂ (16.3 mg, 0.02 mmol) and K₂CO₃ (124.2 mg, 0.9 mmol). After being stirred at 90° C. for 2 h, the reaction was extracted with ethyl acetate (3×10 mL) and washed with water (10 mL) and brine (10 mL). The organic layer was dried over Na₂SO₄ and concentrated under reduced pressure. The residue was purified by flash column (eluting with EtOAc/PE=20/80) to afford tert-butyl N-[3-[2-(4-chlorophenyl)-5-methyl-triazol-4-yl]-1-bicyclo[1.1.1]pentanyl]carbamate (Int-34a, 40 mg, 59%) as colorless oil. MS obsd. (ESI⁺) [(M+H-56)⁺]: 375.1.

### Step 2: Preparation of 3-[2-(4-chlorophenyl)-5-methyl-triazol-4-yl]bicyclo[1.1.1]pentan-1-amine (Int-34)

tert-Butyl N-[3-[2-(4-chlorophenyl)-5-methyl-triazol-4-yl]-1-bicyclo[1.1.1]pentanyl]carbamate (Int-34a, 40 mg, 0.18 mmol) was placed in a round-bottomed flask, followed by the addition of a solution of HCl in 1,4-dioxane (4.0 M, 5 mL). After being stirred at rt for 0.5 h, the reaction mixture was concentrated under reduced pressure to give 3-[2-(4-chlorophenyl)-5-methyl-triazol-4-yl]bicyclo[1.1.1]pentan-1-amine (Int-34, 30 mg, HCl salt) as a white solid, which was used for the next step without further purification. MS obsd. (ESI⁺) [(M+H)⁺]: 275.1.

### Intermediate Int-35

### 3-[2-(p-tolyl)triazol-4-yl]bicyclo[1.1.1]pentan-1-amine

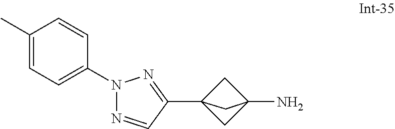 [SMILES: Cc1ccc(-n2ncc(C34CC(N)(C3)C4)n2)cc1]

The title compound was prepared according to the following scheme:

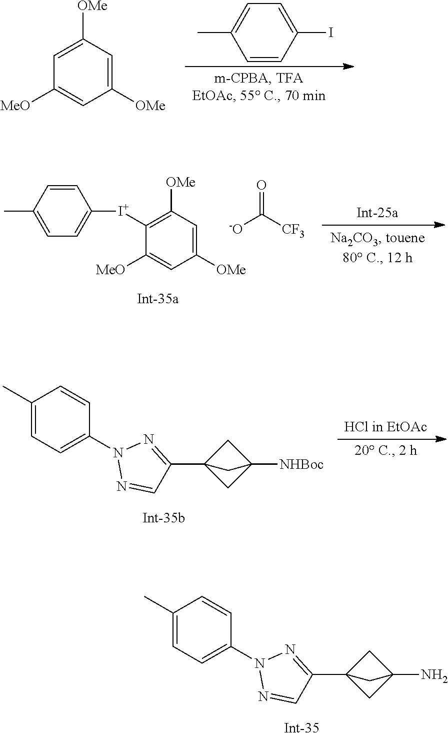

### Step 1: Preparation of p-tolyl(2,4,6-trimethoxyphenyl)iodonium 2,2,2-trifluoroacetate (Int-35a)

To a solution of 4-iodotoluene (1.0 g, 4.59 mmol) in EtOAc (5 mL) was added m-CPBA (1.12 g, 5.5 mmol) at 20° C., followed by the addition of trifluoroacetic acid (0.35 mL, 4.59 mmol) dropwise. After being stirred at 55° C. for 50 minutes, 1,3,5-trimethoxybenzene (0.77 g, 4.59 mmol) was added and the resulting mixture was stirred for another 20 minutes. The reaction mixture was concentrated in vacuum. The residue was dissolved in EtOAc (10 mL) and standed at 0° C. for 2 h. The product was crystallized and collected by filtration, dried under reduced pressure to afford p-tolyl-(2,4,6-trimethoxyphenyl)iodonium 2,2,2-trifluoroacetate (Int-35a, 950 mg, 41.6%) as a white solid. ¹H NMR (400 MHz, DMSO-d₆) δ ppm: 7.80 (d, J=8.4 Hz, 2H), 7.28-7.26 (d, J=8.0 Hz, 2H), 6.45 (s, 2H), 3.94 (s, 6H), 3.86 (s, 3H), 2.32 (s, 3H).

### Step 2: Preparation of tert-butyl (3-(2-(p-tolyl)-2H-1,2,3-triazol-4-yl)bicyclo[1.1.1]pentan-1-yl)carbamate (Int-35b)

To a solution of tert-butyl N-[3-(2H-triazol-4-yl)-1-bicyclo[1.1.1]pentanyl]carbamate (Int-25, 150.0 mg, 0.60 mmol) in toluene (3 mL) was added p-tolyl-(2,4,6-trimethoxyphenyl)iodonium 2,2,2-trifluoroacetate (Int-35a, 358.3 mg, 0.72 mmol) and Na₂CO₃ (76.22 mg, 0.72 mmol) at 20° C. After being stirred at 80° C. for 12 h, the mixture was poured into water (50 mL) and extracted with EtOAc (2×50 mL). The combined organic layer was washed with brine (100 mL) and dried over Na₂SO₄. After filtration and concentration, the residue was purified by silica gel column (eluting with PE/EtOAc=10/1 to 6/1) to afford tert-butyl N-[3-[2-(p-tolyl)triazol-4-yl]-1-bicyclo[1.1.1]pentanyl]carbamate (Int-35b, 95 mg, 46.6%) as a white solid. MS obsd. (ESI⁺) [(M+H)⁺]: 341.1. ¹H NMR (400 MHz, DMSO-d₆) δ ppm: 7.92 (s, 1H), 7.84-7.82 (m, 2H), 7.66 (s, 1H), 7.35-7.33 (m, 2H), 2.35 (s, 3H), 2.26 (s, 6H), 1.40 (s, 9H).

### Step 3: Preparation of 3-(2-(p-tolyl)-2H-1,2,3-triazol-4-yl)bicyclo[1.1.1]pentan-1-amine hydrochloride (Int-35)

To a solution of tert-butyl N-[3-[2-(p-tolyl)triazol-4-yl]-1-bicyclo[1.1.1]pentanyl]carbamate (Int-35b, 85.0 mg, 0.25 mmol) in a solution of HCl in EtOAc (4M, 2.13 mL) was stirred at 20° C. for 2 h. The reaction mixture was concentrated in vacuum to afford 3-[2-(p-tolyl)triazol-4-yl]bicyclo[1.1.1]pentan-1-amine (Int-35, 69.1 mg, HCl salt) as a white solid, which was used for the next step without further purification. MS obsd. (ESI⁺) [(M+H)⁺]: 241.1.

### Intermediate Int-36

### 3-[2-(4-methoxyphenyl)triazol-4-yl]bicyclo[1.1.1]pentan-1-amine

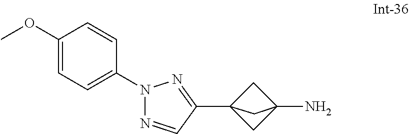 [SMILES: COc1ccc(-n2ncc(C34CC(N)(C3)C4)n2)cc1]

The title compound was prepared in analogy to the procedure described for the preparation of 3-[2-(p-tolyl)triazol-4-yl]bicyclo[1.1.1]pentan-1-amine (Int-35), by using 4-iodoanisole instead of 4-iodotoluene. MS obsd. (ESI⁺) [(M+H)⁺]: 257.1

### Intermediate Int-37

### 5-(1-methylsulfonylcyclopropyl)furan-2-carboxylic acid

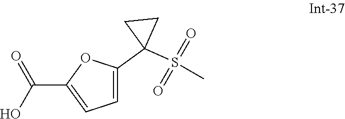

The title compound was prepared according to the following scheme:

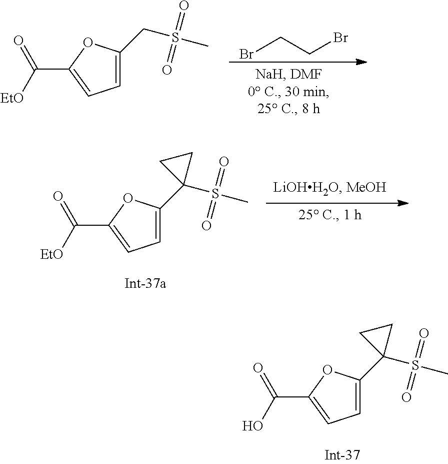

### Step 1: Preparation of ethyl 5-(1-methylsulfonylcyclopropyl)furan-2-carboxylate (Int-37a)

To a solution of ethyl 5-(methylsulfonylmethyl)furan-2-carboxylate (500 mg, 2.15 mmol) in anhydrous DMF (15 mL) was added sodium hydride (155 mg, 6.46 mmol, 60% in oil) at 0° C. After being stirred at 0° C. for 30 min, 1,2-dibromoethane (0.19 mL, 2.15 mmol) was added. The reaction was stirred at 25° C. for 8 h and then quenched with water (1 mL). The resulting solution was diluted with EtOAc (100 mL) and washed with water (4×50 mL) and brine (100 mL). The organic layer was dried over Na₂SO₄ and concentrated in vacuum. The residue was purified by silica gel column (eluting with PE/EtOAc=1/1) to give ethyl 5-(1-methylsulfonylcyclopropyl)furan-2-carboxylate (Int-37a, 200 mg) as light yellow oil. MS obsd. (ESI⁺) [(M+H)⁺]: 259.0.

### Step 2: Preparation of 5-(1-methylsulfonylcyclopropyl)furan-2-carboxylic acid (Int-37)

To a solution of ethyl 5-(1-methylsulfonylcyclopropyl)furan-2-carboxylate (Int-37a, 100 mg, 0.39 mmol) in methanol (5 mL) was added LiOH H₂O (17 mg, 0.43 mmol). The reaction mixture was stirred at 25° C. for 1 h and then concentrated in vacuum to give 5-(1-methylsulfonylcyclopropyl)furan-2-carboxylic acid (Int-37, 73 mg, lithium salt) as a light yellow solid. The crude was used for next step without further purification. MS obsd. (ESI⁺) [(M+H)⁺]: 231.1.

### Intermediate Int-38

### 3-(methylsulfonylmethyl)-1,2,4-thiadiazole-5-carboxylic acid

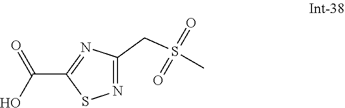

The title compound was prepared according to the following scheme:

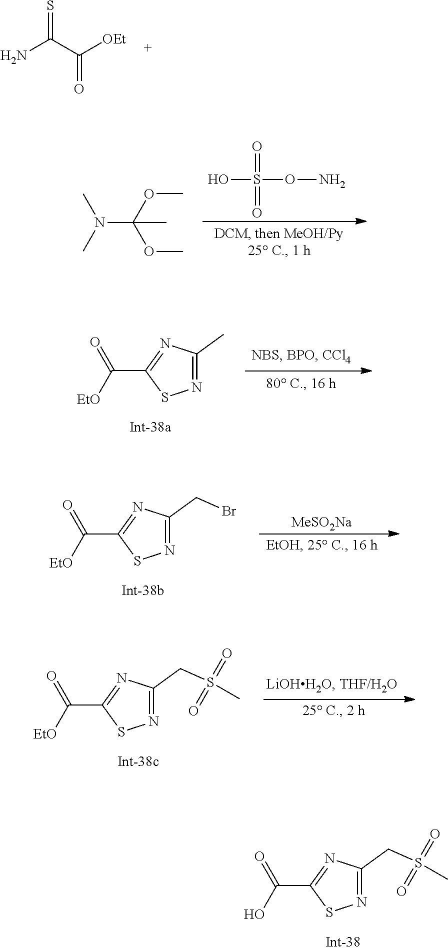

### Step 1: Preparation of ethyl 3-methyl-1,2,4-thiadiazole-5-carboxylate (Int-38a)

To a solution of N,N-dimethylacetamide dimethyl acetal (11.0 g, 82.6 mmol) in DCM (10 mL) was added ethyl thiooxamate (10.0 g, 75.1 mmol). After being stirred at 25° C. for 10 min, DCM was removed in vacuo. The residue was dissolved in methanol (10 mL), to which pyridine (12.2 mL, 150.2 mmol) and a solution of hydroxylamine-O-sulfonic acid (9.34 g, 82.6 mmol) in methanol (10 mL) were added at 0° C. slowly. The reaction mixture was warmed to ambient temperature and stirred for another 1 h. The resulting solution was concentrated in vacuum to remove methanol and the residue was dissolved in EtOAc (100 mL). The resulting solution was washed with water (2×40 mL), brine (40 mL) and dried over Na₂SO₄. The solution was concentrated and the residue was purified by silica gel column (eluting with PE/EtOAc=4/1) to afford ethyl 3-methyl-1,2,4-thiadiazole-5-carboxylate (Int-38a, 8.18 g) as orange oil. MS obsd. (ESI⁺) [(M+H)⁺]: 173.1.

### Step 2: Preparation of ethyl 3-(bromomethyl)-1,2,4-thiadiazole-5-carboxylate (Int-38b)

To a solution of ethyl 3-methyl-1,2,4-thiadiazole-5-carboxylate (Int-38a, 10.0 g, 58.1 mmol) in CCl₄ (82 mL) were added NBS (12.0 g, 69.7 mmol) and BPO (21.1 g, 87.1 mmol) at 25° C. After being stirred at 80° C. for 16 h, the solution was concentrated to remove CCl₄. The residue was redissolved in EtOAc (100 mL) and washed with water (2×40 mL) and brine (40 mL). The organic layer was dried over Na₂SO₄ and concentrated in vacuum. The residue was purified by silca gel column (eluting with PE/EtOAc=4/1) to afford ethyl 3-(bromomethyl)-1,2,4-thiadiazole-5-carboxylate (Int-38b, 7.26 g) as orange oil. MS obsd. (ESI⁺) [(M+H)⁺]: 250.9.

### Step 3: Preparation of ethyl 3-(methylsulfonylmethyl)-1,2,4-thiadiazole-5-carboxylate (Int-38c)

To a solution of ethyl 3-(bromomethyl)-1,2,4-thiadiazole-5-carboxylate (Int-38b, 7.69 g, 30.6 mmol) in ethanol (120 mL) was added sodium methanesulfinate (4.69 g, 45.9 mmol) at 25° C. After being stirred for 16 h, the resulting solution was concentrated in vacuum. The residue was dissolved in EtOAc (100 mL), washed with water (2×40 mL) and brine (40 mL). The organic layer was dried over Na₂SO₄ and concentrated in vacuum. The residue was purified by silica gel column (eluting with PE/EtOAc=2/3) to afford ethyl 3-(methylsulfonylmethyl)-1,2,4-thiadiazole-5-carboxylate (Int-38c, 6.12 g) as a white solid. MS obsd. (ESI⁺) [(M+H)⁺]: 251.1.

### Step 4: Preparation of 3-(methylsulfonylmethyl)-1,2,4-thiadiazole-5-carboxylic acid (Int-38)

To a solution of ethyl 3-(methylsulfonylmethyl)-1,2,4-thiadiazole-5-carboxylate (Int-38c, 62.0 mg, 0.25 mmol) in THF (2 mL) and water (1 mL) was added lithium hydroxide (8.9 mg, 0.37 mmol).

After being stirred at 25° C. for 2 h, water (30 ml) was added and the acidified mixture was extracted with EtOAc (3×20 mL). The combined organic layer was dried over Na₂SO₄ and concentrated. The residue was purified by flash column chromatography (eluting with DCM/MeOH=95/5) to afford 3-(methylsulfonylmethyl)-1,2,4-thiadiazole-5-carboxylic acid (Int-38, 51 mg, 74.12%) as a white solid. MS obsd. (ESI⁺) [(M+H)⁺]: 223.1.

### Intermediate Int-39

### 3-(1-methylsulfonylcyclopropyl)-1,2,4-thiadiazole-5-carboxylic acid

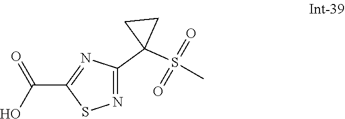

The title compound was prepared according to the following scheme:

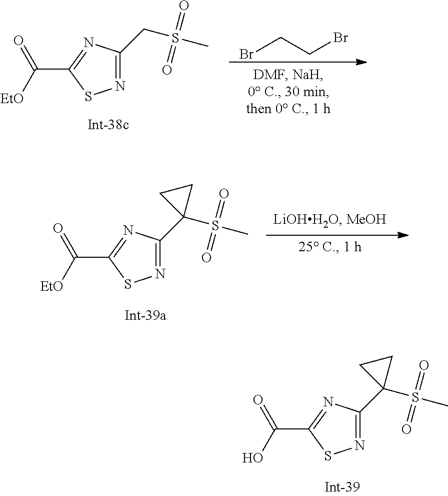

### Step 1: Preparation of ethyl 3-(1-methylsulfonylcyclopropyl)-1,2,4-thiadiazole-5-carboxylate (Int-39a)

To a solution of ethyl 3-(methylsulfonylmethyl)-1,2,4-thiadiazole-5-carboxylate (Int-38c, 500 mg, 2.0 mmol) in DMF (10 mL) was added sodium hydride in several portions (120 mg, 4.99 mmol, 60% in oil) under nitrogen atmosphere at 0° C. After being stirred at 0° C. for 30 min, ethylene dibromide (563 mg, 3.00 mmol) was added dropwise. The reaction mixture was stirred at 0° C. for 1 h. The resulting solution was diluted with EtOAc (100 mL) at 0° C. and then ice-water (50 mL). The organic layer was washed with brine (50 mL), dried over Na₂SO₄ and concentrated in vacuum. The residue was purified by silica gel column (eluting with PE/EtOAc=7/3) to afford ethyl 3-(1-methylsulfonylcyclopropyl)-1,2,4-thiadiazole-5-carboxylate (Int-39a, 298 mg) as a white solid. MS obsd. (ESI⁺) [(M+H)⁺]: 277.0. ¹H NMR (400 MHz, CDCl₃) δ ppm: 4.52 (q, J=7.2 Hz, 2H), 3.37 (s, 3H), 1.98-2.04 (m, 2H), 1.81-1.87 (m, 2H), 1.45 (t, J=7.2 Hz, 3H).

### Step 2: Preparation of 3-(1-methylsulfonylcyclopropyl)-1,2,4-thiadiazole-5-carboxylic acid (Int-39)

To a solution of ethyl 3-(1-methylsulfonylcyclopropyl)-1,2,4-thiadiazole-5-carboxylate (Int-39a, 298 mg, 1.1 mmol) in MeOH (10 mL) was added LiOH H₂O (88 mg, 1.1 mmol) and 1 drop of water. The reaction was stirred at 25° C. for 2 h. The resulting solution was concentrated in vacuum to give 3-(1-methylsulfonylcyclopropyl)-1,2,4-thiadiazole-5-carboxylic acid (Int-39, 279 mg, lithium salt) as a white solid, which was used for next step without further purification. MS obsd. (ESI⁺) [(M+H)⁺]: 249.1.

### Intermediate Int-40

### 3-(1-methylsulfonylcyclopropyl)-1H-pyrazole

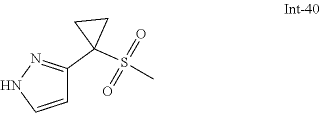

The title compound was prepared according to the following scheme:

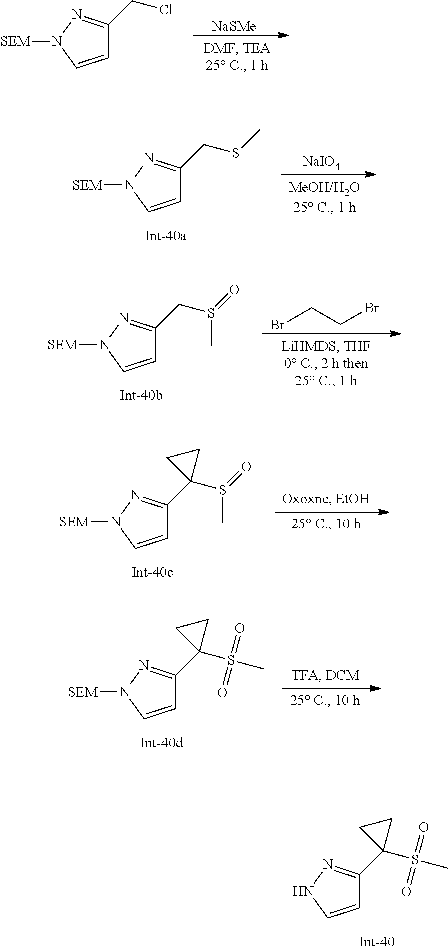

### Step 1: Preparation of trimethyl-[2-[[3-(methylsulfanylmethyl)pyrazol-1-yl]methoxy]ethyl]silane (Int-40a)

To a solution of 2-[[3-(chloromethyl)pyrazol-1-yl]methoxy]ethyl-trimethyl-silane (42.0 g, 170.17 mmol) in DMF (200 mL) was added methanethiol sodium salt (14.31 g, 204.21 mmol) and triethylamine (71.16 mL, 510.51 mmol) at 25° C. After being stirred at 25° C. for 1 h, the mixture was purified by HPLC to give trimethyl-[2-[[3-(methylsulfanylmethyl)pyrazol-1-yl]methoxy]ethyl]silane (Int-40a, 31.56 g, 71.74%) as a brown solid. MS obsd. (ESI⁺) [(M+H)⁺]: 259.1.

### Step 2: Preparation of trimethyl-[2-[[3-(methylsulfinylmethyl)pyrazol-1-yl]methoxy]ethyl]silane (Int-40b)

To a mixture of trimethyl-[2-[[3-(methylsulfanylmethyl)pyrazol-1-yl]methoxy]ethyl]silane (Int-40a, 6 g, 23.21 mmol) in methanol (100 mL) and water (20 mL) was added sodium periodate (24.82 g, 116.05 mmol) at 25° C. After being stirred at 25° C. for 1 h, the reaction was quenched with aqueous solution of Na₂SO₃ and NaHCO₃, extracted with EtOAc (3×500 mL). The organic layers were combined, washed with brine (600 mL) and dried over Na₂SO₄. The organic layer was filtered and concentrated to give trimethyl-[2-[[3-(methylsulfinylmethyl)pyrazol-1-yl]methoxy]ethyl]silane (Int-40b, 6 g, 94.26%) as yellow oil. MS obsd. (ESI⁺) [(M+H)⁺]: 275.2.

### Step 3: Preparation of 3-(1-(methylsulfinyl)cyclopropyl)-1-((2-(trimethylsilyl)ethoxy)methyl)-1H-pyrazole (Int-40c)

To a solution of trimethyl-[2-[[3-(methylsulfinylmethyl)pyrazol-1-yl]methoxy]ethyl]silane (Int-40b, 4 g, 14.54 mmol) in THF (20 mL) was added LiHMDS (58.2 mL, 58.16 mmol) at 0° C. under N₂. After being stirred for another 1 h, 1,2-dibromoethane (13.66 g, 72.7 mmol) was added. The mixture was stirred at 0° C. for 1 h followed by stirring at 20° C. for another 1 h. After being quenched with aqueous NH₄Cl (300 mL), the resulting mixture was extracted with EtOAc (3×100 mL). The combined organic layer was dried over Na₂SO₄ and concentrated. The residue was purified by flash column chromatography (eluting with 30%˜90% of PE in EtOAc) to give 3-(1-(methylsulfinyl)cyclopropyl)-1-((2-(trimethylsilyl)ethoxy)methyl)-1H-pyrazole (Int-40c, 1.5 g, 34.25%) as yellow oil. MS obsd. (ESI⁺) [(M+H)⁺]: 301.1.

### Step 4: Preparation of trimethyl-[2-[[3-(1-methylsulfonylcyclopropyl)pyrazol-1-yl]methoxy]ethyl]silane (Int-40d)

To a solution of trimethyl-[2-[[3-(1-methylsulfinylcyclopropyl)pyrazol-1-yl]methoxy]ethyl]silane (Int-40c, 1.5 g, 4.99 mmol) in ethanol (20 mL) was added Oxone (3.99 g, 6.49 mmol). After being stirred at 25° C. for 10 h, the mixture was concentrated and redissolved in water (50 ml), extracted with EtOAc (2×30 mL) and dried with Na₂SO₄. The solvent was removed in vacuo to give the product trimethyl-[2-[[3-(1-methylsulfonylcyclopropyl)pyrazol-1-yl]methoxy]ethyl]silane (Int-40d, 1.3 g, 65.83%) as yellow oil. MS obsd. (ESI⁺) [(M+H)⁺]: 317.1.

### Step 5: Preparation of 3-(1-methylsulfonylcyclopropyl)-1H-pyrazole (Int-40)

To a solution of trimethyl-[2-[[3-(1-methylsulfonylcyclopropyl)pyrazol-1-yl]methoxy]ethyl]silane (Int-40d, 1.3 g, 4.11 mmol) in DCM (5 mL) was added TFA (5.0 mL, 4.11 mmol). After being stirred at 25° C. for 10 h, the mixture was concentrated and washed with aqueous NaHCO₃ (100 mL), followed by extraction with EtOAc (3×50 mL). The combined organic layer was dried over Na₂SO₄ and concentrated in vacuo. The residue was purified by HPLC (eluting with 0 to 10% of ACN in water) to give 3-(1-methylsulfonylcyclopropyl)-1H-pyrazole (Int-40, 900 mg, 117.65%) as yellow oil. MS obsd. (ESI⁺) [(M+H)⁺]: 187.1.

### Intermediate Int-41

### 2-(methylsulfonylmethyl)oxazole-5-carboxylic acid

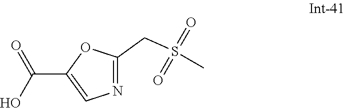

The title compound was prepared according to the following scheme:

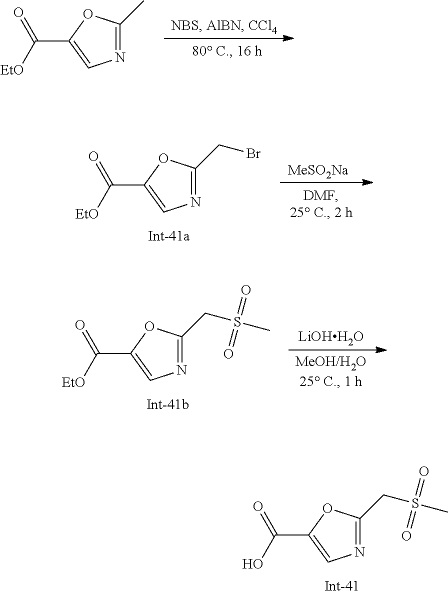

### Step 1: Preparation of ethyl 2-(bromomethyl)oxazole-5-carboxylate (Int-41)

A mixture of ethyl 2-methyloxazole-5-carboxylate (2.5 g, 16.1 mmol), NBS (4.3 g, 24.2 mmol) and AIBN (1.06 g, 6.45 mmol) in CCl₄ (50 mL) was stirred at 80° C. for 16 h. The reaction mixture was filtered and the filtrate was concentrated in vacuum. The residue was purified by silica gel column (eluting with PE/EtOAc=9/1) to afford ethyl 2-(bromomethyl)oxazole-5-carboxylate (Int-41a, 3.77 g) as a light yellow solid. MS obsd. (ESI⁺) [(M+H)⁺]: 234.0.

### Step 2: Preparation of ethyl 2-(methylsulfonylmethyl)oxazole-5-carboxylate (Int-41b)

A mixture of ethyl 2-(bromomethyl)oxazole-5-carboxylate (Int-41a, 3.77 g, 16.1 mmol) and sodium methanesulfinate (2.47 g, 24.2 mmol) in DMF (15 mL) was stirred at 25° C. for 2 h. The mixture was concentrated in vacuum and the residue was purified by silica gel column (eluting with PE/EtOAc=7/3) to afford ethyl 2-(methylsulfonylmethyl)oxazole-5-carboxylate (Int-41b, 600 mg) as a white solid. MS obsd. (ESI⁺) [(M+H)⁺]: 234.1.

### Step 3: Preparation of [2-(methylsulfonylmethyl)oxazole-5-carbonyl]oxylithium (Int-41)

A mixture of ethyl 2-(methylsulfonylmethyl)oxazole-5-carboxylate (50 mg, 0.21 mmol) and LiOH H₂O (18 mg, 0.43 mmol) in a mixture of methanol (5 mL) and water (0.1 mL) was stirred at 25° C. for 2 h. After that, the mixture was concentrated in vacuum to afford 2-(methylsulfonylmethyl)oxazole-5-carboxylic acid (Int-41, 44 mg, lithium salt) as a light red solid. The crude was used for next step without further purification. MS obsd. (ESI⁺) [(M+H)⁺]: 206.0.

### Intermediate Int-42

### 2-(methylsulfonylmethyl)oxazole-5-carboxylic acid

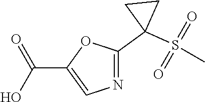

The title compound was prepared according to the following scheme:

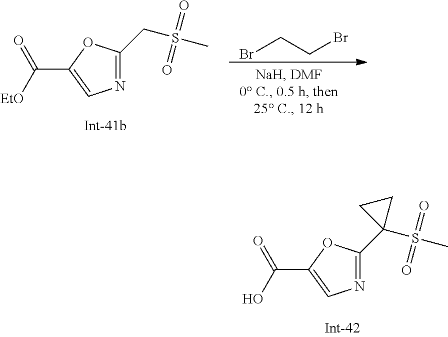

To a solution of ethyl 2-(methylsulfonylmethyl)oxazole-5-carboxylate (Int-41b, 50.0 mg, 0.21 mmol) in DMF (1 mL) was added NaH (12.86 mg, 0.54 mmol) at 0° C. After being stirred for 0.5 h, 1,2-dibromoethane (60.41 mg, 0.32 mmol) was added and the mixture was stirred at 25° C. for 12 h. The solution of 2-(1-methylsulfonylcyclopropyl)oxazole-5-carboxylic acid (Int-42, 20 mg, sodium salt, 40.35%) in DMF was used directly in next step. MS obsd. (ESI⁺): 232.0 [(M+H)⁺].

### Intermediate Int-43

### 5-(1-methylsulfonylcyclopropyl)-1,3,4-oxadiazole-2-carboxylic acid

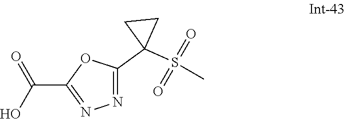

The title compound was prepared according to the following scheme:

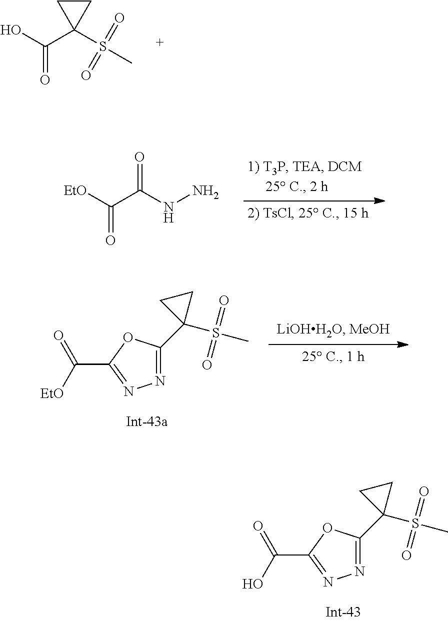

### Step 1: Preparation of ethyl 5-(1-methylsulfonylcyclopropyl)-1,3,4-oxadiazole-2-carboxylate (Int-43a)

To a solution of 1-methylsulfonylcyclopropanecarboxylic acid (3.00 g, 18.3 mmol), ethyl 2-hydrazino-2-oxo-acetate (2.41 g, 18.3 mmol) in DCM (100 mL) were added TEA (5.55 g, 54.8 mmol) and T₃P (11.6 g, 36.6 mmol). After being stirred at room temperature for 2 h, TsCl (10.5 g, 54.8 mmol) was added at 0° C. The reaction mixture was warmed to room temperature and stirred for further 15 h. The resulting solution was concentrated in vacuum. The residue was purified by silica gel column (eluting with PE/EtOAc=3/2) to afford ethyl 5-(1-methylsulfonylcyclopropyl)-1,3,4-oxadiazole-2-carboxylate (Int-43a, 2.1 g) as yellow oil. MS obsd. (ESI⁺) [(M+H)⁺]: 261.1. ¹H NMR (400 MHz, CDCl₃) δ ppm: 4.50 (q, J=7.2 Hz, 2H), 3.25 (s, 3H), 2.02-2.06 (m, 2H), 1.73-1.79 (m, 2H), 1.43 (t, J=7.2 Hz, 3H).

### Step 2: Preparation of 5-(1-methylsulfonylcyclopropyl)-1,3,4-oxadiazole-2-carboxylic acid (Int-43)

A solution of ethyl 5-(1-methylsulfonylcyclopropyl)-1,3,4-oxadiazole-2-carboxylate (Int-43a, 350 mg, 1.34 mmol) and LiOH H₂O (59 mg, 1.41 mmol) in methanol (10 mL) was stirred at 25° C. for 1 h. The reaction mixture was concentrated in vacuum to afford 5-(1-methylsulfonylcyclopropyl)-1,3,4-oxadiazole-2-carboxylic acid (Int-43, 300 mg, lithium salt) as a yellow solid. The crude was used for next step without further purification. MS obsd. (ESI⁺) [(M+H)⁺]: 233.1.

### Intermediate Int-44

### 3-(1-methyl-1-methylsulfonyl-ethyl)benzoic acid

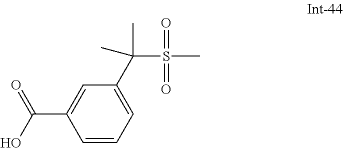 [SMILES: CC(C)(c1cccc(C(=O)O)c1)S(C)(=O)=O]

The title compound was prepared according to the following scheme:

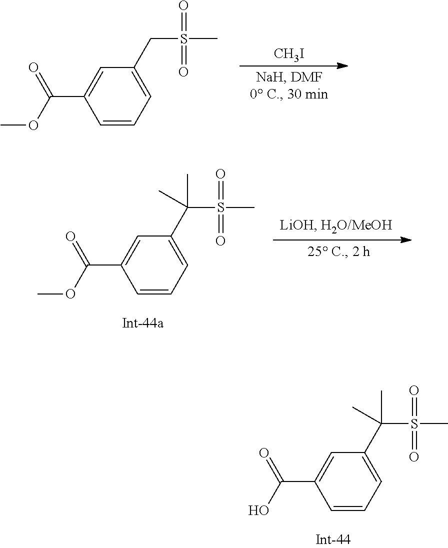

### Step 1: Preparation of methyl 3-(1-methyl-1-methylsulfonyl-ethyl) benzoate (Int-44a)

To a solution of methyl 3-(methylsulfonylmethyl)benzoate (400.0 mg, 1.75 mmol) in DMF (10 mL) was added sodium hydride (88.32 mg, 3.68 mmol) at 0° C. under nitrogen atmosphere. After being stirred at 0° C. for 30 min, iodomethane (572.08 mg, 4.03 mmol) was added into above mixture at 0° C. under nitrogen atmosphere, followed by stirring for another 30 min. The resulting mixture was dissolved in ethyl acetate and washed with water (3×150 mL). The combined organic phase was concentrated under reduced pressure, the residue was further purified by silica gel column to give methyl 3-(1-methyl-1-methylsulfonyl-ethyl) benzoate (Int-44a, 436 mg, 97.07%) as a light yellow solid. MS obsd. (ESI⁺) [(M+H)⁺]: 279.1.

### Step 2: Preparation of 3-(1-methyl-1-methylsulfonyl-ethyl)benzoic acid (Int-44)

To a solution of methyl 3-(1-methyl-1-methylsulfonyl-ethyl) benzoate (Int-44a, 436.0 mg, 1.7 mmol) in methanol (7.4 mL) was added aqueous lithium hydroxide (0.02 mL, 1.7 mmol) at ambient temperature. After being stirred for 2 h, the resulting solution was concentrated under reduced pressure to give 3-(1-methyl-1-methylsulfonyl-ethyl)benzoic acid (Int-44a, 412 mg, lithium salt) as a light yellow solid, which was used directly without further purification. MS obsd. (ESI⁺) [(M+H)⁺]: 265.1.

### Intermediate Int-45

### 6-(1-methylsulfonylcyclopropyl)pyridine-2-carboxylic acid

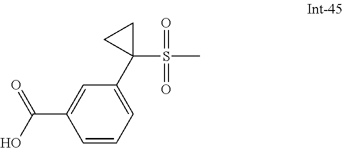 [SMILES: CS(=O)(=O)C1(c2cccc(C(=O)O)c2)CC1]

The title compound was prepared according to the following scheme:

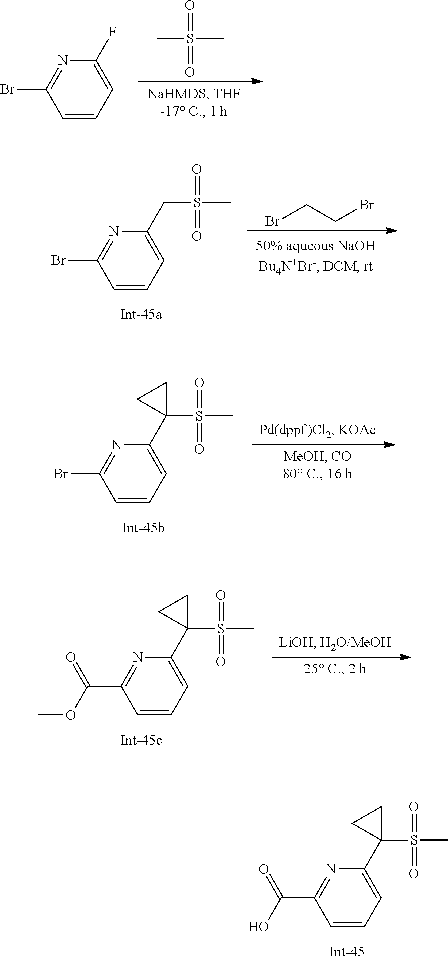

### Step 1: Preparation of 2-bromo-6-(methylsulfonylmethyl)pyridine (Int-45a)

To a solution of sodium bis(trimethylsilyl)amide (5.21 g, 28.41 mmol) in THF (20 mL) was added 2-bromo-6-fluoropyridine (1.0 g, 5.68 mmol) at −17° C. under nitrogen atmosphere. After being stirred at −17° C. for 30 min, dimethyl sulfone (1.07 g, 11.36 mmol) was added and the mixture was stirred for further 1 h. The reaction was quenched by the addition of saturated NaCl solution and extracted with ethyl acetate. The organic phase was dried over Na₂SO₄, filtered and concentrated under reduced pressure to remove the solvent to give 2-bromo-6-(methylsulfonylmethyl)pyridine (Int-45a, 1.31 g, 92.18%) as a brown solid. MS obsd. (ESI⁺) [(M+H)⁺]: 250.0.

### Step 2: Preparation of 2-bromo-6-(1-methylsulfonylcyclopropyl)pyridine (Int-45b)

To a solution of 2-bromo-6-(methylsulfonylmethyl)pyridine (Int-45a, 1.39 g, 5.56 mmol) in DCM (20.65 mL) was added 1,2-dibromoethane (5.22 g, 27.81 mmol), NaOH (11.7 g, 50% aqueous, 292.6 mmol) and tetrabutylammonium bromide (1.48 g, 6.12 mmol) at ambient temperature. After being stirred for 5 h under nitrogen atmosphere, the resulting mixture was concentrated under reduced pressure to remove the solvent. The residue was dissolved in DCM (30 mL) and extracted with H₂O (3×50 mL). The combined organic phase was concentrated under reduced pressure. The residue was further purified by silica gel column (eluting with PE to PE:EA=3:1) to give 2-bromo-6-(1-methylsulfonylcyclopropyl)pyridine (Int-45b, 445 mg, 28.97%) as a brown solid. MS obsd. (ESI⁺) [(M+H)⁺]: 276.0.

### Step 3: Preparation of methyl 6-(1-methylsulfonylcyclopropyl) pyridine-2-carboxylate (Int-45c)

To a solution of 2-bromo-6-(1-methylsulfonylcyclopropyl)pyridine (Int-45b, 210.0 mg, 0.76 mmol) in methanol (10 mL) was added potassium acetate (223.89 mg, 2.28 mmol) and [1,1′-bis(diphenylphosphino)ferrocene]dichloropalladium (II) (55.64 mg, 0.08 mmol). After being heated with stirring at 80° C. for 16 h under CO atmosphere, the resulting mixture was concentrated to remove solvent under reduced pressure. The residue was dissolved in ethyl acetate (20 mL) and washed with water (3×30 mL). The combined organic phase was concentrated under reduced pressure and further purified by silica gel column (eluting with PE to PE:EA=1:1) to give methyl 6-(1-methylsulfonylcyclopropyl)pyridine-2-carboxylate (Int-45c, 111 mg, 57.18%) as a light yellow solid. MS obsd. (ESI⁺) [(M+H)⁺]: 256.1.

### Step 4: Preparation of 6-(1-methylsulfonylcyclopropyl)pyridine-2-carboxylic acid (Int-45)

To a solution of methyl 6-(1-methylsulfonylcyclopropyl)pyridine-2-carboxylate (Int-45c, 111 mg, 0.43 mmol) in methanol (10 mL) and a drop of water was added lithium hydroxide (10.3 mg, 0.43 mmol) at ambient temperature. After being stirred at ambient temperature for 2 h, the resulting solution was concentrated to remove the methanol under reduced pressure to give 6-(1-methylsulfonylcyclopropyl)pyridine-2-carboxylic acid (Int-45, 101 mg, lithium salt) as a light yellow solid. MS obsd. (ESI⁺) [(M+H)⁺]: 242.0.

### Intermediate Int-46

### 2-(1-methylsulfonylcyclopropyl)pyridine-4-carboxylic acid

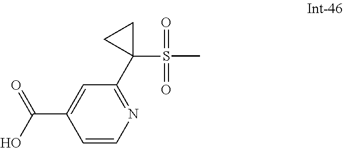 [SMILES: CS(=O)(=O)C1(c2cc(C(=O)O)ccn2)CC1]

The title compound was prepared in analogy to the procedure described for the preparation of 6-(1-methylsulfonylcyclopropyl)pyridine-2-carboxylic acid (Int-45), by using 4-bromo-2-fluoropyridine instead of 2-bromo-6-fluoropyridine. MS obsd. (ESI⁺) [(M+H)⁺]: 242.0.

### Example 1

### N-[3-[4-(4-chlorophenyl)thiazol-2-yl]-1-bicyclo[1.1.1]pentanyl]-5-(1-methylsulfonylcyclopropyl)furan-2-carboxamide

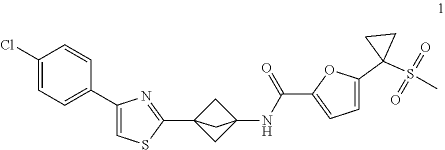 [SMILES: CS(=O)(=O)C1(c2ccc(C(=O)NC34CC(c5nc(-c6ccc(Cl)cc6)cs5)(C3)C4)o2)CC1]

The title compound was prepared according to the following scheme:

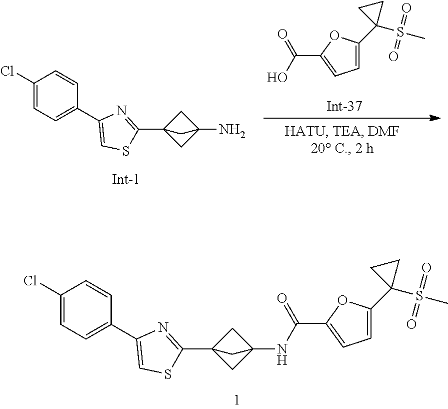 [SMILES: NC12CC(c3nc(-c4ccc(Cl)cc4)cs3)(C1)C2 ; CS(=O)(=O)C1(c2ccc(C(=O)O)o2)CC1 ; CS(=O)(=O)C1(c2ccc(C(=O)NC34CC(c5nc(-c6ccc(Cl)cc6)cs5)(C3)C4)o2)CC1]

To a solution of 5-(1-methylsulfonylcyclopropyl)furan-2-carboxylic acid (Int-37, 166.37 mg, 0.72 mmol) in DMF (5 mL) were added O-(7-azabenzotriazol-1-yl)-N,N,N′,N′-tetramethyluronium hexafluorophosphate (HATU, 329.7 mg, 0.87 mmol), triethylamine (0.3 mL, 2.17 mmol) and 3-[4-(4-chlorophenyl)thiazol-2-yl]bicyclo[1.1.1]pentan-1-amine (Int-1, HCl salt, 200.0 mg). After being stirred at 20° C. for 2 h, the resulting solution was diluted with water (30 mL) and extracted with ethyl acetate (3×20 mL). The combined organic layer was dried over Na₂SO₄ and concentrated under reduced pressure. The residue was purified by flash column (eluting with EtOAc/PE=40/60) to give N-[3-[4-(4-chlorophenyl)thiazol-2-yl]-1-bicyclo[1.1.1]pentanyl]-5-(1-methylsulfonylcyclopropyl)furan-2-carboxamide (Example 1, 117 mg, 33.11%) as a white solid. MS obsd. (ESI⁺) [(M+H)⁺]: 489.1. ¹H NMR (400 MHz, CD₃OD) δ ppm: 7.84-7.94 (m, 2H), 7.74 (s, 1H), 7.42 (d, J=8.8 Hz, 2H), 7.12 (d, J=3.6 Hz, 1H), 6.78 (d, J=3.6 Hz, 1H), 3.02 (s, 3H), 2.62 (s, 6H), 1.75-1.84 (m, 2H), 1.52-1.59 (m, 2H).

The following Example 2 to Example 52 were prepared in analogy to the procedure described for the preparation of Example 1, replacing Int-1 with corresponding “Amine” as indicated in Table 2, and replacing 5-(1-methylsulfonylcyclopropyl)furan-2-carboxylic acid with corresponding “Acid” as indicated in Table 2.

TABLE 2
Compounds synthesis and characterization

| Examples | Compound Name, Structure, MS (ESI⁺) and ¹H NMR | Amine & Acid |
|---|---|---|
| 2 | N-[3-[4-(3-chlorophenyl)thiazol-2-yl]-1- | Amine: Int-2 |
| | bicyclo[1.1.1]pentanyl]-5-(1-methylsulfonylcyclopropyl)furan- | Acid: Int-37 |
| | 2-carboxamide | |
| | 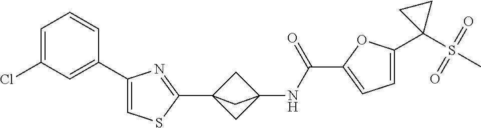 [SMILES: CS(=O)(=O)C1(c2ccc(C(=O)NC34CC(c5nc(-c6cccc(Cl)c6)cs5)(C3)C4)o2)CC1] | |
| | MS obsd. (ESI⁺) [(M + H)⁺]: 489.0. ¹H NMR (400 MHz, | |
| | CD₃OD) δ ppm: 7.95 (t, J = 7.6 Hz, 1H), 7.83 (d, J = 7.6 Hz, | |
| | 1H), 7.81 (s, 1H), 7.40 (d, J = 7.6 Hz, 1H), 7.35 (s, 1H), 7.12 | |
| | (d, J = 3.6 Hz, 1H), 6.78 (d, J = 3.6 Hz, 1H), 3.02 (s, 3H), 2.63 | |
| | (s, 6H), 1.74-1.82 (m, 2H), 1.52-1.59 (m, 2H). | |
| 3 | N-[3-[4-(2-chlorophenyl)thiazol-2-yl]-1- | Amine: Int-3 |
| | bicyclo[1.1.1]pentanyl]-5-(1-methylsulfonylcyclopropyl)furan- | Acid: Int-37 |
| | 2-carboxamide | |
| | 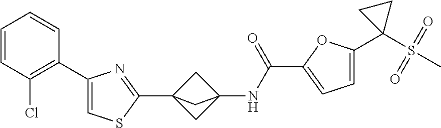 [SMILES: CS(=O)(=O)C1(c2ccc(C(=O)NC34CC(c5nc(-c6ccccc6Cl)cs5)(C3)C4)o2)CC1] | |
| | MS obsd. (ESI⁺) [(M + H)⁺]: 489.1. ¹H NMR (400 MHz, | |
| | CD₃OD) δ ppm: 7.68-7.81 (m, 2H), 7.44-7.60 (s, 1H), 7.35-3.40 | |
| | (m, 2H), 7.12 (d, J = 3.6 Hz, 1H), 6.78 (d, J = 3.6 Hz, 1H), 3.02 | |
| | (s, 3H), 2.62 (s, 6H), 1.75-1.83 (m, 2H), 1.51-1.59 (m, 2H). | |
| 4 | N-[3-[4-(3-chlorophenyl)thiazol-2-yl]-1- | Amine: Int-2 |
| | thiadiazole-5-carboxamide | Acid: Int-39 |
| | 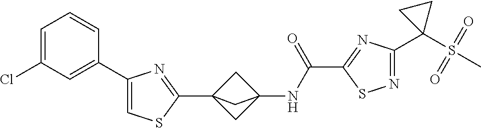 [SMILES: CS(=O)(=O)C1(c2nsc(C(=O)NC34CC(c5nc(-c6cccc(Cl)c6)cs5)(C3)C4)n2)CC1] | |
| | MS obsd. (ESI⁺) [(M + H)⁺]: 507.1. ¹H NMR (400 MHz, CDCl₃) | |
| | δ ppm: 7.91 (s, 1H), 7.76 (d, J = 7.6 Hz, 1H), 7.60 (s, 1H), 7.42 | |
| | (s, 1H), 7.28-7.38 (m, 2H), 3.30 (s, 3H), 2.71 (s, 6H), 2.01-2.05 | |
| | (m, 2H), 1.74-1.82 (m, 2H). | |
| 5 | N-[3-[4-(4-chlorophenyl)thiazol-2-yl]-1- | Amine: Int-1 |
| | bicyclo[1.1.1]pentanyl]-2-(methylsulfonylmethyl)oxazole-5- | Acid: Int-41 |
| | carboxamide | |
| | 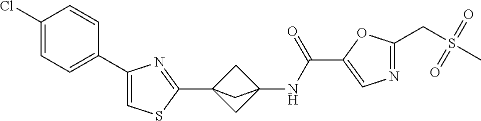 [SMILES: CS(=O)(=O)Cc1ncc(C(=O)NC23CC(c4nc(-c5ccc(Cl)cc5)cs4)(C2)C3)o1] | |
| | MS obsd. (ESI⁺) [(M + H)⁺]: 464.0. ¹H NMR (400 MHz, CDCl₃) | |
| | δ ppm: 8.28 (s, 1H), 7.83 (d, J = 8.6 Hz, 2H), 7.39 (s, 1H), 7.37 | |
| | (d, J = 8.6 Hz, 2H), 7.31 (s, 1H), 4.49 (s, 2H), 3.10 (s, 3H), 2.66 | |
| | (s, 6H). | |
| 6 | N-[3-[4-(4-chlorophenyl)thiazol-2-yl]-1- | Amine: Int-1 |
| | bicyclo[1.1.1]pentanyl]-2-(1- | Acid: Int-42 |
| | methylsulfonylcyclopropyl)oxazole-5-carboxamide | |
| | 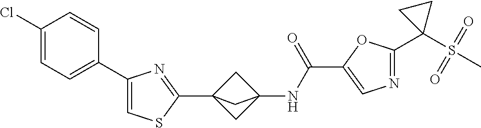 [SMILES: CS(=O)(=O)C1(c2ncc(C(=O)NC34CC(c5nc(-c6ccc(Cl)cc6)cs5)(C3)C4)o2)CC1] | |
| | MS obsd. (ESI⁺) [(M + H)⁺]: 490.0. ¹H NMR (400 MHz, | |
| | DMSO-d₆) δ ppm: 9.05 (s, 1H), 8.67 (s, 1H), 8.11 (s, 1H), 7.97 | |
| | (d, J = 8.6 Hz, 2H), 7.50 (d, J = 8.6 Hz, 2H), 2.66-2.69 (m, 3H), | |
| | 2.27-2.37 (m, 6H), 1.75-1.79 (m, 2H), 1.69-1.73 (m, 2H). | |
| 7 | N-[3-[4-(4-chlorophenyl)thiazol-2-yl]-1- | Amine: Int-1 |
| | bicyclo[1.1.1]pentanyl]-5-(1-methylsulfonylcyclopropyl)-1,3,4- | Acid: Int-43 |
| | oxadiazole-2-carboxamide | |
| | 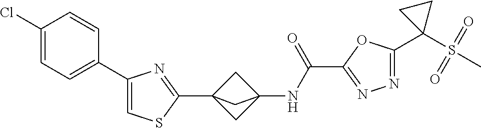 [SMILES: CS(=O)(=O)C1(c2nnc(C(=O)NC34CC(c5nc(-c6ccc(Cl)cc6)cs5)(C3)C4)o2)CC1] | |
| | MS obsd. (ESI⁺) [(M + H)⁺]: 491.0. ¹H NMR (400 MHz, CDCl₃) | |
| | δ ppm: 7.82 (d, J = 7.2 Hz, 2H), 7.54 (s, 1H), 7.37 (d, J = 7.2 | |
| | Hz, 2H), 3.29 (s, 3H), 2.68 (s, 6H), 2.04-2.08 (m, 2H), 1.78- | |
| | 1.82 (m, 2H). | |
| 8 | N-[3-[4-(3,4-dichlorophenyl)thiazol-2-yl]-1- | Amine: Int-4 |
| | bicyclo[1.1.1]pentanyl]-5-(1-methylsulfonylcyclopropyl)furan- | Acid: Int-37 |
| | 2-carboxamide | |
| | 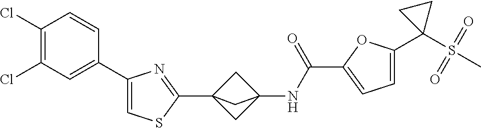 [SMILES: CS(=O)(=O)C1(c2ccc(C(=O)NC34CC(c5nc(-c6ccc(Cl)c(Cl)c6)cs5)(C3)C4)o2)CC1] | |
| | MS obsd. (ESI⁺) [(M + H)⁺]: 523.0. ¹H NMR (400 MHz, CDCl₃) | |
| | δ ppm: 8.00 (s, 1H), 7.70 (d, J = 8.4 Hz, 1H), 7.46 (d, J = 8.4 | |
| | Hz, 1H), 7.39 (s, 1H), 7.10 (d, J = 3.4 Hz, 1H), 6.82 (s, 1H), | |
| | 6.63 (d, J = 3.4 Hz, 1H), 2.91 (s, 3H), 2.66 (s, 6H), 1.86-1.90 | |
| | (m, 2H), 1.42-1.46 (m, 2H). | |
| 9 | N-[3-[4-(6-chloro-3-pyridyl)thiazol-2-yl]-1- | Amine: Int-5 |
| | bicyclo[1.1.1]pentanyl]-5-(1-methylsulfonylcyclopropyl)furan- | Acid: Int-37 |
| | 2-carboxamide | |
| | 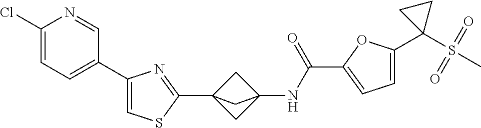 [SMILES: CS(=O)(=O)C1(c2ccc(C(=O)NC34CC(c5nc(-c6ccc(Cl)nc6)cs5)(C3)C4)o2)CC1] | |
| | MS obsd. (ESI⁺) [(M + H)⁺]: 490.1. ¹H NMR (400 MHz, CDCl₃) | |
| | δ ppm: 8.86 (d, J = 2.4 Hz, 1H), 8.18 (dd, J = 8.4, 2.4 Hz, 1H), | |
| | 7.48 (s, 1H), 7.38 (d, J = 8.4 Hz, 1H), 7.11 (d, J = 3.6 Hz, 1H), | |
| | 6.83 (s, 1H), 6.65 (d, J = 3.6 Hz, 1H), 2.93 (s, 3H), 2.67 (s, 6H), | |
| | 1.87-1.91 (m, 2H), 1.43-1.49 (m, 2H). | |
| 10 | N-[3-[4-(5-chloro-2-pyridyl)thiazol-2-yl]-1- | Amine: Int-6 |
| | bicyclo[1.1.1]pentanyl]-5-(1-methylsulfonylcyclopropyl)furan- | Acid: Int-37 |
| | 2-carboxamide | |
| | 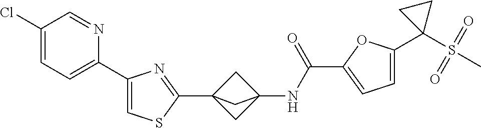 [SMILES: CS(=O)(=O)C1(c2ccc(C(=O)NC34CC(c5nc(-c6ccc(Cl)cn6)cs5)(C3)C4)o2)CC1] | |
| | MS obsd. (ESI⁺) [(M + H)⁺]: 490.1. ¹H NMR (400 MHz, CDCl₃) | |
| | δ ppm: 8.55 (d, J = 2.4 Hz, 1H), 8.10 (d, J = 8.4 Hz, 1H), 7.98 | |
| | (s, 1H), 7.73 (dd, J = 8.4, 2.4 Hz, 1H), 7.11 (d, J = 3.6 Hz, 1H), | |
| | 6.83 (s, 1H), 6.65 (d, J = 3.6 Hz, 1H), 2.93 (s, 3H), 2.67 (s, 6H), | |
| | 1.87-1.92 (m, 2H), 1.43-1.49 (m, 2H). | |
| 11 | 5-(1-methylsulfonylcyclopropyl)-N-[3-[4-(2-pyridyl)thiazol-2- | Amine: Int-7 |
| | yl]-1-bicyclo[1.1.1]pentanyl]furan-2-carboxamide | Acid: Int-37 |
| | 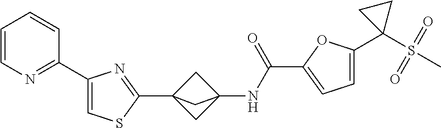 [SMILES: CS(=O)(=O)C1(c2ccc(C(=O)NC34CC(c5nc(-c6ccccn6)cs5)(C3)C4)o2)CC1] | |
| | MS obsd. (ESI⁺) [(M + H)⁺]: 456.0. ¹H NMR (400 MHz, | |
| | CD₃OD) δ ppm: 8.54-8.58 (m, 1H), 8.13 (d, J = 7.8 Hz, 1H), | |
| | 8.07 (s, 1H), 7.88-7.92 (m, 1H), 7.34-7.38 (m, 1H), 7.12 (d, J = | |
| | 3.6 Hz, 1H), 6.78 (d, J = 3.6 Hz, 1H), 3.03 (s, 3H), 2.64 (s, 6H), | |
| | 1.75-1.83 (m, 2H), 1.52-1.59 (m, 2H). | |
| 12 | 5-(1-methylsulfonylcyclopropyl)-N-[3-(4-pyrimidin-5- | Amine: Int-8 |
| | ylthiazol-2-yl)-1-bicyclo[1.1.1]pentanyl]furan-2-carboxamide | Acid: Int-37 |
| | 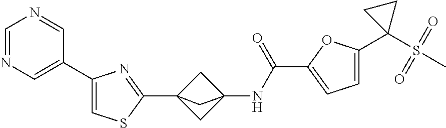 [SMILES: CS(=O)(=O)C1(c2ccc(C(=O)NC34CC(c5nc(-c6cncnc6)cs5)(C3)C4)o2)CC1] | |
| | MS obsd. (ESI⁺) [(M + H)⁺]: 457.0. ¹H NMR (400 MHz, | |
| | CD₃OD) δ ppm: 9.31 (s, 2H), 9.11 (s, 1H), 8.11 (s, 1H), 7.12 (d, | |
| | J = 3.6 Hz, 1H), 6.79 (d, J = 3.6 Hz, 1H), 3.02 (s, 3H), 2.64 (s, | |
| | 6H), 1.77-1.81 (m, 2H), 1.53-1.58 (m, 2H). | |
| 13 | 5-(1-methylsulfonylcyclopropyl)-N-[3-(4-phenylthiazol-2-yl)-1- | Amine: Int-9 |
| | bicyclo[1.1.1]pentanyl]furan-2-carboxamide | Acid: Int-37 |
| | 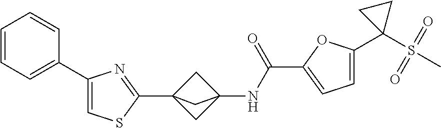 [SMILES: CS(=O)(=O)C1(c2ccc(C(=O)NC34CC(c5nc(-c6ccccc6)cs5)(C3)C4)o2)CC1] | |
| | MS obsd. (ESI⁺) [(M + H)⁺]: 455.3. ¹H NMR (400 MHz, | |
| | DMSO-d₆) δ ppm: 9.10 (s, 1H), 8.03 (s, 1H), 7.97-7.91 (m, | |
| | 2H), 7.47-7.41 (m, 2H), 7.37-7.32 (m, 1H), 7.12 (d, J = 3.2 Hz, | |
| | 1H), 6.79 (d, J = 3.2 Hz, 1H), 3.09 (s, 3H), 2.52 (s, 6H), 1.72- | |
| | 1.66 (m, 2H), 1.56-1.50 (m, 2H). | |
| 14 | N-[3-(4-cyclopropylthiazol-2-yl)-1-bicyclo[1.1.1]pentanyl]-5- | Amine: Int-10 |
| | (1-methylsulfonylcyclopropyl)furan-2-carboxamide | Acid: Int-37 |
| | 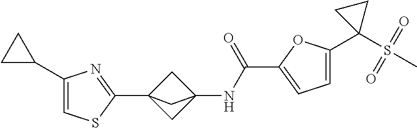 [SMILES: CS(=O)(=O)C1(c2ccc(C(=O)NC34CC(c5nc(C6CC6)cs5)(C3)C4)o2)CC1] | |
| | MS obsd. (ESI⁺) [(M + H)⁺]: 419.4. ¹H NMR (400 MHz, | |
| | DMSO-d₆) δ ppm: 9.05 (s, 1H), 7.14 (s, 1H), 7.10 (d, J = 3.2 | |
| | Hz, 1H), 6.78 (d, J = 3.2 Hz, 1H), 3.08 (s, 3H), 2.42 (s, 6H), | |
| | 2.08-2.00 (m, 1H), 1.71-1.65 (m, 2H), 1.55-1.50 (m, 2H), 0.90- | |
| | 0.83 (m, 2H), 0.78-0.72 (m, 2H). | |
| 15 | N-[3-(4-bromothiazol-2-yl)-1-bicyclo[1.1.1]pentanyl]-5-(1- | Amine: Int-11 |
| | methylsulfonylcyclopropyl)furan-2-carboxamide | Acid: Int-37 |
| | 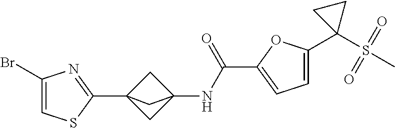 [SMILES: CS(=O)(=O)C1(c2ccc(C(=O)NC34CC(c5nc(Br)cs5)(C3)C4)o2)CC1] | |
| | MS obsd. (ESI⁺) [(M + H)⁺]: 458.9. ¹H NMR (400 MHz, | |
| | DMSO-d₆) δ ppm: 9.09 (s, 1H), 7.77 (s, 1H), 7.11 (d, J = 3.6 | |
| | Hz, 1H), 6.78 (d, J = 3.6 Hz, 1H), 3.08 (s, 3H), 2.47 (s, 6H), | |
| | 1.68-1.66 (m, 2H), 1.53-1.51 (m, 2H). | |
| 16 | 5-(1-methylsulfonylcyclopropyl)-N-[3-[4- | Amine: Int-12 |
| | (trifluoromethyl)thiazol-2-yl]-1-bicyclo[1.1.1]pentanyl]furan-2- | Acid: Int-37 |
| | carboxamide | |
| | 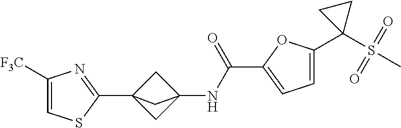 [SMILES: CS(=O)(=O)C1(c2ccc(C(=O)NC34CC(c5nc(C(F)(F)F)cs5)(C3)C4)o2)CC1] | |
| | MS obsd. (ESI⁺) [(M + H)⁺]: 447.3. ¹H NMR (400 MHz, | |
| | DMSO-d₆) δ ppm: 9.09 (s, 1H), 8.44 (s, 1H), 7.11 (d, J = 3.6 | |
| | Hz, 1H), 6.79 (d, J = 3.6 Hz, 1H), 3.08 (s, 3H), 2.49 (s, 6H), | |
| | 1.70-1.67 (m, 2H), 1.54-1.52 (m, 2H). | |
| 17 | 5-(1-methylsulfonylcyclopropyl)-N-[3-(3-phenyl-1,2,4- | Amine: Int-13 |
| | thiadiazol-5-yl)-1-bicyclo[1.1.1]pentanyl]furan-2-carboxamide | Acid: Int-37 |
| | 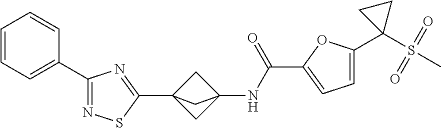 [SMILES: CS(=O)(=O)C1(c2ccc(C(=O)NC34CC(c5nc(-c6ccccc6)ns5)(C3)C4)o2)CC1] | |
| | MS obsd. (ESI⁺) [(M + H)⁺]: 456.1. ¹H NMR (400 MHz, | |
| | DMSO-d₆) δ ppm: 9.10 (s, 1H), 8.02 (d, J = 6.8 Hz, 2H), 7.54- | |
| | 7.67 (m, 3H), 7.13 (d, J = 3.6 Hz, 1H), 6.80 (d, J = 3.6 Hz, 1H), | |
| | 3.10 (s, 3H), 2.51 (s, 6H), 1.68-1.72 (m, 2H), 1.52-1.56 (m, | |
| | 2H). | |
| 18 | N-[3-[3-(4-chlorophenyl)-1,2,4-thiadiazol-5-yl]-1- | Amine: Int-14 |
| | bicyclo[1.1.1]pentanyl]-5-(1-methylsulfonylcyclopropyl)furan- | Acid: Int-37 |
| | 2-carboxamide | |
| | 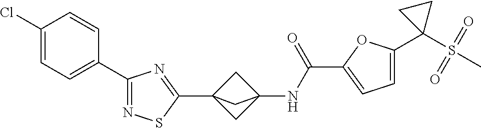 [SMILES: CS(=O)(=O)C1(c2ccc(C(=O)NC34CC(c5nc(-c6ccc(Cl)cc6)ns5)(C3)C4)o2)CC1] | |
| | MS obsd. (ESI⁺) [(M + H)⁺]: 490.0. ¹H NMR (400 MHz, | |
| | DMSO-d₆) δ ppm: 9.06 (s, 1 H), 8.02 (d, J = 8.6 Hz, 2 H), 7.63 | |
| | (d, J = 8.6 Hz, 2 H), 7.09 (d, J = 3.6 Hz, 1 H), 6.76 (d, J = 3.6 | |
| | Hz, 1 H), 3.07 (s, 3 H), 2.51 (s, 6 H), 1.64-1.68 (m, 2 H), 1.48- | |
| | 1.52 (m, 1 H). | |
| 19 | N-[3-[5-(4-chlorophenyl)thiazol-2-yl]-1- | Amine: Int-15 |
| | bicyclo[1.1.1]pentanyl]-5-(1-methylsulfonylcyclopropyl)furan- | Acid: Int-37 |
| | 2-carboxamide | |
| | 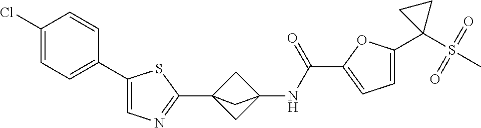 [SMILES: CS(=O)(=O)C1(c2ccc(C(=O)NC34CC(c5ncc(-c6ccc(Cl)cc6)s5)(C3)C4)o2)CC1] | |
| | MS obsd. (ESI⁺) [(M + H)⁺]: 489.0. ¹H NMR (400 MHz, | |
| | DMSO-d₆) δ ppm: 9.12 (s, 1H), 8.15 (s, 1H), 7.64-7.72 (m, | |
| | 2H), 7.46-7.55 (m, 2H), 7.13 (d, J = 3.6 Hz, 1H), 6.79 (d, J = | |
| | 3.6 Hz, 1H), 3.10 (s, 3H), 2.51 (s, 6H), 1.62-1.73 (m, 2H), 1.51- | |
| | 1.55 (m, 2H). | |
| 20 | N-[3-[5-(4-chlorophenyl)thiazol-2-yl]-1- | Amine: Int-15 |
| | bicyclo[1.1.1]pentanyl]-5-(1-methylsulfonylcyclopropyl)-1,3,4- | Acid: Int-43 |
| | oxadiazole-2-carboxamide | |
| | 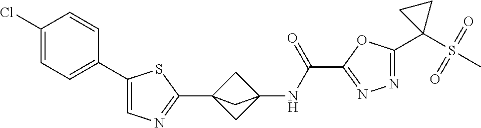 [SMILES: CS(=O)(=O)C1(c2nnc(C(=O)NC34CC(c5ncc(-c6ccc(Cl)cc6)s5)(C3)C4)o2)CC1] | |
| | MS obsd. (ESI⁺) [(M + H)⁺]: 491.3. ¹H NMR (400 MHz, CDCl₃) | |
| | δ ppm: 7.83 (s, 1H), 7.63 (s, 1H), 7.45 (d, J = 8.4 Hz, 2H), 7.36 | |
| | (d, J = 8.4 Hz, 2H), 3.29 (s, 3H), 2.67 (s, 6H), 2.04-2.08 (m, | |
| | 2H), 1.78-1.82 (m, 2H). | |
| 21 | N-[3-[5-(3,4-dichlorophenyl)thiazol-2-yl]-1- | Amine: Int-16 |
| | bicyclo[1.1.1]pentanyl]-5-(1-methylsulfonylcyclopropyl)furan- | Acid: Int-37 |
| | 2-carboxamide | |
| | 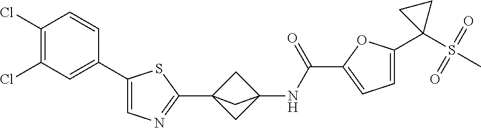 [SMILES: CS(=O)(=O)C1(c2ccc(C(=O)NC34CC(c5ncc(-c6ccc(Cl)c(Cl)c6)s5)(C3)C4)o2)CC1] | |
| | MS obsd. (ESI⁺) [(M + H)⁺]: 523.1. ¹H NMR (400 MHz, | |
| | DMSO-d₆) δ ppm: 9.11 (s, 1H), 8.26 (s, 1H), 7.99 (d, J = 2.0 | |
| | Hz, 1H), 7.69 (d, J = 8.4 Hz, 1H), 7.61 (dd, J = 8.4, 2.0 Hz, | |
| | 1H), 7.13 (d, J = 3.4 Hz, 1H), 6.80 (d, J = 3.4 Hz, 1H), 3.10 (s, | |
| | 3H), 2.51 (s, 6H), 1.62-1.81 (m, 2H), 1.52-1.56 (m, 2H). | |
| 22 | 5-(1-methylsulfonylcyclopropyl)-N-[3-(5-phenylthiazol-2-yl)-1- | Amine: Int-17 |
| | bicyclo[1.1.1]pentanyl]furan-2-carboxamide | Acid: Int-37 |
| | 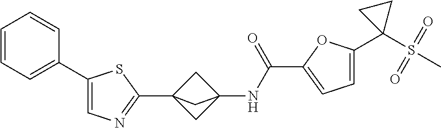 [SMILES: CS(=O)(=O)C1(c2ccc(C(=O)NC34CC(c5ncc(-c6ccccc6)s5)(C3)C4)o2)CC1] | |
| | MS obsd. (ESI⁺) [(M + H)⁺]: 455.2. ¹H NMR (400 MHz, | |
| | DMSO-d₆) δ ppm: 9.10 (s, 1H), 8.10 (s, 1H), 7.68-7.60 (m, | |
| | 2H), 7.47-7.40 (m, 2H), 7.38-7.33 (m, 1H), 7.12 (d, J = 3.2 Hz, | |
| | 1H), 6.79 (d, J = 3.2 Hz, 1H), 3.09 (s, 3H), 2.49 (S, 6H), 1.72- | |
| | 1.66 (m, 2H), 1.56-1.50 (m, 2H). | |
| 23 | N-[3-(5-cyclopropylthiazol-2-yl)-1-bicyclo[1.1.1]pentanyl]-5- | Amine: Int-18 |
| | (1-methylsulfonylcyclopropyl)furan-2-carboxamide | Acid: Int-37 |
| | 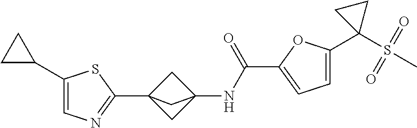 [SMILES: CS(=O)(=O)C1(c2ccc(C(=O)NC34CC(c5ncc(C6CC6)s5)(C3)C4)o2)CC1] | |
| | MS obsd. (ESI⁺) [(M + H)⁺]: 419.2. ¹H NMR (400 MHz, | |
| | DMSO-d₆) δ ppm: 9.04 (s, 1H), 7.42 (s, 1H), 7.10 (d, J = 3.2 | |
| | Hz, 1H), 6.78 (d, J = 3.2 Hz, 1H), 3.08 (s, 3H), 2.41 (s, 6H), | |
| | 2.14-2.08 (m, 1H), 1.70-1.66 (m, 2H), 1.54-1.49 (m, 2H), 1.03- | |
| | 0.98 (m, 2H), 0.68-0.63 (m, 2H). | |
| 24 | N-[3-(5-bromothiazol-2-yl)-1-bicyclo[1.1.1]pentanyl]-5-(1- | Amine: Int-19 |
| | methylsulfonylcyclopropyl)furan-2-carboxamide | Acid: Int-37 |
| | 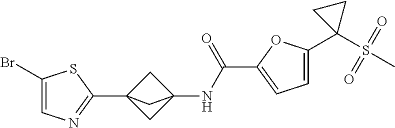 [SMILES: CS(=O)(=O)C1(c2ccc(C(=O)NC34CC(c5ncc(Br)s5)(C3)C4)o2)CC1] | |
| | MS obsd. (ESI⁺) [(M + H)⁺]: 458.9. ¹H NMR (400 MHz, | |
| | DMSO-d₆) δ ppm: 9.08 (s, 1H), 7.79 (s, 1H), 7.11 (d, J = 3.6 | |
| | Hz, 1H), 6.78 (d, J = 3.6 Hz, 1H), 3.08 (s, 3H), 2.46 (s, 6H), | |
| | 1.69-1.66 (m, 2H), 1.53-1.50 (m, 2H). | |
| 25 | 5-(1-methylsulfonylcyclopropyl)-N-[3-(5-phenyl-1,2,4- | Amine: Int-20 |
| | thiadiazol-3-yl)-1-bicyclo[1.1.1]pentanyl]furan-2-carboxamide | Acid: Int-37 |
| | 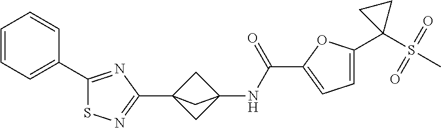 [SMILES: CS(=O)(=O)C1(c2ccc(C(=O)NC34CC(c5nsc(-c6ccccc6)n5)(C3)C4)o2)CC1] | |
| | MS obsd. (ESI⁺) [(M + H)⁺]: 456.0. ¹H NMR (400 MHz, | |
| | DMSO-d₆) δ ppm: 9.11 (s, 1H), 8.02 (d, J = 6.8 Hz, 2H), 7.52- | |
| | 7.68 (m, 3H), 7.13 (d, J = 3.6 Hz, 1H), 6.80 (d, J = 3.6 Hz, 1H), | |
| | 3.07 (s, 3H), 2.54 (s, 6H), 1.65-1.72 (m, 2H), 1.51-1.57 (m, | |
| | 2H). | |
| 26 | 5-(1-methylsulfonylcyclopropyl)-N-[3-(2-phenylthiazol-4-yl)-1- | Amine: Int-21 |
| | bicyclo[1.1.1]pentanyl]furan-2-carboxamide | Acid: Int-37 |
| | 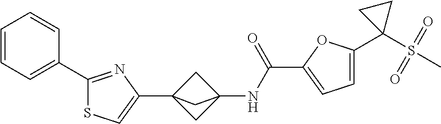 [SMILES: CS(=O)(=O)C1(c2ccc(C(=O)NC34CC(c5csc(-c6ccccc6)n5)(C3)C4)o2)CC1] | |
| | MS obsd. (ESI⁺) [(M + H)⁺]: 455.1. ¹H NMR (400 MHz, CDCl₃) | |
| | δ ppm: 7.96 (d, J = 7.6 Hz, 2H), 7.43-7.48 (m, 3H), 7.12 (d, J = | |
| | 3.6 Hz, 1H), 7.03 (s, 1H), 6.67 (d, J = 3.6 Hz, 1H), 3.01 (s, 3H), | |
| | 2.60 (s, 6H), 1.89-1.93 (d, J = 2.4 Hz, 2H), 1.47-1.51 (dd, J = | |
| | 7.6, 5.2 Hz, 2H). | |
| 27 | N-[3-[2-(4-chlorophenyl)thiazol-4-yl]-1- | Amine: Int-22 |
| | bicyclo[1.1.1]pentanyl]-5-(1-methylsulfonylcyclopropyl)furan- | Acid: Int-37 |
| | 2-carboxamide | |
| | 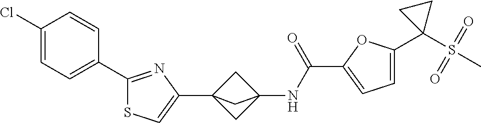 [SMILES: CS(=O)(=O)C1(c2ccc(C(=O)NC34CC(c5csc(-c6ccc(Cl)cc6)n5)(C3)C4)o2)CC1] | |
| | MS obsd. (ESI⁺) [(M + H)⁺]: 489.1. ¹H NMR (400 MHz, | |
| | DMSO-d₆) δ ppm: 9.04 (s, 1H), 7.91-7.99 (m, 2H), 7.54-7.61 | |
| | (m, 2H), 7.52 (s, 1H), 7.13 (d, J = 3.4 Hz, 1H), 6.79 (d, J = 3.4 | |
| | Hz, 1H), 3.11 (s, 3H), 2.43 (s, 6H), 1.68-1.72 (m, 2H), 1.52- | |
| | 1.57 (m, 2H). | |
| 28 | N-[3-[5-(4-chlorophenyl)-1,3,4-oxadiazol-2-yl]-1- | Amine: Int-23 |
| | bicyclo[1.1.1]pentanyl]-5-(1-methylsulfonylcyclopropyl)furan- | Acid: Int-37 |
| | 2-carboxamide | |
| | 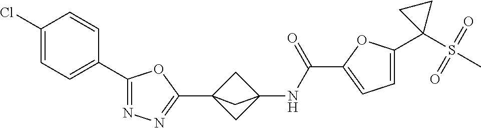 [SMILES: CS(=O)(=O)C1(c2ccc(C(=O)NC34CC(c5nnc(-c6ccc(Cl)cc6)o5)(C3)C4)o2)CC1] | |
| | MS obsd. (ESI⁺) [(M + H)⁺]: 474.0. ¹H NMR (400 MHz, | |
| | DMSO-d₆) δ ppm: 9.18 (s, 1H), 8.03 (d, J = 7.6 Hz, 2H), 7.67 | |
| | (d, J = 7.6 Hz, 2H), 7.13 (d, J = 3.6 Hz, 1H), 6.80 (d, J = 3.6 | |
| | Hz, 1H), 3.10 (s, 3H), 2.60 (s, 6H), 1.66-1.73 (m, 2H), 1.52- | |
| | 1.56 (m, 2H). | |
| 29 | N-[3-[1-(4-chlorophenyl)triazol-4-yl]-1- | Amine: Int-24 |
| | bicyclo[1.1.1]pentanyl]-5-(1-methylsulfonylcyclopropyl)furan- | Acid: Int-37 |
| | 2-carboxamide | |
| | 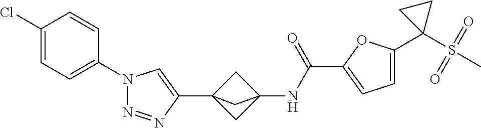 [SMILES: CS(=O)(=O)C1(c2ccc(C(=O)NC34CC(c5cn(-c6ccc(Cl)cc6)nn5)(C3)C4)o2)CC1] | |
| | MS obsd. (ESI⁺) [(M + H)⁺]: 473.1. ¹H NMR (400 MHz, CDCl₃) | |
| | δ ppm: 7.77 (s, 1H), 7.67 (d, J = 8.8 Hz, 2H), 7.50 (d, J = 8.8 | |
| | Hz, 2H), 7.10 (d, J = 3.6 Hz, 1H), 6.83 (s, 1H), 6.65 (d, J = 3.6 | |
| | Hz, 1H), 2.93 (s, 3H), 2.61 (s, 6H), 1.87-1.92 (m, 2H), 1.43- | |
| | 1.49 (m, 2H). | |
| 30 | N-[3-[2-(4-chlorophenyl)triazol-4-yl]-1- | Amine: Int-25 |
| | bicyclo[1.1.1]pentanyl]-5-(1-methylsulfonylcyclopropyl)furan- | Acid: Int-37 |
| | 2-carboxamide | |
| | 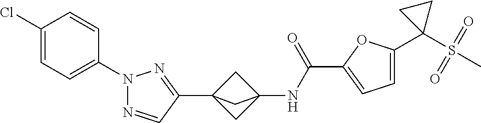 [SMILES: CS(=O)(=O)C1(c2ccc(C(=O)NC34CC(c5cnn(-c6ccc(Cl)cc6)n5)(C3)C4)o2)CC1] | |
| | MS obsd. (ESI⁺) [(M + H)⁺]: 473.1. ¹H NMR (400 MHz, CDCl₃) | |
| | δ ppm: 7.99 (d, J = 8.8 Hz, 2H), 7.62 (s, 1H), 7.43 (d, J = 8.8 | |
| | Hz, 2H), 7.10 (d, J = 3.6 Hz, 1H), 6.82 (s, 1H), 6.65 (d, J = 3.6 | |
| | Hz, 1H), 2.93 (s, 3H), 2.59 (s, 6H), 1.87-1.91 (m, 2H), 1.43- | |
| | 1.49 (m, 2H). | |
| 31 | N-[3-[2-(4-chlorophenyl)triazol-4-yl]-1- | Amine: Int-25 |
| | bicyclo[1.1.1]pentanyl]-1,1-dioxo-thiolane-3-carboxamide | Acid: 1,1- |
| | | dioxothiolane-3- |
| | | carboxylic acid |
| | 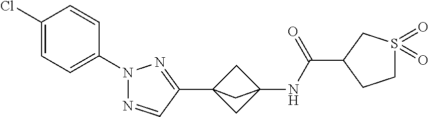 [SMILES: O=C(NC12CC(c3cnn(-c4ccc(Cl)cc4)n3)(C1)C2)C1CCS(=O)(=O)C1] | |
| | MS obsd. (ESI⁺) [(M + H)⁺]: 407.09. ¹H NMR (400 MHz, | |
| | CDCl₃) δ ppm: 7.95-8.00 (m, 2 H), 7.60 (s, 1 H), 7.40-7.45 (m, | |
| | 2 H), 6.08 (s, 1 H), 3.27-3.38 (m, 2 H), 3.21-3.25 (m, 1 H), | |
| | 3.01-3.13 (m, 2 H), 2.50 (s, 6 H), 2.40-2.44 (m, 2 H). | |
| 32 | N-[3-[3-(4-chlorophenyl)-1,2,4-oxadiazol-5-yl]-1- | Amine: Int-26 |
| | bicyclo[1.1.1]pentanyl]-5-(1-methylsulfonylcyclopropyl)furan- | Acid: Int-37 |
| | 2-carboxamide | |
| | 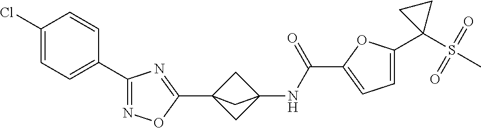 [SMILES: CS(=O)(=O)C1(c2ccc(C(=O)NC34CC(c5nc(-c6ccc(Cl)cc6)no5)(C3)C4)o2)CC1] | |
| | MS obsd. (ESI⁺) [(M + Na)⁺]: 496.0. ¹H NMR (400 MHz, | |
| | CDCl₃) δ ppm: 8.02 (d, J = 8.4 Hz, 2 H), 7.46 (d, J = 8.4 Hz, 2 | |
| | H), 7.14 (d, J = 3.2 Hz, 1 H), 6.96 (s,1 H), 6.65(d, J = 3.2 Hz, | |
| | 1 H), 2.93 (s, 3 H), 2.76 (s, 6 H), 1.88-1.92 (m, 2 H), 1.43-1.49 | |
| | (m, 2 H). | |
| 33 | N-[3-[3-(4-chlorophenyl)-1,2,4-oxadiazol-5-yl]-1- | Amine: Int-26 |
| | bicyclo[1.1.1]pentanyl]-3-(methylsulfonylmethyl)-1,2,4- | Acid: Int-38 |
| | thiadiazole-5-carboxamide | |
| | 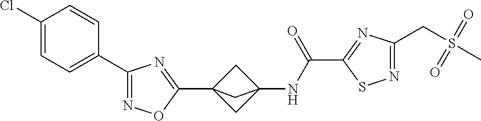 [SMILES: CS(=O)(=O)Cc1nsc(C(=O)NC23CC(c4nc(-c5ccc(Cl)cc5)no4)(C2)C3)n1] | |
| | MS obsd. (ESI⁺) [(M + H)⁺]: 466.2. ¹H NMR (400 MHz, CDCl₃) | |
| | δ ppm: 8.03 (d, J = 8.4 Hz, 2H), 7.72 (s, 1H), 7.47 (d, J = 8.4 | |
| | Hz, 2H), 4.72 (s, 2H), 3.10 (s, 3H), 2.80 (s, 6H). | |
| 34 | N-[3-[3-(4-chlorophenyl)-1,2,4-oxadiazol-5-yl]-1- | Amine: Int-26 |
| | bicyclo[1.1.1]pentanyl]-3-(1-methylsulfonylcyclopropyl)-1,2,4- | Acid: Int-39 |
| | thiadiazole-5-carboxamide | |
| | 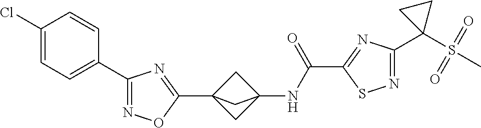 [SMILES: CS(=O)(=O)C1(c2nsc(C(=O)NC34CC(c5nc(-c6ccc(Cl)cc6)no5)(C3)C4)n2)CC1] | |
| | MS obsd. (ESI⁺) [(M + H)⁺]: 492.2. ¹H NMR (400 MHz, CDCl₃) | |
| | δ ppm: 8.02 (d, J = 8.6 Hz, 2H), 7.61 (s, 1H), 7.47 (d, J = 8.6 | |
| | Hz, 2H), 3.29 (s, 3H), 2.80 (s, 6H), 2.02-2.07 (m, 2H), 1.76- | |
| | 1.81 (m, 2H). | |
| 35 | N-[3-[3-(4-fluorophenyl)-1,2,4-oxadiazol-5-yl]-1- | Amine: Int-32 |
| | bicyclo[1.1.1]pentanyl]-3-(1-methylsulfonylcyclopropyl)-1,2,4- | Acid: Int-39 |
| | thiadiazole-5-carboxamide | |
| | 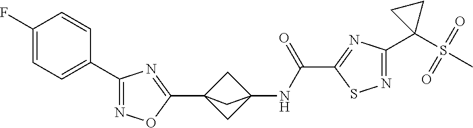 [SMILES: CS(=O)(=O)C1(c2nsc(C(=O)NC34CC(c5nc(-c6ccc(F)cc6)no5)(C3)C4)n2)CC1] | |
| | MS obsd. (ESI⁺) [(M + H)⁺]: 476.1. ¹H NMR (400 MHz, CDCl₃) | |
| | δ ppm: 8.08 (dd, J = 8.8, 5.4 Hz, 2H), 7.59 (s, 1H), 7.17 (t, J = | |
| | 8.8 Hz, 2H), 3.29 (s, 3H), 2.80 (s, 6H), 2.02-2.06 (m, 2H), 1.76- | |
| | 1.80 (m, 2H). | |
| 36 | N4-[3-[3-(4-chlorophenyl)-1,2,4-oxadiazol-5-yl]-1- | Amine: Int-26 |
| | bicyclo[1.1.1]pentanyl]pyridine-2,4-dicarboxamide | Acid: 2- |
| | | carbamoylpyridine- |
| | | 4-carboxylic acid |
| | 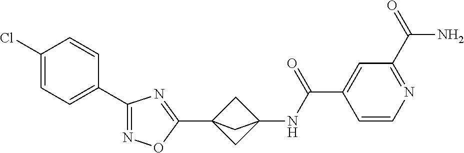 [SMILES: NC(=O)c1cc(C(=O)NC23CC(c4nc(-c5ccc(Cl)cc5)no4)(C2)C3)ccn1] | |
| | MS obsd. (ESI⁺) [(M + H)⁺]: 410.09. ¹H NMR (400 MHz, | |
| | CDCl₃) δ ppm: 8.77 (d, J = 5.0 Hz, 1 H), 8.46 (s, 1H), 8.00-7.92 | |
| | (m, 4 H), 7.46 (d, J = 8.4 Hz, 2 H), 7.13 (s, 1 H), 5.77 (s, 1 H), | |
| | 2.79 (s, 6 H). | |
| 37 | N-[3-[3-(4-chlorophenyl)-1,2,4-oxadiazol-5-yl]-1- | Amine: Int-26 |
| | bicyclo[1.1.1]pentanyl]-3-(1-methyl-2-methylsulfonyl- | Acid: Int-44 |
| | ethyl)benzamide | |
| | 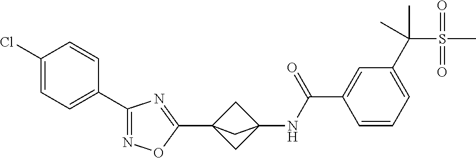 [SMILES: CC(C)(c1cccc(C(=O)NC23CC(c4nc(-c5ccc(Cl)cc5)no4)(C2)C3)c1)S(C)(=O)=O] | |
| | MS obsd. (ESI⁺) [(M + H)⁺]: 486.2. ¹H NMR (400 MHz, CDCl₃) | |
| | δ ppm: 7.99-8.08 (m, 3H), 7.84 (d, J = 8.0 Hz, 1H), 7.76 (d, J = | |
| | 8.0 Hz, 1H), 7.43-7.53 (m, 3H), 6.72 (s, 1H), 2.78 (s, 6H), 2.57 | |
| | (s, 3H), 1.90 (s, 6H). | |
| 38 | N-[3-[3-(4-chlorophenyl)-1,2,4-oxadiazol-5-yl]-1- | Amine: Int-26 |
| | bicyclo[1.1.1]pentanyl]-6-(1-methyl-1-methylsulfonyl- | Acid: Int-45 |
| | propyl)pyridine-2-carboxamide | |
| | 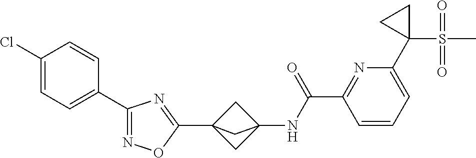 [SMILES: CS(=O)(=O)C1(c2cccc(C(=O)NC34CC(c5nc(-c6ccc(Cl)cc6)no5)(C3)C4)n2)CC1] | |
| | MS obsd. (ESI⁺) [(M + H)⁺]: 476.1. ¹H NMR (400 MHz, CDCl₃) | |
| | δ ppm: 8.13-8.33 (m, 2H), 8.05-7.88 (m, 4H), 7.43-7.47 (m, | |
| | 2H), 2.89 (s, 3H), 2.79 (s, 6H), 1.92-1.96 (m, 2H), 1.49-1.53 | |
| | (m, 2H). | |
| 39 | N-[3-[3-(4-chlorophenyl)-1,2,4-oxadiazol-5-yl]-1- | Amine: Int-26 |
| | bicyclo[1.1.1]pentanyl]-2-(1-methyl-1-methylsulfonyl- | Acid: Int-46 |
| | propyl)pyridine-4-carboxamide | |
| | 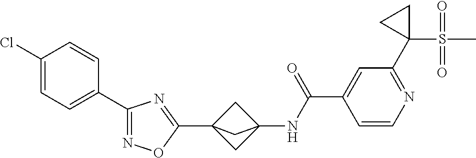 [SMILES: CS(=O)(=O)C1(c2cc(C(=O)NC34CC(c5nc(-c6ccc(Cl)cc6)no5)(C3)C4)ccn2)CC1] | |
| | MS obsd. (ESI⁺) [(M + H)⁺]: 485.10, ¹H NMR (400 MHz, | |
| | CDCl₃) δ ppm: 8.79 (s, 1 H), 8.21 (s, 1 H), 8.13 (s, 1 H), 8.02 | |
| | (d, J = 8.6 Hz, 2 H), 7.71 (s, 1 H), 7.46 (d, J = 8.6 Hz, 2 H), | |
| | 3.05 (s, 3 H), 2.81 (s, 6 H), 2.04 (s, 2 H), 1.66 (s, 2 H). | |
| 40 | N-[3-[5-(4-chlorophenyl)-1,2,4-oxadiazol-3-yl]-1- | Amine: Int-27 |
| | bicyclo[1.1.1]pentanyl]-5-(1-methylsulfonylcyclopropyl)furan- | Acid: Int-37 |
| | 2-carboxamide | |
| | 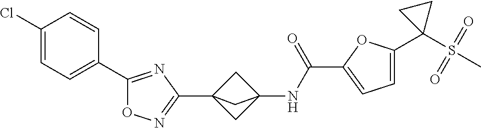 [SMILES: CS(=O)(=O)C1(c2ccc(C(=O)NC34CC(c5noc(-c6ccc(Cl)cc6)n5)(C3)C4)o2)CC1] | |
| | MS obsd. (ESI⁺) [(M + H)⁺]: 474.1. ¹H NMR (400 MHz, CDCl₃) | |
| | δ ppm: 8.08 (d, J = 8.6 Hz, 2H), 7.51 (d, J = 8.6 Hz, 2H), 7.11 | |
| | (d, J = 3.6 Hz, 1H), 6.97 (s, 1H), 6.64 (d, J = 3.6 Hz, 1H), 2.93 | |
| | (s, 3H), 2.68 (s, 6H), 1.87-1.91 (m, 2H), 1.43-1.49 (m, 2H). | |
| 41 | N-[3-[5-(4-chlorophenyl)-1,2,4-oxadiazol-3-yl]-1- | Amine: Int-27 |
| | bicyclo[1.1.1]pentanyl]-3-(1-methylsulfonylcyclopropyl)-1,2,4- | Acid: Int-39 |
| | thiadiazole-5-carboxamide | |
| | 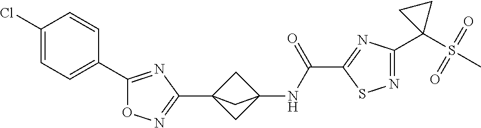 [SMILES: CS(=O)(=O)C1(c2nsc(C(=O)NC34CC(c5noc(-c6ccc(Cl)cc6)n5)(C3)C4)n2)CC1] | |
| | MS obsd. (ESI⁺) [(M + H)⁺]: 492.1. ¹H NMR (400 MHz, | |
| | DMSO-d₆) δ ppm: 9.96 (br, s 1H), 8.11 (d, J = 8.8 Hz, 2H), | |
| | 7.70 (d, J = 8.8 Hz, 2H), 3.44 (s, 3H), 2.58 (s, 6H), 1.86-1.83 | |
| | (m, 2H), 1.78-1.75 (m, 2H).. | |
| 42 | N-[3-[1-(4-chlorophenyl)pyrazol-3-yl]-1- | Amine: Int-28 |
| | bicyclo[1.1.1]pentanyl]-5-(1-methylsulfonylcyclopropyl)furan- | Acid: Int-37 |
| | 2-carboxamide | |
| | 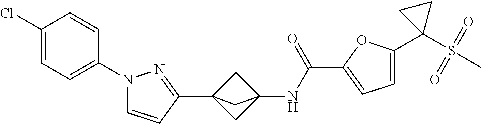 [SMILES: CS(=O)(=O)C1(c2ccc(C(=O)NC34CC(c5ccn(-c6ccc(Cl)cc6)n5)(C3)C4)o2)CC1] | |
| | MS obsd. (ESI⁺) [(M + H)⁺]: 472.1. ¹H NMR (400 MHz, CDCl₃) | |
| | δ ppm: 7.79 (d, J = 2.4 Hz, 1H), 7.57-7.63 (m, 2H), 7.36-7.42 | |
| | (m, 2H), 7.10 (d, J = 3.6 Hz, 1H), 6.64 (d, J = 3.6 Hz, 1H), 6.33 | |
| | (d, J = 2.4 Hz, 1H), 2.93 (s, 3H), 2.55 (s, 6H), 1.85-1.91 (m, | |
| | 2H), 1.43-1.49 (m, 2H). | |
| 43 | N-[3-[1-(4-chlorophenyl)pyrazol-3-yl]-1- | Amine: Int-28 |
| | bicyclo[1.1.1]pentanyl]-3-(1-methylsulfonylcyclopropyl)-1,2,4- | Acid: Int-39 |
| | thiadiazole-5-carboxamide | |
| | 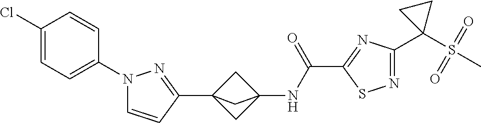 [SMILES: CS(=O)(=O)C1(c2nsc(C(=O)NC34CC(c5ccn(-c6ccc(Cl)cc6)n5)(C3)C4)n2)CC1] | |
| | MS obsd. (ESI⁺) [(M + H)⁺]: 490.0. ¹H NMR (400 MHz, | |
| | DMSO-d₆) δ ppm: 9.84 (s, 1H), 8.44 (d, J = 2.4 Hz, 1H), 7.84 | |
| | (d, J = 8.8 Hz, 2H), 7.55(d, J = 8.8 Hz, 2H), 6.48 (d, J = 2.0 | |
| | Hz, 1H), 3.44 (s, 3H), 2.45 (s, 6H), 1.83-1.77 (m, 4H). | |
| 44 | N-[3-[2-(4-chlorophenyl)tetrazol-5-yl]-1- | Amine: Int-29 |
| | bicyclo[1.1.1]pentanyl]-5-(1-methylsulfonylcyclopropyl)furan- | Acid: Int-37 |
| | 2-carboxamide | |
| | 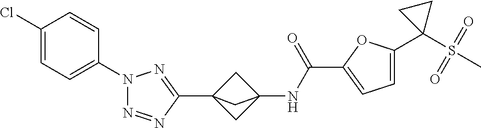 [SMILES: CS(=O)(=O)C1(c2ccc(C(=O)NC34CC(c5nnn(-c6ccc(Cl)cc6)n5)(C3)C4)o2)CC1] | |
| | MS obsd. (ESI⁺) [(M + H)⁺]: 474.1. ¹H NMR (400 MHz, CDCl₃) | |
| | δ ppm: 8.07 (d, J = 8.8 Hz, 2H), 7.53 (d, J = 8.8 Hz, 2H), 7.11 | |
| | (d, J = 3.6 Hz, 1H), 6.88 (s, 1H), 6.65 (d, J = 3.6 Hz, 1H), 2.93 | |
| | (s, 3H), 2.73 (s, 6H), 1.87-1.91 (m, 2H), 1.43-1.49 (m, 2H). | |
| 45 | N-[3-[4-(4-chlorophenyl)pyrimidin-2-yl]-1- | Amine: Int-30 |
| | bicyclo[1.1.1]pentanyl]-5-(1-methylsulfonylcyclopropyl)furan- | Acid: Int-37 |
| | 2-carboxamide | |
| | 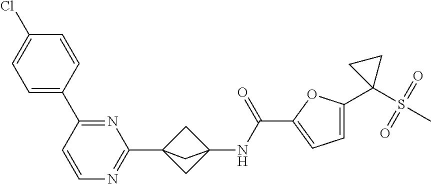 [SMILES: CS(=O)(=O)C1(c2ccc(C(=O)NC34CC(c5nccc(-c6ccc(Cl)cc6)n5)(C3)C4)o2)CC1] | |
| | MS obsd. (ESI⁺) [(M + H)⁺]: 484.3. ¹H NMR (400 MHz, CDCl₃) | |
| | δ ppm: 8.76 (d, J = 5.2 Hz, 1H), 8.11 (d, J = 8.2 Hz, 2H), 7.63 | |
| | (d, J = 5.2 Hz, 1H), 7.51 (d, J = 8.2 Hz, 2H), 7.12 (d, J = 3.2 | |
| | Hz, 1H), 6.84 (s, 1H), 6.66 (d, J = 3.2 Hz, 1H), 2.94 (s, 3H), | |
| | 2.74 (s, 6H), 1.87-1.91 (m, 2H), 1.46-1.50 (m, 2H). | |
| 46 | N-[3-[4-(4-chlorophenyl)pyrimidin-2-yl]-1- | Amine: Int-30 |
| | bicyclo[1.1.1]pentanyl]-3-(1-methylsulfonylcyclopropyl)-1,2,4- | Acid: Int-39 |
| | thiadiazole-5-carboxamide | |
| | 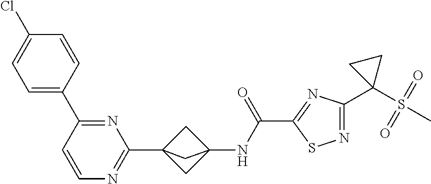 [SMILES: CS(=O)(=O)C1(c2nsc(C(=O)NC34CC(c5nccc(-c6ccc(Cl)cc6)n5)(C3)C4)n2)CC1] | |
| | MS obsd. (ESI⁺) [(M + H)⁺]: 502.1. ¹H NMR (400 MHz, CDCl₃) | |
| | δ ppm: 8.76 (s, 1H), 8.09 (d, J = 8.1 Hz, 2H), 7.58 (s, 1H), 7.50 | |
| | (d, J = 7.9 Hz, 2H), 3.31 (s, 3H), 2.74 (s, 6H), 2.02 (d, J = 12.0 | |
| | Hz, 2H), 1.80 (s, 2H). | |
| 47 | N-[3-[2-(4-chlorophenyl)pyrimidin-4-yl]-1- | Amine: Int-31 |
| | bicyclo[1.1.1]pentanyl]-5-(1-methylsulfonylcyclopropyl)furan- | Acid: Int-37 |
| | 2-carboxamide | |
| | 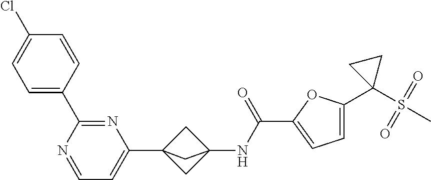 [SMILES: CS(=O)(=O)C1(c2ccc(C(=O)NC34CC(c5ccnc(-c6ccc(Cl)cc6)n5)(C3)C4)o2)CC1] | |
| | MS obsd. (ESI⁺) [(M + H)⁺]: 484.2. ¹H NMR (400 MHz, | |
| | DMSO-d₆) δ ppm: 9.08 (s, 1H), 8.84 (d, J = 5.2 Hz, 1H), 8.40- | |
| | 8.42 (m, 2H), 7.59-7.62 (m, 2H), 7.43 (d, J = 5.2 Hz, 1H), 7.13 | |
| | (d, J = 3.6 Hz, 1H), 6.80 (d, J = 3.6 Hz, 1H), 3.21-2.85 (m, 3H), | |
| | 2.49 (s, 6H), 1.67-1.71 (m, 2H), 1.52-1.56 (m, 2H). | |
| 48 | N-[3-[2-(4-chlorophenyl)pyrimidin-4-yl]-1- | Amine: Int-31 |
| | bicyclo[1.1.1]pentanyl]-3-(1-methylsulfonylcyclopropyl)-1,2,4- | Acid: Int-39 |
| | thiadiazole-5-carboxamide | |
| | 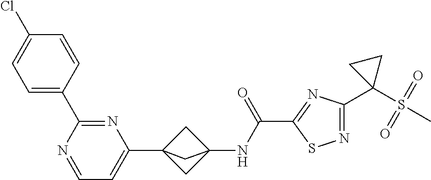 [SMILES: CS(=O)(=O)C1(c2nsc(C(=O)NC34CC(c5ccnc(-c6ccc(Cl)cc6)n5)(C3)C4)n2)CC1] | |
| | MS obsd. (ESI⁺) [(M + H)⁺]: 502.1. ¹H NMR (400 MHz, CDCl₃) | |
| | δ ppm: 8.73 (br, s, 1H), 8.42 (d, J = 7.8 Hz, 2H), 7.64 (br, s, | |
| | 1H), 7.46 (d, J = 7.8 Hz, 2H), 7.12 (d, J = 3.4 Hz, 1H), 3.30 (s, | |
| | 3H), 2.65 (s, 6H), 2.02-2.06 (m, 2H), 1.77-1.81 (m, 2H). | |
| 49 | N-[3-[5-bromo-2-(4-chlorophenyl)triazol-4-yl]-1- | Amine: Int-33 |
| | bicyclo[1.1.1]pentanyl]-5-(1-methylsulfonylcyclopropyl)furan- | Acid: Int-37 |
| | 2-carboxamide | |
| | 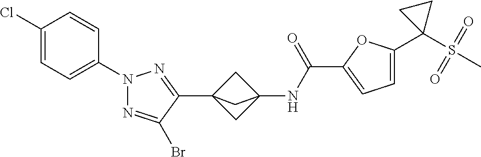 [SMILES: CS(=O)(=O)C1(c2ccc(C(=O)NC34CC(c5nn(-c6ccc(Cl)cc6)nc5Br)(C3)C4)o2)CC1] | |
| | MS obsd. (ESI⁺) [(M + H)⁺]: 551.1. ¹H NMR (400 MHz, CDCl₃) | |
| | δ ppm: 7.91-7.97 (m, 2H), 7.39-7.47 (m, 2H), 7.11 (d, J = 3.4 | |
| | Hz, 1H), 6.81 (s, 1H), 6.65 (d, J = 3.4 Hz, 1H), 2.93 (s, 3H), | |
| | 2.69 (s, 6H), 1.88-1.91 (m, 2H), 1.44-1.49 (m, 2H). | |
| 50 | N-[3-[2-(4-chlorophenyl)-5-methyl-triazol-4-yl]-1- | Amine: Int-34 |
| | bicyclo[1.1.1]pentanyl]-5-(1-methylsulfonylcyclopropyl)furan- | Acid: Int-37 |
| | 2-carboxamide | |
| | 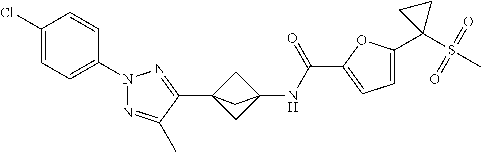 [SMILES: Cc1nn(-c2ccc(Cl)cc2)nc1C12CC(NC(=O)c3ccc(C4(S(C)(=O)=O)CC4)o3)(C1)C2] | |
| | MS obsd. (ESI⁺) [(M + H)⁺]: 487.1. ¹H NMR (400 MHz, CDCl₃) | |
| | δ ppm: 7.88-7.96 (m, 2H), 7.37-7.45 (m, 2H), 7.11 (d, J = 3.6 | |
| | Hz, 1H), 6.79 (s, 1H), 6.65 (d, J = 3.6 Hz, 1H), 2.93 (s, 3H), | |
| | 2.62 (s, 6H), 2.38 (s, 3H), 1.87-1.91 (m, 2H), 1.43-1.48 (m, | |
| | 2H). | |
| 51 | 3-(1-methylsulfonylcyclopropyl)-N-[3-[2-(p-tolyl)triazol-4-yl]- | Amine: Int-35 |
| | 1-bicyclo[1.1.1]pentanyl]-1,2,4-thiadiazole-5-carboxamide | Acid: Int-39 |
| | 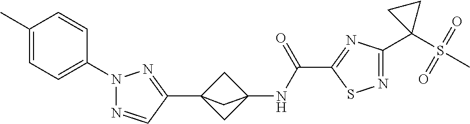 | |
| | MS obsd. (ESI⁺) [(M + H)⁺]: 471.2. ¹H NMR (400 MHz, CDCl₃) | |
| | δ ppm: 7.92 (d, J = 8.0 Hz, 2H), 7.61 (s, 1H), 7.53 (s, 1H), 7.28- | |
| | 7.18 (d, J = 8.4 Hz, 2H), 3.31 (s, 3H), 2.63 (s, 6H), 2.41 (s,3H), | |
| | 2.06-2.03 (m, 2H), 1.81-1.57 (m, 2H). | |
| 52 | N-[3-[2-(4-methoxyphenyl)triazol-4-yl]-1- | Amine: Int-36 |
| | bicyclo[1.1.1]pentanyl]-3-(1-methylsulfonylcyclopropyl)-1,2,4- | Acid: Int-37 |
| | thiadiazole-5-carboxamide | |
| | 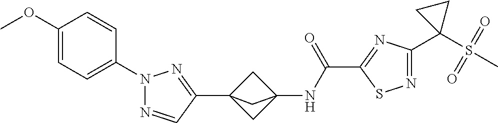 | |
| | MS obsd. (ESI⁺) [(M + H)⁺]: 487.1. ¹H NMR (400 MHz, CDCl₃) | |
| | δ ppm: 7.95-7.93 (m, 2H), 7.60 (s, 1H), 7.56 (s, 1H), 7.00-6.98 | |
| | (m, 2H), 3.87 (s, 3H), 3.31 (s, 3H), 2.63 (s, 6H), 2.06-2.02 (m, | |
| | 2H), 1.81-1.78 (m, 2H). | |

### Example 53

### N-[3-[4-(4-chlorophenyl)thiazol-2-yl]-1-bicyclo[1.1.1]pentanyl]-3-(1-methylsulfonylcyclopropyl)pyrazole-1-carboxamide

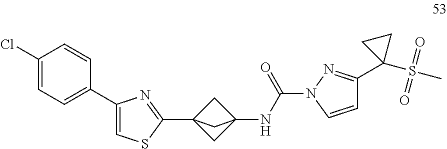 [SMILES: CS(=O)(=O)C1(c2ccn(C(=O)NC34CC(c5nc(-c6ccc(Cl)cc6)cs5)(C3)C4)n2)CC1]

The title compound was prepared according to the following scheme:

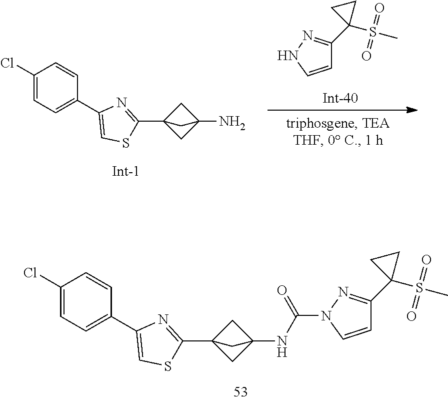 [SMILES: NC12CC(c3nc(-c4ccc(Cl)cc4)cs3)(C1)C2 ; CS(=O)(=O)C1(c2cc[nH]n2)CC1 ; CS(=O)(=O)C1(c2ccn(C(=O)NC34CC(c5nc(-c6ccc(Cl)cc6)cs5)(C3)C4)n2)CC1]

To a solution of triphosgene (139.38 mg, 0.47 mmol) in THF (10 mL) were added 3-[4-(4-chlorophenyl)thiazol-2-yl]bicyclo[1.1.1]pentan-1-amine (Int-1, 130.0 mg, 0.47 mmol) and TEA (284.82 mg, 2.82 mmol) at 0° C. under nitrogen atmosphere. After being stirred at 0° C. for 1 h, 3-(1-methylsulfonylcyclopropyl)-1H-pyrazole (Int-40, 87.47 mg, 0.47 mmol) was added followed by stirring at 0° C. for 1 h. The resulting solution was diluted with EtOAc (60 mL) and washed with brine (30 mL). The organic layer was dried over anhydrous sodium sulfate and concentrated under reduced pressure. The residue was purified by prep-HPLC (Kromasil-C18, 100×21.2 mm, 5 m; Mobile Phase: ACN—H₂O (0.1% FA), Gradient: 50-60) to give N-[3-[4-(4-chlorophenyl)thiazol-2-yl]-1-bicyclo[1.1.1]pentanyl]-3-(1-methylsulfonylcyclopropyl)pyrazole-1-carboxamide (Example 53, 105.7 mg, 46.02%) as a white solid. MS obsd. (ESI⁺) [(M+H)⁺]: 489.0. ¹H NMR (400 MHz, CDCl₃) δ ppm: 8.23 (d, J=2.8 Hz, 1H), 7.84 (d, J=8.4 Hz, 2H), 7.51 (s, 1H), 7.36-7.42 (m, 2H), 6.73 (d, J=2.8 Hz, 1H), 2.92 (s, 3H), 2.68 (s, 6H), 1.84-1.88 (m, 2H), 1.47-1.51 (m, 2H).

### Example 54

### N-[3-[5-(4-chlorophenyl)thiazol-2-yl]-1-bicyclo[1.1.1]pentanyl]-3-(1-methylsulfonylcyclopropyl)pyrazole-1-carboxamide

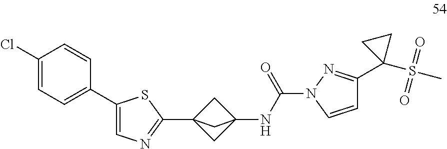 [SMILES: CS(=O)(=O)C1(c2ccn(C(=O)NC34CC(c5ncc(-c6ccc(Cl)cc6)s5)(C3)C4)n2)CC1]

The title compound was prepared in analogy to the procedure described for the preparation of Example 53, by using 3-[5-(4-chlorophenyl)thiazol-2-yl]bicyclo[1.1.1]pentan-1-amine (Int-15) instead of 3-[4-(4-chlorophenyl)thiazol-2-yl]bicyclo[1.1.1]pentan-1-amine (Int-1). The product was purified by preparative HPLC to afford Example 54 as a white solid. MS obsd. (ESI⁺) [(M+H)⁺]: 489.3. ¹H NMR (400 MHz, CDCl₃) δ ppm: 8.20 (d, J=2.8 Hz, 1H), 7.83 (s, 1H), 7.51 (s, 1H), 7.44 (d, J=8.8 Hz, 2H), 7.36 (d, J=8.8 Hz, 2H), 6.70 (d, J=2.8 Hz, 1H), 3.24 (s, 3H), 2.72 (s, 6H), 1.91-1.95 (m, 2H), 1.62-1.66 (m, 2H).

### Example 55

### N-[3-[5-(4-chlorophenyl)thiazol-2-yl]-1-bicyclo[1.1.1]pentanyl]-2-(1-methylsulfonylcyclopropyl)oxazole-5-carboxamide

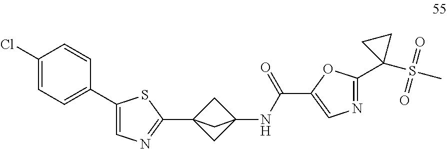 [SMILES: CS(=O)(=O)C1(c2ncc(C(=O)NC34CC(c5ncc(-c6ccc(Cl)cc6)s5)(C3)C4)o2)CC1]

The title compound was prepared according to the following scheme:

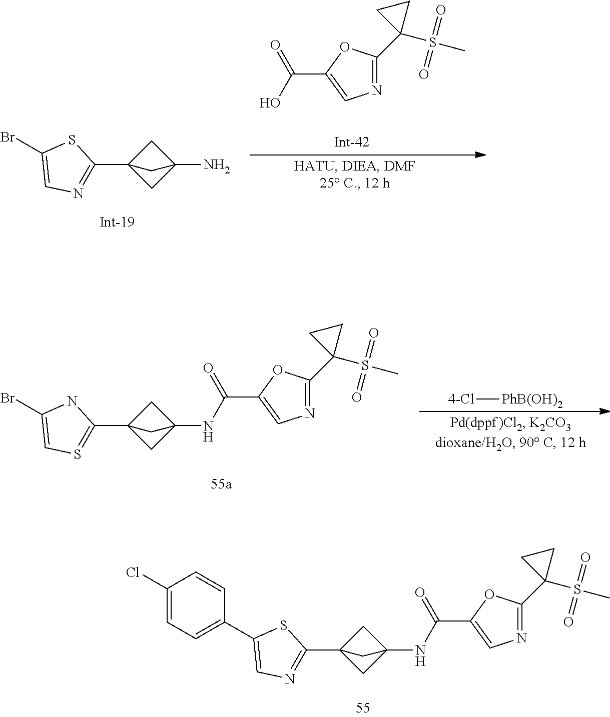

### Step 1: Preparation of N-[3-(5-bromothiazol-2-yl)-1-bicyclo[1.1.1]pentanyl]-2-(1-methylsulfonylcyclopropyl)oxazole-5-carboxamide (55a)

To a mixture of 3-(5-bromothiazol-2-yl)bicyclo[1.1.1]pentan-1-amine (Int-19, 200 mg, 0.80 mmol) and DIEA (0.2 mL, 4.9 mmol) in 10 mL of DMF was added HATU (744 mg, 2.0 mmol), followed by 2-(methylsulfonylmethyl)oxazole-5-carboxylic acid (Int-42, 184.4 mg, 0.80 mmol). After being stirred at 25° C. overnight, the resulting solution was diluted with ethyl acetate (20 mL) and washed with brine (10 mL). The organic layer was dried over anhydrous sodium sulfate and concentrated under reduced pressure. The residue was purified by flash column (eluting with EtOAc/PE=4/1) to give N-[3-(5-bromothiazol-2-yl)-1-bicyclo[1.1.1]pentanyl]-2-(1-methylsulfonylcyclopropyl)oxazole-5-carboxamide (55a, 169 mg, 46.2%) as a light yellow solid. MS obsd. (ESI⁺) [(M+H)⁺]: 459.0.

### Step 2: Preparation of N-[3-[5-(4-chlorophenyl)thiazol-2-yl]-1-bicyclo[1.1.1]pentanyl]-2-(1-methylsulfonylcyclopropyl)oxazole-5-carboxamide (Example 55)

The mixture of N-[3-(5-bromothiazol-2-yl)-1-bicyclo[1.1.1]pentanyl]-2-(1-methylsulfonylcyclopropyl)oxazole-5-carboxamide (55a, 133.0 mg, 0.29 mmol), 4-chlorophenylboronic acid (54.45 mg, 0.35 mmol), potassium carbonate (100.26 mg, 0.73 mmol) and Pd(dppf)Cl₂ (22.63 mg, 0.03 mmol) in 1,4-dioxane (5 mL) and water (1 mL) was stirred at 90° C. overnight under nitrogen atmosphere. After being cooled to room temperature, the resulting solution was diluted with ethyl acetate (30 mL) and filtered by Celite. The filtrate was washed with brine (3×20 mL), dried over anhydrous sodium sulfate and concentrated under reduced pressure. The residue was purified by prep-HPLC (Chromatographic columns: Kromasil-C18, 100×21.2 mm, 5 m; Mobile Phase: ACN—H₂O (0.1% FA), Gradient: 30-40) to give N-[3-[5-(4-chlorophenyl)thiazol-2-yl]-1-bicyclo[1.1.1]pentanyl]-2-(1-methylsulfonylcyclopropyl)oxazole-5-carboxamide (Example 55, 2.3 mg) as a white solid. MS obsd. (ESI⁺) [(M+H)⁺]: 490.0. ¹HNMR (MHz, CDCl₃) δ ppm: 8.20 (s, 1H), 7.83 (s, 1H), 7.46 (d, J=8.4 Hz, 2H), 7.37 (d, J=8.4 Hz, 2H), 3.21 (s, 3H), 2.66 (s, 6H), 1.94-1.99 (m, 2H), 1.67-1.71 (m, 2H).

### Example 56

### N-[3-[5-(6-chloro-3-pyridyl)thiazol-2-yl]-1-bicyclo[1.1.1]pentanyl]-5-(1-methylsulfonylcyclopropyl)furan-2-carboxamide

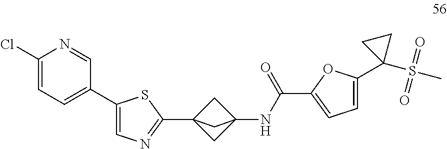 [SMILES: CS(=O)(=O)C1(c2ccc(C(=O)NC34CC(c5ncc(-c6ccc(Cl)nc6)s5)(C3)C4)o2)CC1]

The title compound was prepared in analogy to the procedure described for the preparation of Example 55, by using 5-(1-methylsulfonylcyclopropyl)furan-2-carboxylic acid (Int-37) instead of 2-(methylsulfonylmethyl)oxazole-5-carboxylic acid (Int-42) and 2-chloropyridine-5-boronic acid instead of 4-chlorophenylboronic acid. The product was purified by preparative HPLC to afford Example 56 as a white solid. MS obsd. (ESI⁺) [(M+H)⁺]: 490.0. ¹H NMR (400 MHz, DMSO-d₆) δ ppm: 9.10 (s, 1H), 8.73 (d, J=2.4 Hz, 1H), 8.28 (s, 1H), 8.15 (dd, J=8.4, 2.4 Hz, 1H), 7.61 (d, J=8.4 Hz, 1H), 7.12 (d, J=3.6 Hz, 1H), 6.79 (d, J=3.6 Hz, 1H), 3.09 (s, 3H), 2.51 (s, 6H), 1.67-1.71 (m, 2H), 1.51-1.55 (m, 2H).

### Example 57

### N-[3-[5-(5-chloro-2-pyridyl)thiazol-2-yl]-1-bicyclo[11.11]pentanyl]-5-(1-methylsulfonylcyclopropyl)furan-2-carboxamide

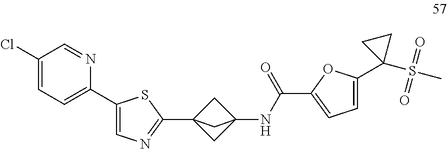 [SMILES: CS(=O)(=O)C1(c2ccc(C(=O)NC34CC(c5ncc(-c6ccc(Cl)cn6)s5)(C3)C4)o2)CC1]

The title compound was prepared in analogy to the procedure described for the preparation of Example 55, by using 5-(1-methylsulfonylcyclopropyl)furan-2-carboxylic acid (Int-37) instead of 2-(methylsulfonylmethyl)oxazole-5-carboxylic acid (Int-42) and tributyl-(5-chloro-2-pyridyl)stannane instead of 4-chlorophenylboronic acid. The product was purified by preparative HPLC to afford Example 57 as a white solid. MS obsd. (ESI⁺) [(M+H)⁺]: 490.0. ¹H NMR (400 MHz, DMSO-d₆) δ ppm: 9.10 (s, 1H), 8.61 (d, J=1.2 Hz, 1H), 8.43 (s, 1H), 7.98-8.10 (m, 2H), 7.12 (d, J=3.6 Hz, 1H), 6.79 (d, J=3.6 Hz, 1H), 3.09 (s, 3H), 2.56 (s, 6H), 1.67-1.71 (m, 2H), 1.51-1.55 (m, 2H).

### Example 58

### N-[3-[5-(5-chloropyrimidin-2-yl)thiazol-2-yl]-1-bicyclo[1.1.1]pentanyl]-5-(1-methylsulfonylcyclopropyl)furan-2-carboxamide

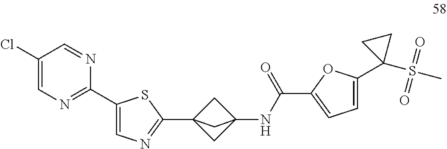 [SMILES: CS(=O)(=O)C1(c2ccc(C(=O)NC34CC(c5ncc(-c6ncc(Cl)cn6)s5)(C3)C4)o2)CC1]

The title compound was prepared according to the following scheme:

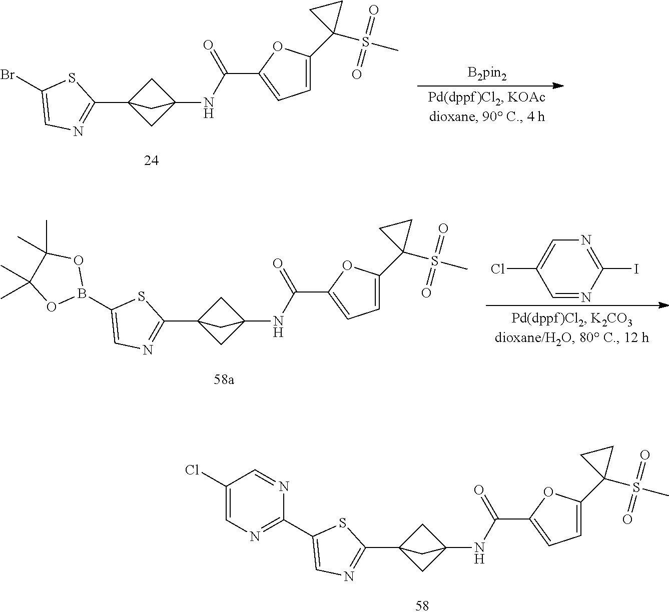

### Step 1: Preparation of 5-(1-methylsulfonylcyclopropyl)-N-[3-[5-(4,4,5,5-tetramethyl-1,3,2-dioxaborolan-2-yl)thiazol-2-yl]-1-bicyclo[1.1.1]pentanyl]furan-2-carboxamide (58a)

To a solution of mixture of N-[3-(5-bromothiazol-2-yl)-1-bicyclo[1.1.1]pentanyl]-5-(1-methylsulfonylcyclopropyl)furan-2-carboxamide (Example 24, 300 mg, 0.66 mmol) in 10 mL of dioxane were added bis(pinacolato)diboron (199.9 mg, 0.79 mmol), KOAc (193.12 mg, 1.97 mmol) and Pd(dppf)Cl₂ (48.0 mg, 0.07 mmol). After being heated with stirring at 90° C. for 4 h under N₂ atmosphere, the resulting mixture was used for the next step without purification. MS obsd. (ESI⁺) [(M+H)⁺]: 423.0.

### Step 2: Preparation of N-[3-[5-(5-chloropyrimidin-2-yl)thiazol-2-yl]-1-bicyclo[1.1.1]pentanyl]-5-(1-methylsulfonylcyclopropyl)furan-2-carboxamide (Example 58)

To a solution of 5-(1-methylsulfonylcyclopropyl)-N-[3-[5-(4,4,5,5-tetramethyl-1,3,2-dioxaborolan-2-yl)thiazol-2-yl]-1-bicyclo[1.1.1]pentanyl]furan-2-carboxamide (58a, prepared in Step 1) in dioxane (10 mL) and water (0.5 mL) were added 5-chloro-2-iodopyrimidine (53.62 mg, 0.22 mmol), K₂CO₃ (51.37 mg, 0.37 mmol) and Pd(dppf)Cl₂ (10.88 mg, 0.01 mmol). After being stirred at 80° C. for 12 h under N₂ atmosphere, the reaction mixture was extracted with water (2×20 mL) and EtOAc (2×20 mL). The combined organic layer was dried and concentrated in vacuo to give the crude product, which was further purified by prep-HPLC (Chromatographic columns: Kromasil-C18, 100×21.2 mm, 5 m; Mobile Phase: ACN—H₂O (0.1% TFA), Gradient: 35-45) to give N-[3-[5-(5-chloropyrimidin-2-yl)thiazol-2-yl]-1-bicyclo[1.1.1]pentanyl]-5-(1-methylsulfonylcyclopropyl)furan-2-carboxamide (Example 58, 12.4 mg) as a white solid. MS obsd. (ESI⁺) [(M+H)⁺]: 491.0. ¹H NMR (400 MHz, DMSO-d₆) δ ppm: 9.12 (s, 1H), 8.96 (s, 2H), 8.45 (s, 1H), 7.12 (d, J=3.4 Hz, 1H), 6.79 (d, J=3.4 Hz, 1H), 3.10 (s, 3H), 2.53 (s, 6H), 1.67-1.71 (m, 2H), 1.51-1.55 (m, 2H).

### Example 59

### N-[3-[5-(5-chloropyrazin-2-yl)thiazol-2-yl]-1-bicyclo[1.1.1]pentanyl]-5-(1-methylsulfonylcyclopropyl)furan-2-carboxamide

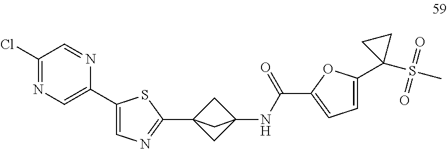 [SMILES: CS(=O)(=O)C1(c2ccc(C(=O)NC34CC(c5ncc(-c6cnc(Cl)cn6)s5)(C3)C4)o2)CC1]

The title compound was prepared in analogy to the procedure described for the preparation of Example 58, by using 2-chloro-5-iodo-pyrazine instead of 5-chloro-2-iodopyrimidine. The product was purified by preparative HPLC to afford Example 59 as a white solid. MS obsd. (ESI⁺) [(M+H)⁺]: 491.0. ¹H NMR (400 MHz, CDCl₃) δ ppm: 8.69 (d, J=1.2 Hz, 1H), 8.53 (d, J=1.2 Hz, 1H), 8.21 (s, 1H), 7.11 (d, J=3.6 Hz, 1H), 6.89 (s, 1H), 6.64 (d, J=3.6 Hz, 1H), 2.93 (s, 3H), 2.68 (s, 6H), 1.87-1.91 (m, 2H), 1.44-1.48 (m, 2H).

### Example 60

### N-[3-[2-(4-chlorophenyl)triazol-4-yl]-1-bicyclo[1.1.1]pentanyl]-3-(1-methylsulfonylcyclopropyl)-1,2,4-thiadiazole-5-carboxamide

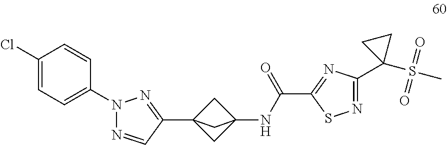 [SMILES: CS(=O)(=O)C1(c2nsc(C(=O)NC34CC(c5cnn(-c6ccc(Cl)cc6)n5)(C3)C4)n2)CC1]

The title compound was prepared in analogy to the procedure described for the preparation of Example 51, by using Int-25 instead of Int-35. The product was purified by preparative HPLC to afford Example 60 as a white solid. MS obsd. (ESI⁺) [(M+H)⁺]: 491.1. ¹H NMR (400 MHz, CDCl₃) δ ppm: 7.95-8.03 (m, 2H), 7.63 (s, 1H), 7.52 (s, 1H), 7.41-7.47 (m, 2H), 3.30 (s, 3H), 2.63 (s, 6H), 2.03 (dt, J=8.8, 4.4 Hz, 2H), 1.79 (q, J=5.0 Hz, 2H).

### Example 61

PHH Natural Infection Assay

Detailed procedures regarding primary human hepatocyte (PHH) HBV natural infection assay are described as below. One tube of frozen PHH (10 million cells) was thawed in 37° C. water bath and then transferred to 20 mL of PHH thawing medium (Sigma, InVitroGRO HT Medium, Cat. 503319) with gently mixing. The cells were then centrifuged at 80 g/min for 5 min, the supernatant was discarded and the tube was refilled with 25 mL of PHH plating medium (Sigma, InVitroGRO CP Medium, Cat. 503317). The tube was shaken very gently to re-suspend all cells, and then 50 μl of cells were transferred to each well of a 384-well collagen I coated plate with appropriate liquid handling equipment, e.g. Integra VIAFLO384 or Agilent Bravo. The cells were then cultured for 24 hours in a cell incubator. For HBV infection, after PHH attachment on the culture plate, the plating medium was removed and replenished with PHH culture medium containing HBV virus. The PHH culture medium was prepared with Dulbecco's Modified Eagle Medium (DMEM)/F12 (1:1 in volume ratio) containing 10% fetal bovine serum (Gibco, Cat.10099141), 5 ng/mL human epidermal growth factor (Gibco, Cat.PHG031 IL), 20 ng/mL dexamethasone (Sigma, Cat.D4902-100 mg), 250 ng/mL human recombinant insulin (Gibco, Cat.41400045) and 100 U/mL penicillin. HBV virus at 200 genome equivalent (GE) per cell with 4% PEG8000 (Sigma, Cat.P1458) containing culture medium were added to the PHH culture medium for infection. The cells were then cultured for 24 hours in cell incubator. Then the cell culture supernatant was removed. The HBV-infected PHH were cultured with sandwich culture method with PHH culture medium containing 1% DMSO and 0.25 mg/mL matrix gel for 72 hours. The supernatant was then refreshed with PHH culture medium containing different concentrations of testing compounds for two times with 72-hour interval. At the end of treatment, the supernatant was collected for viral markers measurements, including HBsAg, HBeAg, HBV DNA and cytotoxicity. HBsAg and HBeAg were detected using alphalisa method using their specific antibodies. For HBV DNA detection, HBV DNA Quantitative Fluorescence Diagnostic Kit (Sansure Biotech Inc.) was used following the manufacture's protocol. Cytotoxicity was determined using Cell Counting Kit-8 (CCK8, Dojindo Molecular Technologies, Inc.).

The compounds of the present invention were tested for their capacity to inhibit HBsAg and HBeAg as described herein. The Examples were tested in the above assay and found to have IC₅₀ below 10 μM. Results of PHH assay are given in Table 3.

TABLE 3
Activity data of compounds of this invention

| | HBsAg IC₅₀ | HBeAg IC₅₀ | TI |
|---|---|---|---|
| Example No. | (μM) | (μM) | (CC₅₀/IC₅₀ HBsAg) |
| Example 1 | 0.16 | 0.12 | 27.88 |
| Example 2 | 0.35 | 0.2 | 20.66 |
| Example 3 | 0.67 | 0.5 | 8.85 |
| Example 4 | 0.17 | 0.12 | 29.65 |
| Example 6 | 0.08 | 0.05 | 81.63 |
| Example 8 | 0.16 | 0.11 | 16.69 |
| Example 10 | 0.28 | 0.2 | 31.11 |
| Example 13 | 0.77 | 0.56 | 12.38 |
| Example 18 | 0.19 | 0.12 | 39.05 |
| Example 19 | 0.21 | 0.16 | >47.62 |
| Example 21 | 0.07 | 0.07 | 29.86 |
| Example 26 | 0.78 | 0.53 | 8.91 |
| Example 27 | 0.13 | 0.09 | 39.46 |
| Example 30 | 0.06 | 0.04 | 148.67 |
| Example 32 | 0.04 | 0.04 | 112.00 |
| Example 33 | 0.44 | 0.37 | >22.73 |
| Example 34 | 0.02 | 0.02 | 181.50 |
| Example 35 | 0.05 | 0.04 | 79.80 |
| Example 37 | 0.47 | 0.37 | 11.94 |
| Example 38 | 0.72 | 0.47 | 8.32 |
| Example 39 | 0.77 | 0.56 | 6.92 |
| Example 40 | 0.14 | 0.1 | 46.00 |
| Example 41 | 0.13 | 0.08 | 65.54 |
| Example 42 | 0.59 | 0.34 | 13.39 |
| Example 43 | 0.09 | 0.08 | >111.11 |
| Example 44 | 0.35 | 0.27 | 12.20 |
| Example 47 | 0.15 | 0.14 | 59.67 |
| Example 48 | 0.07 | 0.07 | 127.43 |
| Example 53 | 0.17 | 0.11 | 21.88 |
| Example 54 | 0.06 | 0.05 | 10.83 |
| Example 55 | 0.09 | 0.07 | 89.00 |
| Example 60 | 0.017 | 0.017 | 261.76 |

### Example 62

### Human Microsome Stability Assay

The human microsomal stability assay is used for early assessment of metabolic stability of a test compound in human liver microsomes.

Human liver microsomes (Cat. NO.: 452117, Corning, USA) were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation time of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 4 below (The detection limit is 6.15 mL/min/kg).

TABLE 4
Human liver microsome stability of
the compounds of present invention

| Example No. | Clearance of human microsome (mL/min/kg) |
|---|---|
| Example 1 | ≤6.15 |
| Example 2 | ≤6.15 |
| Example 3 | ≤6.15 |
| Example 4 | ≤6.15 |
| Example 5 | ≤6.15 |
| Example 6 | ≤6.15 |
| Example 8 | ≤6.15 |
| Example 9 | ≤6.15 |
| Example 10 | ≤6.15 |
| Example 13 | ≤6.15 |
| Example 14 | ≤6.15 |
| Example 16 | ≤6.15 |
| Example 17 | ≤6.15 |
| Example 18 | 8.8 |
| Example 19 | ≤6.15 |
| Example 20 | ≤6.15 |
| Example 21 | 6.66 |
| Example 22 | ≤6.15 |
| Example 23 | ≤6.15 |
| Example 24 | ≤6.15 |
| Example 25 | ≤6.15 |
| Example 26 | ≤6.15 |
| Example 27 | ≤6.15 |
| Example 28 | ≤6.15 |
| Example 29 | ≤6.15 |
| Example 30 | ≤6.15 |
| Example 32 | ≤6.15 |
| Example 33 | ≤6.15 |
| Example 34 | ≤6.15 |
| Example 35 | ≤6.15 |
| Example 36 | 11.99 |
| Example 37 | ≤6.15 |
| Example 38 | ≤6.15 |
| Example 39 | ≤6.15 |
| Example 40 | ≤6.15 |
| Example 41 | 14.14 |
| Example 42 | 8.21 |
| Example 43 | ≤6.15 |
| Example 44 | ≤6.15 |
| Example 47 | ≤6.15 |
| Example 48 | 10.71 |
| Example 56 | ≤6.15 |
| Example 57 | 15.39 |
| Example 59 | 6.89 |
| Example 60 | ≤6.15 |

### Example 63

### Single Dose Pharmacokinetics (PK) Study in Male C57BL/6 Mouse

Pharmacokinetic properties of selected compounds were assessed by single dose PK studies in Male C57BL/6 mouse (vendor: Beijing Vital River Laboratory Animal Technology Co., Ltd). Briefly, two groups of animals were administered a single dose of respective compound intravenously (IV, bolus) at 1 mg/kg or orally (PO, by gavage) at 10 mg/kg or 30 mg/kg. Blood samples (approximately 30 μL) were collected via saphenous vein at 5 min (only for IV), 15 min, 30 min, 1 h, 2 h, 4 h, 7 h, 24 h, 48 h and 72 h (48 h and 72 h only for certain compounds) post-dose. Blood samples were placed into tubes containing EDTA-K2 anticoagulant and centrifuged at 3000 g for 15 min at 4° C. to separate plasma from the samples. After centrifugation, the resulting plasma was transferred to clean tubes for bioanalysis with LC/MS/MS. The pharmacokinetic parameters were calculated using non-compartmental analysis. The volume of distribution (Vss), half-life (T_{1/2}) and clearance (CL) were obtained based on the plasma concentration-time curve after IV dose. The peak concentration (Cₘₐₓ) was recorded directly from experimental observations after PO dose. The area under the plasma concentration-time curve (AUC₀₋ₗₐₛₜ and AUC_{0-24 h}) was calculated using the linear trapezoidal rule up to the last detectable concentration. The bioavailability (F) was calculated based on the dose normalized AUC₀₋ₗₐₛₜ after IV and PO dose.

The Vss of a drug represents the degree to which a drug is distributed in body tissue rather than the plasma. Vss is directly proportional with the amount of drug distributed into tissue. A higher Vss indicates a greater amount of tissue distribution.

Results of PK parameters following IV and PO administration are given in Table 5.

TABLE 5
PK parameters for the compounds of this invention

| | | PO | IV | | | | |
|---|---|---|---|---|---|---|---|
| Example | PO dose | AUC₀₋₂₄ₕ | AUC₀₋₂₄ₕ | CL | V_{SS} | T_{1/2} | F |
| No | (mg/kg) | (h × ng/mL) | (h × ng/mL) | (mL/min/kg) | (L/kg) | (h) | (%) |
| 1 | 30 | 1,310,171 | 42,676 | 0.20 | 0.40 | 23.6 | 102 |
| 30 | 30 | 370,121 | 26,955 | 0.19 | 0.74 | 48.4 | 46 |
| 32 | 30 | 249,161 | 24,074 | 0.43 | 0.64 | 17.8 | 35 |
| 34 | 30 | 269,972 | 24,175* | 0.03 | 0.93 | >40 | 39 |
| 40 | 30 | 80,795 | 14,948 | 0.85 | 0.83 | 12.0 | 18 |
| 41 | 10 | 59,668 | 33,957 | 0.11 | 0.62 | 67.3 | 18 |
| 48 | 10 | 628,857 | 70,178 | 0.07 | 0.29 | 53.7 | 92 |
| 60 | 30 | 154,216 | 38,100 | 0.10 | 0.55 | 62.4 | 14 |

*The IV dose is 0.4 mg/kg; the AUC value is dose-normalized data.

It is to be noted that the term “a” or “an” entity refers to one or more of that entity: for example, “a polypeptide” is understood to represent one or more polypeptides. As such, the terms “a” (or “an”), “one or more,” and “at least one” can be used interchangeably herein.

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A compound of formula (I), 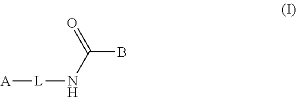
wherein
A is a 5 or 6 membered heteroaryl containing 1, 2, 3, or 4 heteroatom selected from N, O, and S; wherein A is substituted with R¹, or substituted with both R¹ and R², and wherein
R¹ is hydrogen, halogen, C₁₋₆alkyl, haloC₁₋₆alkyl, C₃₋₇cycloalkyl, phenyl, pyridinyl, or pyrimidinyl, wherein each of said phenyl, pyridinyl, and pyrimidinyl is unsubstituted or substituted with one or two substituents independently selected from halogen, C₁₋₆alkyl, and C₁₋₆alkoxy;
R² is hydrogen, halogen, or C₁₋₆alkyl;
L is a C₅₋₁₂cycloalkyl, wherein L is a monocyclic ring or a bicyclic ring, and wherein the bicyclic ring is a bridged, spiro or fused ring;
B is a phenyl, dioxothiolanyl, or a 5 or 6 membered heteroaryl containing 1, 2, 3, or 4 heteroatoms independently selected from N, O, and S; wherein B is substituted with R³, and wherein
R³ is hydrogen, C₁₋₆alkylsulfonylC₃₋₇cycloalkyl-, C₁₋₆alkylsulfonylC₁₋₆alkyl-, or carboxamide;
or a pharmaceutically acceptable salt thereof.

2. A compound according to claim 1, wherein
A is thiazolyl, thiadiazolyl, oxadizolyl, pyrazolyl, trizolyl, tetrazolyl, or pyrimidinyl; wherein A is substituted with R¹, or substituted with both R¹ and R², and wherein
R¹ is hydrogen, halogen, C₁₋₆alkyl, haloC₁₋₆alkyl, C₃₋₇cycloalkyl, phenyl, pyridinyl, or pyrimidinyl, wherein each of said phenyl, pyridinyl, and pyrimidinyl is unsubstituted or substituted with one or two substituents independently selected from halogen, C₁₋₆alkyl, and C₁₋₆alkoxy;
R² is hydrogen, halogen, or C₁₋₆alkyl.

3. A compound according to claim 2, wherein
A is thiazolyl, thiadiazolyl, oxadizolyl, pyrazolyl, trizolyl, tetrazolyl, or pyrimidinyl; wherein A is substituted with R¹, and wherein
R¹ is phenyl or pyridinyl, wherein each of said phenyl and pyridinyl is substituted once or twice with halogen.

4. A compound according to claim 3, wherein
A is 4-(4-chlorophenyl)thiazol-2-yl, 4-(3-chlorophenyl)thiazol-2-yl, 4-(3,4-dichlorophenyl)thiazol-2-yl, 4-(5-chloro-2-pyridyl)thiazol-2-yl, 3-(4-chlorophenyl)-1,2,4-thiadiazol-5-yl, 5-(4-chlorophenyl)thiazol-2-yl, 5-(3,4-dichlorophenyl)thiazol-2-yl, 2-(4-chlorophenyl)thiazol-4-yl, 2-(4-chlorophenyl)triazol-4-yl, 3-(4-chlorophenyl)-1,2,4-oxadiazol-5-yl, 3-(4-fluorophenyl)-1,2,4-oxadiazol-5-yl, 5-(4-chlorophenyl)-1,2,4-oxadiazol-3-yl, 1-(4-chlorophenyl)pyrazol-3-yl, 2-(4-chlorophenyl)tetrazol-5-yl, or 2-(4-chlorophenyl)pyrimidin-4-yl.

5. A compound according to claim 2, wherein
A is thiazolyl, thiadiazolyl, oxadizolyl, pyrazolyl, or trizolyl; wherein A is substituted with R¹, and wherein
R¹ is phenyl or pyridinyl, wherein each of said phenyl and pyridinyl is substituted once with halogen.

6. A compound according to claim 5, wherein
A is 4-(4-chlorophenyl)thiazol-2-yl, 4-(3-chlorophenyl)thiazol-2-yl, 4-(5-chloro-2-pyridyl)thiazol-2-yl, 3-(4-chlorophenyl)-1,2,4-thiadiazol-5-yl, 5-(4-chlorophenyl)thiazol-2-yl, 2-(4-chlorophenyl)triazol-4-yl, 5-(4-chlorophenyl)-1,2,4-oxadiazol-3-yl, or 1-(4-chlorophenyl)pyrazol-3-yl.

7. A compound according to claim 1, wherein
L is 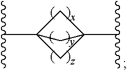
each of x, y, and z is independently an integer of 1, 2, or 3.

8. A compound according to claim 7, wherein
L is

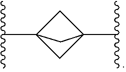 [SMILES: *C12CC(*)(C1)C2]

9. A compound according to claim 1, wherein
B is furanyl, oxazolyl, oxadizolyl, thiadiazolyl, pyrazolyl, pyridinyl, phenyl, pyridinyl, or dioxothiolanyl; wherein B is substituted with R³, and wherein
R³ is hydrogen, C₁₋₆alkylsulfonylC₃₋₇cycloalkyl-, C₁₋₆alkylsulfonylC₁₋₆alkyl-, or carboxamide.

10. A compound according to claim 9, wherein
B is furanyl, oxazolyl, thiadiazolyl, pyrazolyl, or pyridinyl; wherein B is substituted with R³, and wherein
R³ is C₁₋₆alkylsulfonylC₃₋₇cycloalkyl- or C₁₋₆alkylsulfonylC₁₋₆alkyl-.

11. A compound according to claim 10, wherein
B is 5-(1-methylsulfonylcyclopropyl)furan-2-yl, 3-(1-methylsulfonylcyclopropyl)-1,2,4-thiadiazole-5-yl, 2-(1-methylsulfonylcyclopropyl)oxazole-5-yl, 3-(methylsulfonylmethyl)-1,2,4-thiadiazole-5-yl, 3-(1-methyl-1-methylsulfonyl-ethyl)phenyl, 3-(1-methylsulfonylcyclopropyl)pyrazol-1-yl, or 2-(1-methylsulfonylcyclopropyl)oxazol-5-yl.

12. A compound according to claim 9, wherein
B is furanyl or thiadiazolyl; wherein B is substituted with R³, and wherein
R³ is C₁₋₆alkylsulfonylC₃₋₇cycloalkyl-.

13. A compound according to claim 12, wherein
B is 5-(1-methylsulfonylcyclopropyl)furan-2-yl or 3-(1-methylsulfonylcyclopropyl)-1,2,4-thiadiazole-5-yl.

14. A compound according to claim 1, wherein
A is thiazolyl, thiadiazolyl, oxadizolyl, pyrazolyl, trizolyl, tetrazolyl, or pyrimidinyl; wherein A is substituted with R¹, and wherein
R¹ is phenyl or pyridinyl, wherein each of said phenyl and pyridinyl is substituted once or twice with halogen;
L is 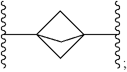 [SMILES: *C12CC(*)(C1)C2]
and
B is furanyl, oxazolyl, thiadiazolyl, pyrazolyl, or pyridinyl; wherein B is substituted with R³, and wherein
R³ is C₁₋₆alkylsulfonylC₃₋₇cycloalkyl- or C₁₋₆alkylsulfonylC₁₋₆alkyl-.

15. A compound according to claim 14, wherein
A is 4-(4-chlorophenyl)thiazol-2-yl, 4-(3-chlorophenyl)thiazol-2-yl, 4-(3,4-dichlorophenyl)thiazol-2-yl, 4-(5-chloro-2-pyridyl)thiazol-2-yl, 3-(4-chlorophenyl)-1,2,4-thiadiazol-5-yl, 5-(4-chlorophenyl)thiazol-2-yl, 5-(3,4-dichlorophenyl)thiazol-2-yl, 2-(4-chlorophenyl)thiazol-4-yl, 2-(4-chlorophenyl)triazol-4-yl, 3-(4-chlorophenyl)-1,2,4-oxadiazol-5-yl, 3-(4-fluorophenyl)-1,2,4-oxadiazol-5-yl, 5-(4-chlorophenyl)-1,2,4-oxadiazol-3-yl, 1-(4-chlorophenyl)pyrazol-3-yl, 2-(4-chlorophenyl)tetrazol-5-yl, or 2-(4-chlorophenyl)pyrimidin-4-yl;
L is 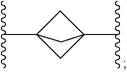 [SMILES: *C12CC(*)(C1)C2]
and
B is 5-(1-methylsulfonylcyclopropyl)furan-2-yl, 3-(1-methylsulfonylcyclopropyl)-1,2,4-thiadiazole-5-yl, 2-(1-methylsulfonylcyclopropyl)oxazole-5-yl, 3-(methylsulfonylmethyl)-1,2,4-thiadiazole-5-yl, 3-(1-methyl-1-methylsulfonyl-ethyl)phenyl, 3-(1-methylsulfonylcyclopropyl)pyrazol-1-yl, or 2-(1-methylsulfonylcyclopropyl)oxazol-5-yl.

16. A compound according to claim 1, wherein
A is thiazolyl, thiadiazolyl, oxadizolyl, pyrazolyl, or trizolyl; wherein A is substituted with R¹, and wherein
R¹ is phenyl or pyridinyl, wherein each of said phenyl and pyridinyl is substituted once with halogen;
L is 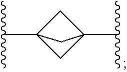 [SMILES: *C12CC(*)(C1)C2]
and
B is furanyl or thiadiazolyl; wherein B is substituted with R³, and wherein
R³ is C₁₋₆alkylsulfonylC₃₋₇cycloalkyl-.

17. A compound according to claim 16, wherein
A is 4-(4-chlorophenyl)thiazol-2-yl, 4-(3-chlorophenyl)thiazol-2-yl, 4-(5-chloro-2-pyridyl)thiazol-2-yl, 3-(4-chlorophenyl)-1,2,4-thiadiazol-5-yl, 5-(4-chlorophenyl)thiazol-2-yl, 2-(4-chlorophenyl)triazol-4-yl, 5-(4-chlorophenyl)-1,2,4-oxadiazol-3-yl, or 1-(4-chlorophenyl)pyrazol-3-yl;
L is 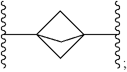 [SMILES: *C12CC(*)(C1)C2]
and
B is 5-(1-methylsulfonylcyclopropyl)furan-2-yl or 3-(1-methylsulfonylcyclopropyl)-1,2,4-thiadiazole-5-yl.

18. A compound selected from:
N-[3-[4-(4-chlorophenyl)thiazol-2-yl]-1-bicyclo[1.1.1]pentanyl]-5-(1-methylsulfonylcyclopropyl)furan-2-carboxamide;
N-[3-[4-(3-chlorophenyl)thiazol-2-yl]-1-bicyclo[1.1.1]pentanyl]-5-(1-methylsulfonylcyclopropyl)furan-2-carboxamide;
N-[3-[4-(2-chlorophenyl)thiazol-2-yl]-1-bicyclo[1.1.1]pentanyl]-5-(1-methylsulfonylcyclopropyl)furan-2-carboxamide;
N-[3-[4-(3-chlorophenyl)thiazol-2-yl]-1-bicyclo[1.1.1]pentanyl]-3-(1-methylsulfonylcyclopropyl)-1,2,4-thiadiazole-5-carboxamide;
N-[3-[4-(4-chlorophenyl)thiazol-2-yl]-1-bicyclo[1.1.1]pentanyl]-2-(methylsulfonylmethyl)oxazole-5-carboxamide;
N-[3-[4-(4-chlorophenyl)thiazol-2-yl]-1-bicyclo[1.1.1]pentanyl]-2-(1-methylsulfonylcyclopropyl)oxazole-5-carboxamide;
N-[3-[4-(4-chlorophenyl)thiazol-2-yl]-1-bicyclo[1.1.1]pentanyl]-5-(1-methylsulfonylcyclopropyl)-1,3,4-oxadiazole-2-carboxamide;
N-[3-[4-(3,4-dichlorophenyl)thiazol-2-yl]-1-bicyclo[1.1.1]pentanyl]-5-(1-methylsulfonylcyclopropyl)furan-2-carboxamide;
N-[3-[4-(6-chloro-3-pyridyl)thiazol-2-yl]-1-bicyclo[1.1.1]pentanyl]-5-(1-methylsulfonylcyclopropyl)furan-2-carboxamide;
N-[3-[4-(5-chloro-2-pyridyl)thiazol-2-yl]-1-bicyclo[1.1.1]pentanyl]-5-(1-methylsulfonylcyclopropyl)furan-2-carboxamide;
5-(1-methylsulfonylcyclopropyl)-N-[3-[4-(2-pyridyl)thiazol-2-yl]-1-bicyclo[1.1.1]pentanyl]furan-2-carboxamide;
5-(1-methylsulfonylcyclopropyl)-N-[3-(4-pyrimidin-5-ylthiazol-2-yl)-1-bicyclo[1.1.1]pentanyl]furan-2-carboxamide;
5-(1-methylsulfonylcyclopropyl)-N-[3-(4-phenylthiazol-2-yl)-1-bicyclo[1.1.1]pentanyl]furan-2-carboxamide;
N-[3-(4-cyclopropylthiazol-2-yl)-1-bicyclo[1.1.1]pentanyl]-5-(1-methylsulfonylcyclopropyl)furan-2-carboxamide;
N-[3-(4-bromothiazol-2-yl)-1-bicyclo[1.1.1]pentanyl]-5-(1-methylsulfonylcyclopropyl)furan-2-carboxamide;
5-(1-methylsulfonylcyclopropyl)-N-[3-[4-(trifluoromethyl)thiazol-2-yl]-1-bicyclo[1.1.1]pentanyl]furan-2-carboxamide;
5-(1-methylsulfonylcyclopropyl)-N-[3-(3-phenyl-1,2,4-thiadiazol-5-yl)-1-bicyclo[1.1.1]pentanyl]furan-2-carboxamide;
N-[3-[3-(4-chlorophenyl)-1,2,4-thiadiazol-5-yl]-1-bicyclo[1.1.1]pentanyl]-5-(1-methylsulfonylcyclopropyl)furan-2-carboxamide;
N-[3-[5-(4-chlorophenyl)thiazol-2-yl]-1-bicyclo[1.1.1]pentanyl]-5-(1-methylsulfonylcyclopropyl)furan-2-carboxamide;
N-[3-[5-(4-chlorophenyl)thiazol-2-yl]-1-bicyclo[1.1.1]pentanyl]-5-(1-methylsulfonylcyclopropyl)-1,3,4-oxadiazole-2-carboxamide;
N-[3-[5-(3,4-dichlorophenyl)thiazol-2-yl]-1-bicyclo[1.1.1]pentanyl]-5-(1-methylsulfonylcyclopropyl)furan-2-carboxamide;
5-(1-methylsulfonylcyclopropyl)-N-[3-(5-phenylthiazol-2-yl)-1-bicyclo[1.1.1]pentanyl]furan-2-carboxamide;
N-[3-(5-cyclopropylthiazol-2-yl)-1-bicyclo[1.1.1]pentanyl]-5-(1-methylsulfonylcyclopropyl)furan-2-carboxamide;
N-[3-(5-bromothiazol-2-yl)-1-bicyclo[1.1.1]pentanyl]-5-(1-methylsulfonylcyclopropyl)furan-2-carboxamide;
5-(1-methylsulfonylcyclopropyl)-N-[3-(5-phenyl-1,2,4-thiadiazol-3-yl)-1-bicyclo[1.1.1]pentanyl]furan-2-carboxamide;
5-(1-methylsulfonylcyclopropyl)-N-[3-(2-phenylthiazol-4-yl)-1-bicyclo[1.1.1]pentanyl]furan-2-carboxamide;
N-[3-[2-(4-chlorophenyl)thiazol-4-yl]-1-bicyclo[1.1.1]pentanyl]-5-(1-methylsulfonylcyclopropyl)furan-2-carboxamide;
N-[3-[5-(4-chlorophenyl)-1,3,4-oxadiazol-2-yl]-1-bicyclo[1.1.1]pentanyl]-5-(1-methylsulfonylcyclopropyl)furan-2-carboxamide;
N-[3-[1-(4-chlorophenyl)triazol-4-yl]-1-bicyclo[1.1.1]pentanyl]-5-(1-methylsulfonylcyclopropyl)furan-2-carboxamide;
N-[3-[2-(4-chlorophenyl)triazol-4-yl]-1-bicyclo[1.1.1]pentanyl]-5-(1-methylsulfonylcyclopropyl)furan-2-carboxamide;
N-[3-[2-(4-chlorophenyl)triazol-4-yl]-1-bicyclo[1.1.1]pentanyl]-1,1-dioxo-thiolane-3-carboxamide;
N-[3-[3-(4-chlorophenyl)-1,2,4-oxadiazol-5-yl]-1-bicyclo[1.1.1]pentanyl]-5-(1-methylsulfonylcyclopropyl)furan-2-carboxamide;
N-[3-[3-(4-chlorophenyl)-1,2,4-oxadiazol-5-yl]-1-bicyclo[1.1.1]pentanyl]-3-(methylsulfonylmethyl)-1,2,4-thiadiazole-5-carboxamide;
N-[3-[3-(4-chlorophenyl)-1,2,4-oxadiazol-5-yl]-1-bicyclo[1.1.1]pentanyl]-3-(1-methylsulfonylcyclopropyl)-1,2,4-thiadiazole-5-carboxamide;
N-[3-[3-(4-fluorophenyl)-1,2,4-oxadiazol-5-yl]-1-bicyclo[1.1.1]pentanyl]-3-(1-methylsulfonylcyclopropyl)-1,2,4-thiadiazole-5-carboxamide;
N4-[3-[3-(4-chlorophenyl)-1,2,4-oxadiazol-5-yl]-1-bicyclo[1.1.1]pentanyl]pyridine-2,4-dicarboxamide;
N-[3-[3-(4-chlorophenyl)-1,2,4-oxadiazol-5-yl]-1-bicyclo[1.1.1]pentanyl]-3-(1-methyl-1-methylsulfonyl-ethyl)benzamide;
N-[3-[3-(4-chlorophenyl)-1,2,4-oxadiazol-5-yl]-1-bicyclo[1.1.1]pentanyl]-6-(1-methyl-1-methylsulfonyl-propyl)pyridine-2-carboxamide;
N-[3-[3-(4-chlorophenyl)-1,2,4-oxadiazol-5-yl]-1-bicyclo[1.1.1]pentanyl]-2-(1-methyl-1-methylsulfonyl-propyl)pyridine-4-carboxamide;
N-[3-[5-(4-chlorophenyl)-1,2,4-oxadiazol-3-yl]-1-bicyclo[1.1.1]pentanyl]-5-(1-methylsulfonylcyclopropyl)furan-2-carboxamide;
N-[3-[5-(4-chlorophenyl)-1,2,4-oxadiazol-3-yl]-1-bicyclo[1.1.1]pentanyl]-3-(1-methylsulfonylcyclopropyl)-1,2,4-thiadiazole-5-carboxamide;
N-[3-[1-(4-chlorophenyl)pyrazol-3-yl]-1-bicyclo[1.1.1]pentanyl]-5-(1-methylsulfonylcyclopropyl)furan-2-carboxamide;
N-[3-[1-(4-chlorophenyl)pyrazol-3-yl]-1-bicyclo[1.1.1]pentanyl]-3-(1-methylsulfonylcyclopropyl)-1,2,4-thiadiazole-5-carboxamide;
N-[3-[2-(4-chlorophenyl)tetrazol-5-yl]-1-bicyclo[1.1.1]pentanyl]-5-(1-methylsulfonylcyclopropyl)furan-2-carboxamide;
N-[3-[4-(4-chlorophenyl)pyrimidin-2-yl]-1-bicyclo[1.1.1]pentanyl]-5-(1-methylsulfonylcyclopropyl)furan-2-carboxamide;
N-[3-[4-(4-chlorophenyl)pyrimidin-2-yl]-1-bicyclo[1.1.1]pentanyl]-3-(1-methylsulfonylcyclopropyl)-1,2,4-thiadiazole-5-carboxamide;
N-[3-[2-(4-chlorophenyl)pyrimidin-4-yl]-1-bicyclo[1.1.1]pentanyl]-5-(1-methylsulfonylcyclopropyl)furan-2-carboxamide;
N-[3-[2-(4-chlorophenyl)pyrimidin-4-yl]-1-bicyclo[1.1.1]pentanyl]-3-(1-methylsulfonylcyclopropyl)-1,2,4-thiadiazole-5-carboxamide;
N-[3-[5-bromo-2-(4-chlorophenyl)triazol-4-yl]-1-bicyclo[1.1.1]pentanyl]-5-(1-methylsulfonylcyclopropyl)furan-2-carboxamide;
N-[3-[2-(4-chlorophenyl)-5-methyl-triazol-4-yl]-1-bicyclo[1.1.1]pentanyl]-5-(1-methylsulfonylcyclopropyl)furan-2-carboxamide;
3-(1-methylsulfonylcyclopropyl)-N-[3-[2-(p-tolyl)triazol-4-yl]-1-bicyclo[1.1.1]pentanyl]-1,2,4-thiadiazole-5-carboxamide;
N-[3-[2-(4-methoxyphenyl)triazol-4-yl]-1-bicyclo[1.1.1]pentanyl]-3-(1-methylsulfonylcyclopropyl)-1,2,4-thiadiazole-5-carboxamide;
N-[3-[4-(4-chlorophenyl)thiazol-2-yl]-1-bicyclo[1.1.1]pentanyl]-3-(1-methylsulfonylcyclopropyl)pyrazole-1-carboxamide;
N-[3-[5-(4-chlorophenyl)thiazol-2-yl]-1-bicyclo[1.1.1]pentanyl]-3-(1-methylsulfonylcyclopropyl)pyrazole-1-carboxamide;
N-[3-[5-(4-chlorophenyl)thiazol-2-yl]-1-bicyclo[1.1.1]pentanyl]-2-(1-methylsulfonylcyclopropyl)oxazole-5-carboxamide;
N-[3-[5-(6-chloro-3-pyridyl)thiazol-2-yl]-1-bicyclo[1.1.1]pentanyl]-5-(1-methylsulfonylcyclopropyl)furan-2-carboxamide;
N-[3-[5-(5-chloro-2-pyridyl)thiazol-2-yl]-1-bicyclo[1.1.1]pentanyl]-5-(1-methylsulfonylcyclopropyl)furan-2-carboxamide;
N-[3-[5-(5-chloropyrimidin-2-yl)thiazol-2-yl]-1-bicyclo[1.1.1]pentanyl]-5-(1-methylsulfonylcyclopropyl)furan-2-carboxamide;
N-[3-[5-(5-chloropyrazin-2-yl)thiazol-2-yl]-1-bicyclo[1.1.1]pentanyl]-5-(1-methylsulfonylcyclopropyl)furan-2-carboxamide; or,
N-[3-[2-(4-chlorophenyl)triazol-4-yl]-1-bicyclo[1.1.1]pentanyl]-3-(1-methylsulfonylcyclopropyl)-1,2,4-thiadiazole-5-carboxamide;
or a pharmaceutically acceptable salt thereof.

19. A process for the preparation of a compound according to claim 1 comprising the following step: 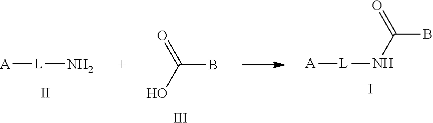
coupling of amine II and acid III with a coupling reagent and a base, in a solvent;
wherein the coupling reagent is selected from O-(7-azabenzotriazol-1-yl)-N,N,N′,N′-tetramethyluronium hexafluorophosphate, N-ethyl-N′-(3-dimethylaminopropyl)carbodiimide hydrochloride, or 1-ethyl-3-(3-dimethylaminopropyl)carbodiimide, the base is selected from selected from dichloromethane, N,N-dimethylformamide, dimethyl sulfoxide, or 1-methyl-pyrrolidin-2-one.

20. A compound or a pharmaceutically acceptable salt thereof according to claim 1, when manufactured according to the process of claim 19.

21. A pharmaceutical composition comprising a compound or a pharmaceutically acceptable salt thereof according to claim 1, and a pharmaceutically acceptable excipient.

22. A compound or a pharmaceutically acceptable salt thereof according to claim 1 for use as therapeutically active substance.

23. A compound or a pharmaceutically acceptable salt thereof according to claim 1 for use in the treatment or prophylaxis of HBV infection.

24. The use of a compound or a pharmaceutically acceptable salt thereof according to claim 1 for the treatment or prophylaxis of HBV infection.

25. The use of a compound or a pharmaceutically acceptable salt thereof according to claim 1 for the inhibition of HBsAg.

26. The use of a compound or a pharmaceutically acceptable salt thereof according to claim 1 for the inhibition of HBeAg.

27. The use of a compound or a pharmaceutically acceptable salt thereof according to claim 1 for the preparation of a medicament for the treatment or prophylaxis of HBV infection.

28. A method for the treatment or prophylaxis of HBV infection, which method comprises administering an effective amount of a compound claim 1, or, a pharmaceutically acceptable salt thereof.
